SEQUENCE LISTING (49 sequences)

SEQ ID NO: 1 - DNA, 3822 bp, SARS-CoV-2 virus
  source 1..3822: mol_type = genomic DNA
atgtttgtttttcttgttttattgccactagtctctagtcagtgtgttaatcttacaaccagaactcaattaccccctgcatacactaattctttcacacgtggtgtttattaccctgacaaagttttcagatcctcagttttacattcaactcaggacttgttcttacctttcttttccaatgttacttggttccatgctatacatgtctctgggaccaatggtactaagaggtttgataaccctgtcctaccatttaatgatggtgtttattttgcttccactgagaagtctaacataataagaggctggatttttggtactactttagattcgaagacccagtccctacttattgttaataacgctactaatgttgttattaaagtctgtgaatttcaattttgtaatgatccatttttgggtgtttattaccacaaaaacaacaaaagttggatggaaagtgagttcagagtttattctagtgcgaataattgcacttttgaatatgtctctcagccttttcttatggaccttgaaggaaaacagggtaatttcaaaaatcttagggaatttgtgtttaagaatattgatggttattttaaaatatattctaagcacacgcctattaatttagtgcgtgatctccctcagggtttttcggctttagaaccattggtagatttgccaataggtattaacatcactaggtttcaaactttacttgctttacatagaagttatttgactcctggtgattcttcttcaggttggacagctggtgctgcagcttattatgtgggttatcttcaacctaggacttttctattaaaatataatgaaaatggaaccattacagatgctgtagactgtgcacttgaccctctctcagaaacaaagtgtacgttgaaatccttcactgtagaaaaaggaatctatcaaacttctaactttagagtccaaccaacagaatctattgttagatttcctaatattacaaacttgtgcccttttggtgaagtttttaacgccaccagatttgcatctgtttatgcttggaacaggaagagaatcagcaactgtgttgctgattattctgtcctatataattccgcatcattttccacttttaagtgttatggagtgtctcctactaaattaaatgatctctgctttactaatgtctatgcagattcatttgtaattagaggtgatgaagtcagacaaatcgctccagggcaaactggaaagattgctgattataattataaattaccagatgattttacaggctgcgttatagcttggaattctaacaatcttgattctaaggttggtggtaattataattacctgtatagattgtttaggaagtctaatctcaaaccttttgagagagatatttcaactgaaatctatcaggccggtagcacaccttgtaatggtgttgaaggttttaattgttactttcctttacaatcatatggtttccaacccactaatggtgttggttaccaaccatacagagtagtagtactttcttttgaacttctacatgcaccagcaactgtttgtggacctaaaaagtctactaatttggttaaaaacaaatgtgtcaatttcaacttcaatggtttaacaggcacaggtgttcttactgagtctaacaaaaagtttctgcctttccaacaatttggcagagacattgctgacactactgatgctgtccgtgatccacagacacttgagattcttgacattacaccatgttcttttggtggtgtcagtgttataacaccaggaacaaatacttctaaccaggttgctgttctttatcaggatgttaactgcacagaagtccctgttgctattcatgcagatcaacttactcctacttggcgtgtttattctacaggttctaatgtttttcaaacacgtgcaggctgtttaataggggctgaacatgtcaacaactcatatgagtgtgacatacccattggtgcaggtatatgcgctagttatcagactcagactaattctcctcggcgggcacgtagtgtagctagtcaatccatcattgcctacactatgtcacttggtgcagaaaattcagttgcttactctaataactctattgccatacccacaaattttactattagtgttaccacagaaattctaccagtgtctatgaccaagacatcagtagattgtacaatgtacatttgtggtgattcaactgaatgcagcaatcttttgttgcaatatggcagtttttgtacacaattaaaccgtgctttaactggaatagctgttgaacaagacaaaaacacccaagaagtttttgcacaagtcaaacaaatttacaaaacaccaccaattaaagattttggtggttttaatttttcacaaatattaccagatccatcaaaaccaagcaagaggtcatttattgaagatctacttttcaacaaagtgacacttgcagatgctggcttcatcaaacaatatggtgattgccttggtgatattgctgctagagacctcatttgtgcacaaaagtttaacggccttactgttttgccacctttgctcacagatgaaatgattgctcaatacacttctgcactgttagcgggtacaatcacttctggttggacctttggtgcaggtgctgcattacaaataccatttgctatgcaaatggcttataggtttaatggtattggagttacacagaatgttctctatgagaaccaaaaattgattgccaaccaatttaatagtgctattggcaaaattcaagactcactttcttccacagcaagtgcacttggaaaacttcaagatgtggtcaaccaaaatgcacaagctttaaacacgcttgttaaacaacttagctccaattttggtgcaatttcaagtgttttaaatgatatcctttcacgtcttgacaaagttgaggctgaagtgcaaattgataggttgatcacaggcagacttcaaagtttgcagacatatgtgactcaacaattaattagagctgcagaaatcagagcttctgctaatcttgctgctactaaaatgtcagagtgtgtacttggacaatcaaaaagagttgatttttgtggaaagggctatcatcttatgtccttccctcagtcagcacctcatggtgtagtcttcttgcatgtgacttatgtccctgcacaagaaaagaacttcacaactgctcctgccatttgtcatgatggaaaagcacactttcctcgtgaaggtgtctttgtttcaaatggcacacactggtttgtaacacaaaggaatttttatgaaccacaaatcattactacagacaacacatttgtgtctggtaactgtgatgttgtaataggaattgtcaacaacacagtttatgatcctttgcaacctgaattagactcattcaaggaggagttagataaatattttaagaatcatacatcaccagatgttgatttaggtgacatctctggcattaatgcttcagttgtaaacattcaaaaagaaattgaccgcctcaatgaggttgccaagaatttaaatgaatctctcatcgatctccaagaacttggaaagtatgagcagtatataaaatggccatggtacatttggctaggttttatagctggcttgattgccatagtaatggtgacaattatgctttgctgtatgaccagttgctgtagttgtctcaagggctgttgttcttgtggatcctgctgcaaatttgatgaagacgactctgagccagtgctcaaaggagtcaaattacattacacataa

SEQ ID NO: 2 - protein, 1260 aa, SARS-CoV-2 virus
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 3 - DNA, 3786 bp, synthetic construct
  misc_feature 1..3786: note = FL-S (Wuhan) coding sequence
  source 1..3786: mol_type = other DNA
cagtgcgtgaacctgaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcaggacgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagtacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctactaa

SEQ ID NO: 4 - protein, 1260 aa, synthetic construct
  REGION 1..1260: note = FL-S (Wuhan)
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 5 - protein, 1260 aa, synthetic construct
  REGION 1..1260: note = FL-S (Wuhan), without stabilzing prolines
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 6 - DNA, 3777 bp, synthetic construct
  misc_feature 1..3777: note = FL-S (Beta) coding sequence
  source 1..3777: mol_type = other DNA
cagtgcgtgaacttcaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgccaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcggcctgccacagggcttcagcgccctggagccactggtggatctgcccatcggcatcaacatcaccaggtttcagacactgcatatctcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaatatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtgaagggctttaactgttatttccctctgcagtcttacggcttccagccaacctacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcagggcgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgtggagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagtacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctactaa

SEQ ID NO: 7 - protein, 1257 aa, synthetic construct
  REGION 1..1257: note = FL-S (Beta)
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 8 - protein, 1257 aa, synthetic construct
  REGION 1..1257: note = FL-S (Beta), without stabilizing prolines
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 9 - DNA, 3780 bp, synthetic construct
  misc_feature 1..3780: note = FL-S (Delta) coding sequence
  source 1..3780: mol_type = other DNA
cagtgcgtgaacctgaggacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctggacgtgtactatcataagaacaataagagctggatggagtctggcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatcggtacaggctgtttcggaagagcaatctgaagccattcgagagagacatctccacagagatctaccaggctggcagcaagccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcagggcgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccagaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcagaatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagtacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctactaa

SEQ ID NO: 10 - protein, 1258 aa, synthetic construct
  REGION 1..1258: note = FL-S (Delta)
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 11 - protein, 1258 aa, synthetic construct
  REGION 1..1258: note = FL-S (Delta), without stabilizing prolines
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - DNA, 3777 bp, synthetic construct
  misc_feature 1..3777: note = FL-S (Omicron) coding sequence
  source 1..3777: mol_type = other DNA
caatgcgtgaatctgaccacaagaacacagctgcctcccgcctataccaactccttcaccagaggagtgtactaccccgacaaggtgttcagatctagcgtgctgcactccacacaggacctgttcctgcctttcttctccaacgtcacatggttccacgtgatctccggaacaaacggcaccaagagattcgacaaccctgtgctgccgttcaacgacggcgtgtactttgcttcaatcgagaagtccaacataatcagaggctggatcttcgggacaaccctggacagcaaaacccagtccctgctgatcgtgaacaacgccaccaatgtggtgatcaaggtgtgcgagttccagttctgcaacgatcccttcctggaccacaagaacaacaagtcctggatggaatccgagtttagagtgtactcctccgccaataactgtacattcgagtacgtgtcccagcctttcctgatggacctggaaggtaaacagggcaacttcaaaaacctgagagagttcgtgtttaagaacatcgacggctactttaagatctactcgaagcacacacctataatcgtgcgcgagcccgaggacctgcctcaaggcttcagcgccctggagcctctggtggacctgccaatcggaatcaacatcacccgctttcagaccctcctggctctgcacagatcctacctgactcctggcgattcttcttccggctggaccgctggtgccgccgcttactacgtgggctacctgcagcctcggaccttcctgctgaagtacaacgagaacggcaccatcaccgacgccgtggactgcgctcttgaccccctgtctgagactaagtgcaccctgaagtccttcaccgtggaaaagggcatctaccagacctccaacttccgggtgcagcccaccgagtctatcgtgcggttccccaacatcaccaatctgtgtcctttcgacgaggtgttcaatgctacccggtttgcctctgtgtacgcttggaacagaaagagaatctcaaactgcgtggccgactactccgttctgtacaacctggccccattcttcacctttaagtgctacggcgtctctcctaccaagctcaacgacctgtgcttcacaaacgtgtacgccgactctttcgtgattagaggcgacgaagtgagacagatcgctccgggccagaccggaaacatcgccgactacaactacaagctgccagacgacttcaccggctgtgtgatcgcctggaactccaacaagctggactccaaggtgtccggcaattacaactacctgtaccggctgtttcggaagtccaacctgaagcccttcgagcgggatatttccaccgagatctaccaggccggcaacaagccttgtaacggcgttgctggcttcaactgctactttcctctgaagtcctatagcttcaggcctacttacggagtgggccaccagccttacagagtggtcgtgctgtcttttgagctgctgcatgcccctgctacagtctgcggacctaagaaaagcaccaatctggtgaagaacaagtgcgtgaacttcaacttcaatggcctgaaaggcacaggcgtgctgaccgagtctaacaagaagttcctgcccttccagcagttcggtcgggacatagctgacaccaccgatgccgtgcgggaccctcagaccctggaaatcctggatatcaccccttgctccttcgggggagtttccgtgatcacccctggcaccaacacctcaaaccaggtggctgtgctgtatcaaggcgttaactgtaccgaagtgcccgtggccattcacgctgaccagctgaccccaacctggcgggtttactccaccggctcgaacgtgttccagaccagagccggctgcctgatcggcgccgagtacgtcaacaattcttacgagtgtgacatccccatcggcgccggaatctgtgcctcctaccagacacagaccaagtctcatggcggggcctcttccgtggctagccagtccatcatcgcctacaccatgtccctgggagccgaaaactctgtggcctactctaacaactccatcgctatccctactaactttaccatctccgtgaccacagagatcctgcctgtctccatgaccaagacctctgtggactgcactatgtacatctgcggcgactccaccgagtgctctaatctgctgctgcaatatggctccttctgcacccagctgaaacgggctctgaccggcatcgccgtggaacaggacaagaacacacaggaagtgttcgcccaggtgaagcagatctacaagacccctccaatcaaatacttcggcgggttcaacttctcccagattctgcctgatccttctaagcctagcaagcggtcctttatcgaggatctgctgttcaacaaagttacactcgccgatgccggcttcatcaagcagtacggcgactgcctcggagatatcgccgccagagacctgatctgcgctcagaagttcaagggcctgacggtgctgcctcctctcctgaccgatgagatgatcgcccagtacacctctgccctgctggctggcaccatcaccagcggatggaccttcggcgctggcgctgccctacagatccctttcgccatgcagatggcctaccggtttaacggcatcggcgtgacccagaacgtgctgtacgagaaccagaagctgatcgcgaaccagttcaactccgcgatcggcaagatccaggactctctgtccagtaccgcaagtgcactcggcaagctgcaggatgtggtgaaccacaacgcccaggctctgaacaccctcgtgaagcaactgtctagcaagttcggcgccatctcttctgttctgaacgatatcttctcgcggctcgacccacctgaggccgaggtgcaaatcgaccggctgatcaccggcagactgcagtccctgcaaacttacgtgacccagcagctaatcagagctgccgaaatccgtgcatctgccaacctggctgctaccaagatgtcggaatgcgtgctgggccagtctaagagagtggacttctgtggcaagggctaccacctgatgtccttccctcaatctgctcctcacggcgttgtctttctgcatgtgacctatgtgcctgcccaggagaagaacttcacgacagctcctgctatctgtcacgacggcaaggcccactttcctagagaaggcgtgttcgtgtctaatggaacccactggttcgtgacccaaagaaacttctacgaaccccagatcatcacaaccgacaacaccttcgtctccggcaactgcgacgtggtgatcggcatcgtgaataacaccgtgtacgaccctctccagcctgaactggatagcttcaaagaggaactggataagtacttcaagaaccacacttctcctgatgtggacctgggcgacatctccggcatcaacgccagcgtggtgaacatccagaaagaaatcgacagactgaacgaggtggccaagaacctgaacgagtctctgatcgacctgcaggagctgggcaagtacgagcagtacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctactaa

SEQ ID NO: 13 - protein, 1257 aa, synthetic construct
  REGION 1..1257: note = FL-S (Omicron)
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGGASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 1257 aa, synthetic construct
  REGION 1..1257: note = FL-S (Omicron), without stabilizing prolines
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGGASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - DNA, 3855 bp, synthetic construct
  misc_feature 1..3855: note = FL-S-HA (Wuhan) coding sequence
  source 1..3855: mol_type = other DNA
atgttcgtgtttctggtgctgctgccactggtgtccagccagtgcgtgaacctgaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcaggacgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagggtacctacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctacccatacgatgttccagattacgcttaa

SEQ ID NO: 16 - protein, 1271 aa, synthetic construct
  REGION 1..1271: note = FL-S-HA (Wuhan)
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTYIKWPWYIWLGFIAGLIAIVMVTIMLCCMTSCCSCLKGCCSCGSCCKFDEDDSEPVLKGVKLHYTYPYDVPDYA

SEQ ID NO: 17 - DNA, 3846 bp, synthetic construct
  misc_feature 1..3846: note = FL-S-HA (Beta) coding sequence
  source 1..3846: mol_type = other DNA
atgttcgtgtttctggtgctgctgccactggtgtccagccagtgcgtgaacttcaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgccaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcggcctgccacagggcttcagcgccctggagccactggtggatctgcccatcggcatcaacatcaccaggtttcagacactgcatatctcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaatatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtgaagggctttaactgttatttccctctgcagtcttacggcttccagccaacctacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcagggcgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgtggagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagggtacctacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctacccatacgatgttccagattacgcttaa

SEQ ID NO: 18 - protein, 1268 aa, synthetic construct
  REGION 1..1268: note = FL-S-HA (Beta)
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTYIKWPWYIWLGFIAGLIAIVMVTIMLCCMTSCCSCLKGCCSCGSCCKFDEDDSEPVLKGVKLHYTYPYDVPDYA

SEQ ID NO: 19 - DNA, 3849 bp, synthetic construct
  misc_feature 1..3849: note = FL-S-HA (Delta) coding sequence
  source 1..3849: mol_type = other DNA
atgttcgtgtttctggtgctgctgccactggtgtccagccagtgcgtgaacctgaggacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctggacgtgtactatcataagaacaataagagctggatggagtctggcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatcggtacaggctgtttcggaagagcaatctgaagccattcgagagagacatctccacagagatctaccaggctggcagcaagccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcagggcgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccagaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcagaatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagggtacctacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctacccatacgatgttccagattacgcttaa

SEQ ID NO: 20 - protein, 1269 aa, synthetic construct
  REGION 1..1269: note = FL-S-HA (Delta)
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTYIKWPWYIWLGFIAGLIAIVMVTIMLCCMTSCCSCLKGCCSCGSCCKFDEDDSEPVLKGVKLHYTYPYDVPDYA

SEQ ID NO: 21 - DNA, 3846 bp, synthetic construct
  misc_feature 1..3846: note = FL-S-HA (Omicron)
  source 1..3846: mol_type = other DNA
atgtttgtgtttctagtgctactaccactggtgagctctcaatgcgtgaatctgaccacaagaacacagctgcctcccgcctataccaactccttcaccagaggagtgtactaccccgacaaggtgttcagatctagcgtgctgcactccacacaggacctgttcctgcctttcttctccaacgtcacatggttccacgtgatctccggaacaaacggcaccaagagattcgacaaccctgtgctgccgttcaacgacggcgtgtactttgcttcaatcgagaagtccaacataatcagaggctggatcttcgggacaaccctggacagcaaaacccagtccctgctgatcgtgaacaacgccaccaatgtggtgatcaaggtgtgcgagttccagttctgcaacgatcccttcctggaccacaagaacaacaagtcctggatggaatccgagtttagagtgtactcctccgccaataactgtacattcgagtacgtgtcccagcctttcctgatggacctggaaggtaaacagggcaacttcaaaaacctgagagagttcgtgtttaagaacatcgacggctactttaagatctactcgaagcacacacctataatcgtgcgcgagcccgaggacctgcctcaaggcttcagcgccctggagcctctggtggacctgccaatcggaatcaacatcacccgctttcagaccctcctggctctgcacagatcctacctgactcctggcgattcttcttccggctggaccgctggtgccgccgcttactacgtgggctacctgcagcctcggaccttcctgctgaagtacaacgagaacggcaccatcaccgacgccgtggactgcgctcttgaccccctgtctgagactaagtgcaccctgaagtccttcaccgtggaaaagggcatctaccagacctccaacttccgggtgcagcccaccgagtctatcgtgcggttccccaacatcaccaatctgtgtcctttcgacgaggtgttcaatgctacccggtttgcctctgtgtacgcttggaacagaaagagaatctcaaactgcgtggccgactactccgttctgtacaacctggccccattcttcacctttaagtgctacggcgtctctcctaccaagctcaacgacctgtgcttcacaaacgtgtacgccgactctttcgtgattagaggcgacgaagtgagacagatcgctccgggccagaccggaaacatcgccgactacaactacaagctgccagacgacttcaccggctgtgtgatcgcctggaactccaacaagctggactccaaggtgtccggcaattacaactacctgtaccggctgtttcggaagtccaacctgaagcccttcgagcgggatatttccaccgagatctaccaggccggcaacaagccttgtaacggcgttgctggcttcaactgctactttcctctgaagtcctatagcttcaggcctacttacggagtgggccaccagccttacagagtggtcgtgctgtcttttgagctgctgcatgcccctgctacagtctgcggacctaagaaaagcaccaatctggtgaagaacaagtgcgtgaacttcaacttcaatggcctgaaaggcacaggcgtgctgaccgagtctaacaagaagttcctgcccttccagcagttcggtcgggacatagctgacaccaccgatgccgtgcgggaccctcagaccctggaaatcctggatatcaccccttgctccttcgggggagtttccgtgatcacccctggcaccaacacctcaaaccaggtggctgtgctgtatcaaggcgttaactgtaccgaagtgcccgtggccattcacgctgaccagctgaccccaacctggcgggtttactccaccggctcgaacgtgttccagaccagagccggctgcctgatcggcgccgagtacgtcaacaattcttacgagtgtgacatccccatcggcgccggaatctgtgcctcctaccagacacagaccaagtctcatggcggggcctcttccgtggctagccagtccatcatcgcctacaccatgtccctgggagccgaaaactctgtggcctactctaacaactccatcgctatccctactaactttaccatctccgtgaccacagagatcctgcctgtctccatgaccaagacctctgtggactgcactatgtacatctgcggcgactccaccgagtgctctaatctgctgctgcaatatggctccttctgcacccagctgaaacgggctctgaccggcatcgccgtggaacaggacaagaacacacaggaagtgttcgcccaggtgaagcagatctacaagacccctccaatcaaatacttcggcgggttcaacttctcccagattctgcctgatccttctaagcctagcaagcggtcctttatcgaggatctgctgttcaacaaagttacactcgccgatgccggcttcatcaagcagtacggcgactgcctcggagatatcgccgccagagacctgatctgcgctcagaagttcaagggcctgacggtgctgcctcctctcctgaccgatgagatgatcgcccagtacacctctgccctgctggctggcaccatcaccagcggatggaccttcggcgctggcgctgccctacagatccctttcgccatgcagatggcctaccggtttaacggcatcggcgtgacccagaacgtgctgtacgagaaccagaagctgatcgcgaaccagttcaactccgcgatcggcaagatccaggactctctgtccagtaccgcaagtgcactcggcaagctgcaggatgtggtgaaccacaacgcccaggctctgaacaccctcgtgaagcaactgtctagcaagttcggcgccatctcttctgttctgaacgatatcttctcgcggctcgacccacctgaggccgaggtgcaaatcgaccggctgatcaccggcagactgcagtccctgcaaacttacgtgacccagcagctaatcagagctgccgaaatccgtgcatctgccaacctggctgctaccaagatgtcggaatgcgtgctgggccagtctaagagagtggacttctgtggcaagggctaccacctgatgtccttccctcaatctgctcctcacggcgttgtctttctgcatgtgacctatgtgcctgcccaggagaagaacttcacgacagctcctgctatctgtcacgacggcaaggcccactttcctagagaaggcgtgttcgtgtctaatggaacccactggttcgtgacccaaagaaacttctacgaaccccagatcatcacaaccgacaacaccttcgtctccggcaactgcgacgtggtgatcggcatcgtgaataacaccgtgtacgaccctctccagcctgaactggatagcttcaaagaggaactggataagtacttcaagaaccacacttctcctgatgtggacctgggcgacatctccggcatcaacgccagcgtggtgaacatccagaaagaaatcgacagactgaacgaggtggccaagaacctgaacgagtctctgatcgacctgcaggagctgggcaagtacgagcagggtacctacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctacccatacgatgttccagattacgcttaa

SEQ ID NO: 22 - protein, 1268 aa, synthetic construct
  REGION 1..1268: note = FL-S-HA (Omicron)
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGGASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTYIKWPWYIWLGFIAGLIAIVMVTIMLCCMTSCCSCLKGCCSCGSCCKFDEDDSEPVLKGVKLHYTYPYDVPDYA

SEQ ID NO: 23 - DNA, 3855 bp, synthetic construct
  misc_feature 1..3855: note = FL-S-HA stabilizing prolines reverse mutant coding sequence
  source 1..3855: mol_type = other DNA
atgttcgtgtttctggtgctgctgccactggtgtccagccagtgcgtgaacctgaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcaggacgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacaaggtggaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagggtacctacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctacccatacgatgttccagattacgcttaa

SEQ ID NO: 24 - protein, 1271 aa, synthetic construct
  REGION 1..1271: note = FL-S-HA stabilizing prolines reverse mutant
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTYIKWPWYIWLGFIAGLIAIVMVTIMLCCMTSCCSCLKGCCSCGSCCKFDEDDSEPVLKGVKLHYTYPYDVPDYA

SEQ ID NO: 25 - DNA, 3855 bp, synthetic construct
  misc_feature 1..3855: note = FL-S-HA furin cleavage site reverse mutant coding sequence
  source 1..3855: mol_type = other DNA
atgttcgtgtttctggtgctgctgccactggtgtccagccagtgcgtgaacctgaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcaggacgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaaggagggctaggagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagggtacctacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagttcgacgaggacgatagcgaacccgtgctgaaaggcgtgaagctgcactacacctacccatacgatgttccagattacgcttaa

SEQ ID NO: 26 - protein, 1271 aa, synthetic construct
  REGION 1..1271: note = FL-S-HA furin cleavage site reverse mutant
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGTYIKWPWYIWLGFIAGLIAIVMVTIMLCCMTSCCSCLKGCCSCGSCCKFDEDDSEPVLKGVKLHYTYPYDVPDYA

SEQ ID NO: 27 - DNA, 39 bp, SARS-CoV-2 virus
  source 1..39: mol_type = genomic DNA
atgtttgtttttcttgttttattgccactagtctctagt

SEQ ID NO: 28 - DNA, 39 bp, synthetic construct
  misc_feature 1..39: note = Codon-optimized coding sequence for S signal peptide
  source 1..39: mol_type = other DNA
atgttcgtgtttctggtgctgctgccactggtgtccagc

SEQ ID NO: 29 - DNA, 39 bp, synthetic construct
  misc_feature 1..39: note = Codon-optimized coding sequence for S signal peptide
  source 1..39: mol_type = other DNA
atgtttgtgtttctagtgctactaccactggtgagctct

SEQ ID NO: 30 - protein, 13 aa, SARS-CoV-2 virus
MFVFLVLLPLVSS

SEQ ID NO: 31 - DNA, 24 bp, Influenza A virus
  source 1..24: mol_type = genomic DNA
ccatacgatgttccagattacgct

SEQ ID NO: 32 - protein, 9 aa, Influenza A virus
YPYDVPDYA

SEQ ID NO: 33 - protein, 75 aa, SARS-CoV-2 virus
MYSFVSEETGTLIVNSVLLFLAFVVFLLVTLAILTALRLCAYCCNIVNVSLVKPSFYVYSRVKNLNSSRVPDLLV

SEQ ID NO: 34 - protein, 419 aa, SARS-CoV-2 virus
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 35 - protein, 222 aa, SARS-CoV-2 virus
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 36 - protein, 18 aa, SARS-CoV-2 virus
FDEDDSEPVLKGVKLHYT

SEQ ID NO: 37 - protein, 565 aa, Influenza A virus
MKANLLVLLCALAAADADTICIGYHANNSTDTVDTVLEKNVTVTHSVNLLEDSHNGKLCRLKGIAPLQLGKCNIAGWLLGNPECDPLLPVRSWSYIVETPNSENGICYPGDFIDYEELREQLSSVSSFERFEIFPKESSWPNHNTNGVTAACSHEGKSSFYRNLLWLTEKEGSYPKLKNSYVNKKGKEVLVLWGIHHPPNSKEQQNLYQNENAYVSVVTSNYNRRFTPEIAERPKVRDQAGRMNYYWTLLKPGDTIIFEANGNLIAPMYAFALSRGFGSGIITSNASMHECNTKCQTPLGAINSSLPYQNIHPVTIGECPKYVRSAKLRMVTGLRNNPSIQSRGLFGAIAGFIEGGWTGMIDGWYGYHHQNEQGSGYAADQKSTQNAINGITNKVNTVIEKMNIQFTAVGKEFNKLEKRMENLNKKVDDGFLDIWTYNAELLVLLENERTLDFHDSNVKNLYEKVKSQLKNNAKEIGNGCFEFYHKCDNECMESVRNGTYDYPKYSEESKLNREKVDGVKLESMGIYQILAIYSTVASSLVLLVSLGAISFWMCSNGSLQCRICI

SEQ ID NO: 38 - protein, 454 aa, Influenza A virus
MNPNQKIITIGSICLVVGLISLILQIGNIISIWISHSIQTGSQNHTGICNQNIITYKNSTWVKDTTSVILTGNSSLCPIRGWAIYSKDNSIRIGSKGDVFVIREPFISCSHLECRTFFLTQGALLNDRHSNGTVKDRSPYRALMSCPVGEAPSPYNSRFESVAWSASACHDGMGWLTIGISGPDNGAVAVLKYNGIITETIKSWRKKILRTQESECACVNGSCFTIMTDGPSDGLASYKIFKIEKGKVTKSIELNAPNSHYEECSCYPDTGKVMCVCRDNWHGSNRPWVSFDQNLDYQIGYICSGVFGDNPRPKDGTGSCGPVYVDGANGVKGFSYRYGNGVWIGRTKSHSSRHGFEMIWDPNGWTETDSKFSVRQDVVAMTDWSGYSGSFVQHPELTGLDCIRPCFWVELIRGRPKEKTIWTSASSISFCGVNSDTVDWSWPDGAELPFTIDK

SEQ ID NO: 39 - protein, 574 aa, Respiratory syncytial virus
MELPILKANAITTILAAVTFCFASSQNITEEFYQSTCSAVSKGYLSALRTGWYTSVITIELSNIKENKCNGTDAKVKLMKQELDKYKNAVTELQLLMQSTPAANNRARRELPRFMNYTLNNTKKTNVTLSKKRKRRFLGFLLGVGSAIASGIAVSKVLHLEGEVNKIKSALLSTNKAVVSLSNGVSVLTSKVLDLKNYIDKQLLPIVNKQSCRISNIETVIEFQQKNNRLLEITREFSVNVGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 40 - protein, 479 aa, Homo sapiens
MANFTPVNGSSGNQSVRLVTSSSHNRYETVEMVFIATVTGSLSLVTVVGNILVMLSIKVNRQLQTVNNYFLFSLACADLIIGAFSMNLYTVYIIKGYWPLGAVVCDLWLALDYVVSNASVMNLLIISFDRYFCVTKPLTYPARRTTKMAGLMIAAAWVLSFVLWAPAILFWQFVVGKRTVPDNQCFIQFLSNPAVTFGTAIAAFYLPVVIMTVLYIHISLASRSRVHKHRPEGPKEKKAKTLAFLKSPLMKQSVKKPPPGEAAREELRNGKLEEAPPPALPPPPRPVADKDTSNESSSGSATQNTKERPATELSTTEATTPAMPAPPLQPRALNPASRWSKIQIVTKQTGNECVTAIEIVPATPAGMRPAANVARKFASIARNQVRKKRQMAARERKVTRTIFAILLAFILTWTPYNVMVLVNTFCQSCIPDTVWSIGYWLCYVNSTINPACYALCNATFKKTFRHLLLCQYRNIGTAR

SEQ ID NO: 41 - protein, 387 aa, Homo sapiens
MNVSGCPGAGNASQAGGGGGWHPEAVIVPLLFALIFLVGTVGNTLVLAVLLRGGQAVSTTNLFILNLGVADLCFILCCVPFQATIYTLDGWVFGSLLCKAVHFLIFLTMHASSFTLAAVSLDRYLAIRYPLHSRELRTPRNALAAIGLIWGLSLLFSGPYLSYYRQSQLANLTVCHPAWSAPRRRAMDICTFVFSYLLPVLVLGLTYARTLRYLWRAVDPVAAGSGARRAKRKVTRMILIVAALFCLCWMPHHALILCVWFGQFPLTRATYALRILSHLVSYANSCVNPIVYALVSKHFRKGFRTICAGLLGRAPGRASGRVCAAARGTHSGSVLERESSDLLHMSEAAGALRPCPGASQPCILEPCPGPSWQGPKAGDSILTVDVA

SEQ ID NO: 42 - protein, 8 aa, synthetic construct
DYKDDDDK

SEQ ID NO: 43 - protein, 9 aa, Influenza A virus
YPYDVPDYA

SEQ ID NO: 44 - protein, 1242 aa, SARS-CoV-2 virus
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 45 - protein, 1239 aa, SARS-CoV-2 virus
QCVNFTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHISYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPGGASSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 46 - protein, 1240 aa, SARS-CoV-2 virus
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGGASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 47 - protein, 1239 aa, SARS-CoV-2 virus
QCVNLTTRTQLPPAYTNSFTRGVYYPDKVFRSSVLHSTQDLFLPFFSNVTWFHVISGTNGTKRFDNPVLPFNDGVYFASIEKSNIIRGWIFGTTLDSKTQSLLIVNNATNVVIKVCEFQFCNDPFLDHKNNKSWMESEFRVYSSANNCTFEYVSQPFLMDLEGKQGNFKNLREFVFKNIDGYFKIYSKHTPIIVREPEDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFDEVFNATRFASVYAWNRKRISNCVADYSVLYNLAPFFTFKCYGVSPTKLNDLCFTNVYADSFVIRGDEVRQIAPGQTGNIADYNYKLPDDFTGCVIAWNSNKLDSKVSGNYNYLYRLFRKSNLKPFERDISTEIYQAGNKPCNGVAGFNCYFPLKSYSFRPTYGVGHQPYRVVVLSFELLHAPATVCGPKKSTNLVKNKCVNFNFNGLKGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHGGASSVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKRSFIEDLLFNKVTLADAGFIKQYGDCLGDIAARDLICAQKFKGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDIFSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCK

SEQ ID NO: 48 - protein, 5 aa, SARS-CoV-2 virus
KLHYT

SEQ ID NO: 49 - DNA, 3774 bp, SARS-CoV-2 virus
  source 1..3774: mol_type = other DNA
atgttcgtgtttctggtgctgctgccactggtgtccagccagtgcgtgaacctgaccacaagaacccagctgccccctgcctataccaattctttcacaaggggcgtgtactatcccgacaaggtgtttcggtcttccgtgctgcactccacacaggatctgtttctgcctttctttagcaacgtgacctggttccacgctatccacgtgtccggcaccaatggcacaaagaggttcgacaatccagtgctgccctttaacgatggcgtgtacttcgcctccaccgagaagagcaacatcatccggggctggatctttggcaccacactggactctaagacacagtccctgctgatcgtgaacaatgctaccaacgtggtcatcaaggtgtgcgagttccagttttgtaatgatccattcctgggcgtgtactatcataagaacaataagagctggatggagtctgagtttcgcgtgtatagctctgccaacaattgtacatttgagtacgtgtctcagcccttcctgatggacctggagggcaagcagggcaatttcaagaacctgagagagttcgtgtttaagaatatcgatggctacttcaagatctactccaagcacaccccaatcaacctggtgcgcgacctgccacagggcttcagcgccctggagccactggtggatctgcccatcggcatcaacatcaccaggtttcagacactgctggccctgcatcggtcctacctgacaccaggcgactccagctctggatggaccgctggagctgctgcctactatgtgggctatctgcagcccagaaccttcctgctgaagtacaacgagaatggcaccatcacagacgctgtggattgcgccctggaccccctgagcgagaccaagtgtacactgaagtcttttaccgtggagaagggcatctatcagacatctaatttcagagtgcagcctaccgagtccatcgtgcgctttcccaatatcacaaacctgtgcccttttggcgaggtgttcaacgctacccgcttcgccagcgtgtacgcttggaatagaaagcgcatctccaactgcgtggccgactatagcgtgctgtacaactccgctagcttctctacctttaagtgctatggcgtgtctcccacaaagctgaatgacctgtgctttaccaacgtgtacgctgattccttcgtgatcagaggcgacgaggtgcgccagatcgctccaggacagacaggcaagatcgctgactacaattataagctgcctgacgatttcaccggctgcgtgatcgcctggaacagcaacaatctggattctaaagtgggcggcaactacaattatctgtacaggctgtttcggaagagcaatctgaagccattcgagagagacatctccacagagatctaccaggctggcagcaccccatgcaatggagtggagggctttaactgttatttccctctgcagtcttacggcttccagccaaccaacggcgtgggctatcagccctaccgcgtggtggtgctgagctttgagctgctgcacgctcctgctacagtgtgcggaccaaagaagtctaccaatctggtgaagaacaagtgcgtgaacttcaacttcaacggactgaccggaacaggcgtgctgaccgagagcaacaagaagttcctgccttttcagcagttcggcagagacatcgccgataccacagacgctgtgcgcgaccctcagaccctggagatcctggatatcacaccatgctccttcggcggcgtgagcgtgatcacaccaggcaccaatacatctaaccaggtggccgtgctgtatcaggacgtgaattgtaccgaggtgcccgtggctatccacgccgatcagctgacccctacatggagggtgtactctaccggctccaacgtgtttcagacaagggctggatgtctgatcggagctgagcatgtgaacaattcttatgagtgcgacatccctatcggcgccggcatctgtgcttcctaccagacccagacaaactccccaggaggagctagcagcgtggcttcccagagcatcatcgcctataccatgtctctgggcgccgagaactccgtggcttacagcaacaattctatcgctatccctaccaacttcacaatctccgtgaccacagagatcctgccagtgtctatgaccaagacatccgtggactgcacaatgtatatctgtggcgattccaccgagtgcagcaacctgctgctgcagtacggcagcttttgtacccagctgaatcgggccctgacaggcatcgctgtggagcaggacaagaacacacaggaggtgttcgcccaggtgaagcagatctacaagaccccacccatcaaggactttggcggcttcaacttctcccagatcctgcccgatccttccaagcccagcaagaggtcttttatcgaggacctgctgttcaacaaggtgaccctggctgatgccggcttcatcaagcagtatggcgattgcctgggcgacatcgctgctagggacctgatctgtgcccagaagtttaatggcctgaccgtgctgcctccactgctgacagatgagatgatcgctcagtacacatccgccctgctggccggcaccatcacatctggatggaccttcggcgctggagctgccctgcagatcccttttgccatgcagatggcttatcggttcaacggcatcggcgtgacccagaatgtgctgtacgagaaccagaagctgatcgccaatcagtttaactccgctatcggcaagatccaggacagcctgtccagcacagcttctgccctgggcaagctgcaggatgtggtgaatcagaacgctcaggccctgaataccctggtgaagcagctgtcttccaacttcggcgccatcagctctgtgctgaatgatatcctgagcaggctggacccacctgaggctgaggtgcagatcgacaggctgatcacaggccggctgcagtccctgcagacctatgtgacacagcagctgatcagagctgccgagatccgcgctagcgccaacctggctgccaccaagatgtctgagtgcgtgctgggccagtccaagcgggtggacttttgtggcaagggctatcacctgatgtctttcccacagtccgcccctcacggagtggtgtttctgcatgtgacctacgtgccagctcaggagaagaacttcaccacagctcccgccatctgccacgatggcaaggcccattttcctagagagggcgtgttcgtgtctaacggcacccattggtttgtgacacagcgcaatttctacgagccacagatcatcaccacagacaataccttcgtgtccggcaactgtgacgtggtcatcggcatcgtgaacaataccgtgtatgatcctctgcagccagagctggacagctttaaggaggagctggataagtacttcaagaatcacacatctcctgacgtggatctgggcgacatcagcggcatcaatgcctctgtggtgaacatccagaaggagatcgataggctgaacgaggtggctaagaatctgaacgagtctctgatcgacctgcaggagctgggcaagtatgagcagggtacctacatcaagtggccttggtacatctggctgggcttcatcgctggcctgatcgccatcgtgatggtcacaatcatgctgtgctgcatgacctcctgctgttcttgcctgaagggctgctgctcctgtggatcttgttgcaagtaa

(19) United States
(12) Patent Application Publication
(10) Pub. No.: US 2026/0014249 A1
(43) Pub. Date: Jan. 15, 2026

(54) ENVELOPED VIRUS LIKE PARTICLES COMRISING SARS-COV-2 S PROTEIN

(71) Applicant: National Research Council of Canada, Ottawa (CA)

(72) Inventors: Sergio P. ALPUCHE-LAZCANO, La Prairie (CA); Matthew STUIBLE, Mont-Royal (CA); Yves DUROCHER, Montreal (CA); Zalma Vanesa SANCHEZ MARTINEZ, Montreal (CA)

(73) Assignee: National Research Council of Canada, Ottawa, ON (CA)

(21) Appl. No.: 18/995,964

(22) PCT Filed: Jul. 17, 2023
(86) PCT No.: PCT/IB2023/057279

§ 371 Date: Jan. 17, 2025

Related U.S. Application Data
(60) Provisional application No. 63/368,685, filed on Jul. 18, 2022.
(60) Provisional application No. 63/497,284, filed on Apr. 20, 2023.

(51) Int. Cl. A61K 39/215; A61K 39/00; A61K 39/145; A61P 31/14; A61P 37/04; C07K 14/005; C12N 15/86

(52) U.S. Cl. CPC A61K 39/215; A61K 39/145; A61P 31/14; A61P 37/04; C07K 14/005; C12N 15/86; A61K 2039/5258; A61K 2039/55572; A61K 2039/55577

(57) ABSTRACT

Provided is an enveloped virus-like particle (eVLP) comprising a substantially full-length recombinant SARS-CoV-2 spike (S) protein. The eVLP may further comprise an additional recombinant SARS-CoV-2 S protein having a different sequence, another recombinant viral antigen, or a recombinant non-viral protein. The eVLP is derived from an animal cell, such as a CHO cell, expressing the recombinant SARS-CoV-2 spike protein. Also provided are methods of producing such eVLPs, compositions including such eVLPs, and methods and uses for the induction of an immune response against a SARS-CoV-2 spike protein and/or prevention of COVID-19 or SARS-CoV-2 infection, employing such eVLPs.

### CROSS-REFERENCE TO RELATED APPLICATIONS

This application claims the benefit of priority from U.S. provisional patent application No. 63/497,284, filed Apr. 20, 2023 and U.S. provisional patent application No. 63/368,685, filed Jul. 18, 2022, each of which is herein incorporated by reference in its entirety.

### REFERENCE TO AN ELECTRONIC SEQUENCE LISTING

The contents of the electronic sequence listing (2022-023-03_SL.xml; Size: 124,840 bytes; and Date of Creation: Jul. 14, 2023) is herein incorporated by reference in its entirety.

### FIELD

The present disclosure relates to enveloped virus-like particles (eVLPs) comprising SARS-CoV-2 spike protein(S) and compositions comprising the same. The present disclosure further relates to the use of such eVLPs or compositions to induce an immune response against SARS-CoV-2 S and/or another antigen that is included in the eVLP.

### BACKGROUND

SARS-CoV-2 is a highly transmissible coronavirus that causes the respiratory disease “coronavirus disease 2019” (COVID-19). SARS-CoV-2 first emerged in late 2019 and rapidly spread to cause a global pandemic, eliciting a global effort to develop safe and effective vaccines. An array of antigen production and delivery strategies have been explored in the development of SARS-CoV-2 vaccines. Most approved vaccines are based on mRNA- or viral vector-induced expression of spike protein(S) antigen by host cells (Pecetta et al, 2022). Purified antigen vaccines have been generally slower to develop, but could offer advantages in the long-term, global response to COVID-19. Protein subunit vaccines consisting of the full ectodomain or fragments of the S protein have been evaluated extensively (Martinez-Flores et al, 2021). To mimic the trimeric structure of native S on the virus surface, the S ectodomain is often fused to a trimerization domain, such as T4 foldon. Potential advantages of virus-like particles (VLPs) over other technologies include improved storage stability, cell uptake, and Th1/Th2 responses. VLPs also eliminate the risk of revertant strains associated with attenuated virus-based strategies (Fuenmayor & Cervera et al, 2017; Plotkin et al, 2014).

VLPs appear increasingly well-regarded as platforms for displaying S antigens for clinical SARS-CoV-2 vaccine development. VBI Vaccines completed Phase I trials with VLPs produced by retroviral Gag co-expression with S in HEK293 cells (Fluckiger et al, 2021), and Medicago-GlaxoSmithKline achieved regulatory approval after completing Phase III trials using VLPs produced in plants. Medicago's VLP technology is based on a fusion of the S ectodomain with the transmembrane and C-terminal tails of influenza HA (D'Aoust et al, 2008; Ward et al, 2021). VLPs can also be produced by co-expression of the coronavirus structural proteins, membrane (M), envelope (E), nucleocapsid (N) and S, an approach more closely reflecting the normal process of virus assembly and which is being pursued to clinical testing (Yilmaz et al, 2022). There is also interest in producing VLPs incorporating other antigens (viral or non-viral) in order to make multivalent vaccines.

There is a desire for VLPs displaying the SARS-CoV-2 S antigen that may be readily produced and that elicit a robust immune response.

### SUMMARY

The present inventors have found that enveloped virus-like particles (eVLPs) can be produced by expressing a substantially full-length recombinant SARS-CoV-2 spike protein (FL-S) in an animal host cell, such as but not limited to a mammalian cell such as a Chinese hamster ovary (CHO) cell. The host cell then releases eVLPs comprising FL-S into a surrounding medium, from which the eVLPs can be isolated or purified. The eVLP may comprise a single FL-S variant (monovariant eVLP) or it may comprise two or more FL-S variants (multivariant eVLP). The eVLP may further comprise one or more additional recombinant proteins other than FL-S (multivalent eVLP).

Accordingly, there is provided an enveloped virus-like particle (eVLP) derived from an animal host cell, wherein the eVLP comprises a substantially full-length recombinant SARS-CoV-2 spike protein. In an embodiment, the host cell is a mammalian cell. In a further embodiment, the host cell is a Chinese hamster ovary (CHO) cell.

In an embodiment, the substantially full-length recombinant SARS-CoV-2 spike protein comprises a mutation that reduces or eliminates the function of the endoplasmic reticulum (ER) retention signal of the spike protein. In a further embodiment, the mutation that reduces or eliminates the function of the ER retention signal is a C-terminal truncation of 5 to 21 contiguous amino acids relative to the full-length SARS-CoV-2 spike protein sequence.

In an embodiment, the substantially full-length recombinant SARS-CoV-2 spike protein is a chimeric protein further comprising a heterologous polypeptide fused to the C-terminus of the SARS-CoV-2 spike protein sequence.

In an embodiment, the substantially full-length recombinant SARS-CoV-2 spike protein is stabilized in the prefusion conformation.

In an embodiment, the substantially full-length recombinant SARS-CoV-2 spike protein comprises an inactivated S1/S2 furin cleavage site.

In an embodiment, the substantially full-length recombinant SARS-CoV-2 spike protein comprises an amino acid sequence having at least 70% sequence identity to the full length of the amino acid sequence set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47.

In an embodiment, the substantially full-length recombinant SARS-CoV-2 spike protein comprises an amino acid sequence as set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47, except that the amino acid sequence comprises between one and 60 amino acid substitutions, insertions, and/or deletions relative to the amino acid sequence set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47.

In an embodiment, the substantially full-length recombinant SARS-CoV-2 spike protein comprises an amino acid sequence as set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47, except that the amino acid sequence comprises between one and 20 conservative amino acid substitutions relative to the amino acid sequence set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47.

In an embodiment, the eVLP does not comprise one or more of: SARS-CoV-2 envelope protein, SARS-CoV-2 membrane protein, and SARS-CoV-2 nucleocapsid protein. In an embodiment, the eVLP does not comprise SARS-CoV-2 envelope protein or SARS-CoV-2 membrane protein. In another embodiment, the eVLP comprises neither SARS-CoV-2 envelope protein nor SARS-CoV-2 membrane protein. In an embodiment, the eVLP comprises none of SARS-CoV-2 envelope protein, SARS-CoV-2 membrane protein, and SARS-CoV-2 nucleocapsid protein.

In an embodiment, the substantially full-length recombinant SARS-CoV-2 spike protein makes up at least 50% of the total protein content of the eVLP. In other embodiments, the substantially full-length recombinant SARS-CoV-2 spike protein makes up at least 50%, at least 55%, at least 60%, at least 65%, at least 70%, at least 75%, or at least 80% of the total protein content of the eVLP.

In an embodiment, the eVLP is a multivalent eVLP further comprising an additional recombinant protein. In an embodiment, the additional recombinant protein comprises a non-viral recombinant protein. In a further embodiment, the additional recombinant protein comprises a cell surface protein. In another embodiment, the additional recombinant protein comprises a viral antigen from a virus other than SARS-CoV-2. In an embodiment, the additional recombinant protein comprises an influenza A antigen. In a further embodiment, the additional recombinant protein comprises influenza hemagglutinin or neuraminidase. In an embodiment, the additional recombinant protein comprises both influenza hemagglutinin and neuraminidase. In an embodiment, the additional recombinant protein comprises respiratory syncytial virus (RSV) fusion (F) glycoprotein. In another embodiment, the additional recombinant protein comprises human muscarinic acetylcholine receptor M4 or GALR2. In embodiments, the multivalent eVLP is a bivalent eVLP comprising one additional recombinant protein, a trivalent eVLP comprising two additional recombinant proteins, or a tetravalent eVLP comprising three additional recombinant proteins.

In an embodiment, the substantially full-length recombinant SARS-CoV-2 spike protein makes up at least 25% of the total protein content of the multivalent eVLP. In other embodiments, the substantially full-length recombinant SARS-CoV-2 spike protein makes up at least 20%, at least 25%, at least 30%, at least 35%, at least 40%, at least 45%, at least 50%, at least 55%, or at least 60% of the total protein content of the multivalent eVLP.

In an embodiment, the eVLP does not comprise any recombinant protein, other than the substantially full-length recombinant SARS-CoV-2 spike protein, that is substantially capable of independently inducing eVLP formation by the host cell. In a further embodiment, the eVLP does not comprise any of: retroviral Gag protein, influenza matrix protein, SARS-CoV-2 envelope protein, SARS-CoV-2 membrane protein, hepatitis B surface antigen, and Ebola virus VP40.

In an embodiment, the eVLP is a multivariant eVLP comprising at least one additional substantially full-length recombinant SARS-CoV-2 spike protein having a different amino acid sequence from the substantially full-length recombinant SARS-CoV-2 spike protein.

In an embodiment, the multivariant eVLP does not comprise any recombinant protein, other than the substantially full-length recombinant SARS-CoV-2 spike protein and the at least one additional substantially full-length recombinant SARS-CoV-2 spike protein, that is substantially capable of independently inducing eVLP formation by the host cell. In a further embodiment the multivariant eVLP does not comprise any of: retroviral Gag protein, influenza matrix protein, SARS-CoV-2 envelope protein, SARS-CoV-2 membrane protein, hepatitis B surface antigen, and Ebola virus VP40.

In an embodiment, the substantially full-length recombinant SARS-CoV-2 spike protein and the at least one additional substantially full-length recombinant SARS-CoV-2 spike protein together make up at least 50% of the total protein content of the multivariant eVLP. In other embodiments, the substantially full-length recombinant SARS-CoV-2 spike protein and the at least one additional substantially full-length recombinant SARS-CoV-2 spike protein together make up at least 50%, at least 55%, at least 60%, at least 65%, at least 70%, at least 75%, or at least 80% of the total protein content of the multivariant eVLP.

In an embodiment, the multivariant eVLP is also multivalent and further comprises an additional recombinant protein, as described herein.

In an embodiment, an eVLP as described herein is isolated or purified.

In an embodiment, an eVLP as described herein has a diameter of about 50 nm to about 150 nm.

Another aspect of the disclosure is a composition comprising a plurality of eVLPs as described herein, wherein the eVLPs in the composition have a median diameter of about 115 to about 135 nm.

Yet another aspect of the disclosure is a method for preparing an enveloped virus-like particle (eVLP), the method comprising:
a) providing an animal host cell comprising within its nucleus a nucleic acid molecule encoding a substantially full-length recombinant SARS-CoV-2 spike protein;
b) incubating the host cell in a medium under conditions that allow the substantially full-length recombinant SARS-CoV-2 spike protein to be expressed by the host cell; and
c) allowing the host cell to produce the eVLP and release the eVLP into the medium,
wherein the substantially full-length recombinant SARS-CoV-2 spike protein is as defined herein.

In an embodiment of the method, the host cell is a mammalian host cell. In a further embodiment, the mammalian cell is a CHO cell. In a still further embodiment, the CHO cell comprises a mutation that inactivates a functional endogenous retrovirus sequence in its genome.

In an embodiment, the method further comprises isolating the eVLP from the medium.

In an embodiment, the method results in the production of at least 1.0×10¹² eVLPs per liter of culture. In another embodiment, the method results in the production of at least 1.0×10¹³ eVLPs per liter of culture. In other embodiments, the method results in the production and purification of at least 1.0×10¹² or at least 1.0×10¹³ eVLPs per liter of culture.

In an embodiment, the method results in the production of a plurality of eVLPs having a median diameter of about 115 to about 135 nm.

In an embodiment of the method, step (a) comprises transfecting the host cell with the nucleic acid molecule. In a further embodiment, the nucleic acid molecule is a plasmid.

In an embodiment of the method, the nucleic acid molecule comprises a nucleotide sequence encoding the substantially full-length recombinant SARS-CoV-2 spike protein operatively linked to a heterologous regulatory element that is operative in the host cell to allow the substantially full-length recombinant SARS-CoV-2 spike protein to be expressed by the host cell.

In an embodiment of the method, the nucleic acid molecule is codon-optimized for expression in the host cell. In a further embodiment, the nucleic acid molecule comprises the nucleotide sequence set forth in SEQ ID NO: 3, SEQ ID NO: 6, SEQ ID NO: 9, SEQ ID NO: 12, SEQ ID NO: 15, SEQ ID NO: 17, SEQ ID NO: 19, SEQ ID NO: 21, SEQ ID NO: 23, or SEQ ID NO: 49.

In an embodiment of the method, the substantially full-length SARS COV-2 spike protein makes up at least 50% of the total protein content of the eVLP produced by the method. In other embodiments, the substantially full-length SARS COV-2 spike protein makes up at least 50%, at least 55%, at least 60%, at least 65%, at least 70%, at least 75%, or at least 80% of the total protein of the eVLP produced by the method.

In an embodiment, the method is for production of a multivariant eVLP and the host cell comprises within its nucleus a nucleic acid molecule encoding at least one additional substantially full-length recombinant SARS-CoV-2 spike protein having a different amino acid sequence than the substantially full-length recombinant SARS-CoV-2 spike protein. In a further embodiment, the substantially full-length recombinant SARS-CoV-2 spike protein and the at least one additional substantially full-length recombinant SARS-CoV-2 spike protein together make up at least 50% of the total protein content of the multivariant eVLP produced by the method. In other embodiments, the substantially full-length recombinant SARS-CoV-2 spike protein and the at least one additional substantially full-length recombinant SARS-CoV-2 spike protein together make up at least 50%, at least 55%, at least 60%, at least 65%, at least 70%, at least 75%, or at least 80% of the total protein of the multivariant eVLP produced by the method.

In another embodiment the method is for production of a multivalent eVLP and the host cell provided in step (a) further comprises within its nucleus a nucleic acid molecule encoding an additional recombinant protein operatively linked to a regulatory element that is operative in the cell to allow the additional recombinant protein to be expressed by the cell. In a further embodiment, the additional recombinant protein comprises a non-viral protein. In a further embodiment, the additional recombinant protein comprises a cell surface protein. In another embodiment, the additional recombinant protein comprises a viral antigen other than SARS-CoV-2 S. In a further embodiment, the viral antigen is an influenza A antigen. In a further embodiment, the additional recombinant protein comprises influenza hemagglutinin or neuraminidase. In an embodiment, the additional recombinant protein comprises both influenza hemagglutinin and neuraminidase. In an embodiment, the additional recombinant protein comprises respiratory syncytial virus (RSV) fusion (F) glycoprotein. In another embodiment, the additional recombinant protein comprises human muscarinic acetylcholine receptor M4 or GALR2. In an embodiment, the substantially full-length recombinant SARS-CoV-2 spike protein makes up at least 25% of the total protein content of the multivalent eVLP produced by the method. In other embodiments, the substantially full-length recombinant SARS-CoV-2 spike protein makes up at least 20%, at least 25%, at least 30%, at least 35%, at least 40%, at least 45%, at least 50%, at least 55%, or at least 60% of the total protein content of a multivalent eVLP produced by the method.

In an embodiment of the method, the host cell does not co-express together with the substantially full-length recombinant SARS-CoV-2 spike protein any viral protein, other than the substantially full-length recombinant SARS-CoV-2 spike protein, that is substantially capable of independently inducing eVLP formation by the host cell. In a further embodiment, the host cell does not co-express together with the substantially full-length recombinant SARS-CoV-2 spike protein any of: retroviral Gag protein, influenza matrix protein, SARS-CoV-2 envelope protein, SARS-CoV-2 membrane protein, hepatitis B surface antigen, and Ebola virus VP40.

In an embodiment of the method, the host cell does not co-express together with the substantially full-length recombinant SARS-CoV-2 spike protein one or more of: SARS-CoV-2 envelope protein, SARS-CoV-2 membrane protein, and SARS-CoV-2 nucleocapsid protein. In an embodiment, the host cell does not co-express together with the substantially full-length recombinant SARS-CoV-2 spike protein SARS-CoV-2 envelope protein or SARS-CoV-2 membrane protein. In another embodiment, the host cell does co-expresses together with the substantially full-length recombinant SARS-CoV-2 spike protein neither SARS-CoV-2 envelope protein nor SARS-CoV-2 membrane protein. In an embodiment, the host cell co-expresses together with the substantially full-length recombinant SARS-CoV-2 spike protein none of SARS-CoV-2 envelope protein, SARS-CoV-2 membrane protein, and SARS-CoV-2 nucleocapsid protein.

Another aspect of the disclosure is an animal cell for the production of an enveloped virus-like particle (eVLP) comprising a substantially full-length recombinant SARS-CoV-2 spike protein, wherein the cell comprises within its nucleus a nucleic acid molecule encoding the substantially full-length recombinant SARS-CoV-2 spike protein operatively linked to a heterologous regulatory element that is operative in the cell to allow the substantially full-length recombinant SARS-CoV-2 spike protein to be expressed by the cell, wherein the substantially full-length recombinant SARS-CoV-2 spike protein is as described herein.

In an embodiment, the animal cell is a mammalian cell. In a further embodiment, the mammalian cell is a CHO cell. In a still further embodiment, the CHO cell comprises a mutation that inactivates a functional endogenous retrovirus sequence in its genome.

In an embodiment, the cell is for production of a multivalent eVLP and further comprises within its nucleus a nucleic acid molecule encoding an additional recombinant protein operatively linked to a regulatory element that is operative in the cell to allow the additional recombinant protein to be expressed by the cell. In an embodiment, the additional recombinant protein comprises a non-viral protein. In an embodiment, the additional recombinant protein comprises a cell surface protein. In an embodiment, the additional recombinant protein comprises a viral antigen other than SARS-CoV-2 S. In an embodiment, the additional recombinant protein comprises an influenza A antigen. In an embodiment, the additional recombinant protein comprises influenza hemagglutinin or neuraminidase. In an embodiment, the additional recombinant protein comprises both influenza hemagglutinin and neuraminidase. In an embodiment, the additional recombinant protein comprises respiratory syncytial virus (RSV) fusion (F) glycoprotein. In another embodiment, the additional recombinant protein comprises human muscarinic acetylcholine receptor M4 or GALR2.

In an embodiment, the cell does not comprise any viral protein, other than the substantially full-length recombinant SARS-CoV-2 spike protein, that is capable of independently inducing eVLP formation by the host cell. In a further embodiment, the cell does not comprise any of: retroviral Gag protein, influenza matrix protein, SARS-CoV-2 envelope protein, SARS-CoV-2 membrane protein, hepatitis B surface antigen, and Ebola virus VP40

In an embodiment, the cell does not comprise one or more of: SARS-CoV-2 envelope protein, SARS-CoV-2 membrane protein, and SARS-CoV-2 nucleocapsid protein. In an embodiment, the cell does not comprise SARS-CoV-2 envelope protein or SARS-CoV-2 membrane protein. In another embodiment, the cell comprises neither SARS-CoV-2 envelope protein nor SARS-CoV-2 membrane protein. In an embodiment, the cell comprises none of SARS-CoV-2 envelope protein, SARS-CoV-2 membrane protein, and SARS-CoV-2 nucleocapsid protein.

In an embodiment, the nucleic acid molecule is codon-optimized for expression in the cell. In a further embodiment, the nucleic acid molecule comprises the nucleotide sequence set forth in SEQ ID NO: 3, SEQ ID NO: 6, SEQ ID NO: 9, SEQ ID NO: 12, SEQ ID NO: 15, SEQ ID NO: 17, SEQ ID NO: 19, SEQ ID NO: 21, SEQ ID NO: 23, or SEQ ID NO: 49.

In an embodiment, the cell is for production of a multivariant eVLP and comprises within its nucleus a nucleic acid molecule encoding a second substantially full-length recombinant SARS-CoV-2 spike protein having a different amino acid sequence from the substantially full-length recombinant SARS-CoV-2 spike protein, operatively linked to a heterologous regulatory element that is operative in the cell to allow the second substantially full-length recombinant SARS-CoV-2 spike protein to be expressed by the cell.

Another aspect of the disclosure is use of a cell as described herein to produce an eVLP as described herein comprising the substantially full-length recombinant SARS-CoV-2 spike protein.

Another aspect of the disclosure is a pharmaceutical composition comprising an eVLP as described herein, a composition as described herein, an eVLP produced by a method as described herein, or an eVLP produced by a cell as described herein, and a pharmaceutically acceptable carrier or diluent. In an embodiment, the pharmaceutical composition further comprises an adjuvant. In embodiments, the adjuvant comprises 3-O-desacyl-4′-monophosphoryl lipid A (MPL) and/or a saponin. In an embodiment, the adjuvant comprises MPL and QS-21.

In an embodiment, the pharmaceutical composition is an immunogenic composition or a vaccine composition.

Another aspect of the disclosure is a method of inducing an immune response in a subject, the method comprising administering to the subject: an eVLP as described herein, a composition as described herein, an eVLP produced by a method as described herein, an eVLP produced by a cell as described herein, or a pharmaceutical composition as described herein.

Another aspect of the disclosure is a method for preventing COVID-19 or SARS-CoV-2 infection in a subject, the method comprising administering to the subject: an eVLP as described herein, a composition as described herein, an eVLP produced by a method as described herein, an eVLP produced by a cell as described herein, or a pharmaceutical composition as described herein.

Another aspect of the disclosure is an eVLP as described herein, a composition as described herein, an eVLP produced by a method as described herein, an eVLP produced by a cell as described herein, or a pharmaceutical composition as described herein, for use to induce an immune response in a subject.

Another aspect of the disclosure is an eVLP as described herein, a composition as described herein, an eVLP produced by a method as described herein, an eVLP produced by a cell as described herein, or a pharmaceutical composition as described herein, for use to prevent COVID-19 or SARS-CoV-2 infection.

Another aspect of the disclosure is use of an eVLP as described herein, a composition as described herein, an eVLP produced by a method as described herein, an eVLP produced by a cell as described herein, or a pharmaceutical composition as described herein to induce an immune response in a subject.

Another aspect of the disclosure is use of an eVLP as described herein, a composition as described herein, an eVLP produced by a method as described herein, an eVLP produced by a cell as described herein, or a pharmaceutical composition as described herein to prevent COVID-19 or SARS-CoV-2 infection.

Another aspect of the disclosure is use of an eVLP as described herein, a composition as described herein, an eVLP produced by a method as described herein, an eVLP produced by a cell as described herein, or a pharmaceutical composition as described herein in the preparation of a medicament for the prevention of COVID-19 or SARS-CoV-2 infection.

### BRIEF DESCRIPTION OF DRAWINGS

FIG. 1, upper portion, shows SDS-PAGE with total protein (Coomassie Blue) staining of sedimented (on iodixanol cushion) full length spike protein (FL-S) enveloped virus-like particles (eVLPs). From left to right, the lanes are soluble spike standard (STD) of 1, 0.75, 0.5, 0.25, or 0.125 μg, followed by sedimented and purified FL-S eVLP preparations contained spike concentrations of 0.124 μg/μl and 0.041 μg/μl respectively.

FIG. 2, left panel, shows representative TEM image of affinity-purified FL-S eVLPs. 100 nm scale bar is shown at the bottom. The upper right panel shows a Cryo-EM image of an individual eVLP (50 nm scale). The lower right panel shows a 10-nm scale cropped image of the individual eVLP, in which individual spike proteins embedded in the particle's membrane are visible.

FIG. 3 shows a dot graph of humoral immune response (total anti-spike IgG) after booster shot with FL-S eVLP vaccine formulations for non-adjuvanted, adjuvanted and naïve animals. Anti-spike geometric mean titers (GMT) at day 28 from naïve and treated groups were determined by ELISA. Data are presented as mean±SEM of 10 animals per group. A one-way ANOVA with Tukey's multiple comparison test was performed to assess significance. P values ≤0.0001 are represented with four asterisks.

FIG. 4 shows the results of quantification of the cell response with FL-S eVLP vaccine formulations by IFN-γ+ ELISpot. Data are presented as mean±SEM of 10 animals per group. A one-way ANOVA with Tukey's multiple comparison test was performed to assess significance. P values≤0.0001 are represented with four asterisks.

FIG. 5 shows the results of cell-based (hACE2-HEK293T) surrogate neutralization assays at dilutions of 1:25 (unadjuvanted/Adju-Phos®) or 1:75 (AS01b) for SARS-CoV-2 reference strain (Wuhan). Data are presented as mean±SEM of 10 animals per group. A one-way ANOVA with Dunnett's multiple comparison was performed to assess significance. P≤0.0001 and p=0.0002 are represented with four and three asterisks respectively. P=0.3716 is not significant (NS).

FIG. 6 shows a general schematic of particular SARS-CoV-2 plasmid constructs described herein.

FIG. 7 shows a Western blot and SDS-PAGE using supernatants from CHO55E1 cells transfected with M-E/FL-S-HA or FL-S-HA alone. The left panel shows an immunoblot with HA antibody detecting FL-S-HA (labelled “FL-Spike”, ˜180 kDa) and S2 domain (˜80 kDa). Membrane protein is detected with anti-M at ˜17 kDa. The right panel shows total protein (Coomassie blue) staining of sedimented supernatants.

FIG. 8 shows a representative TEM image of M-E/FL-S eVLPs. White arrows indicate potential VLPs. A 500 nm scale bar is shown at the bottom.

FIG. 9 shows a representative TEM image of FL-S eVLPs. eVLPs in this image average ˜130 nm in diameter with protruded spike proteins of ˜23 nm in length.

FIG. 10 shows representative TEM images and immunoblots of different variant of concern (VOC) FL-S eVLPs. From top to bottom, FIG. 10 shows TEM images of sedimented mock, beta, delta and omicron FL-S eVLPs. White arrows within images indicate eVLPs. A 200 or 100 nm scale bar is shown at the bottom of each image.

FIG. 11 shows a representative Western blot of VOC eVLPs. The immunoblot is stained with anti-HA.

FIG. 12 shows production of FL-S eVLPs in CHO-C2 (endogenous retrovirus-like particle (RVLP) CRISPR knockout (KO)) and parental CHO cells. On the left, SDS-PAGE with total protein staining (Coomassie) shows bulk and sedimented eVLPs produced in CHO²³⁵³ (parental) or CHO-C2 (RVLP KO). On the right, Western blotting of eVLPs produced in CHO2353 or CHO-C2. The upper part of the blot shows the FL-S-HA protein detected with anti-spike (S1) (ProSci #9083) in bulk or sedimented eVLPs. The lower blot was stained with anti-Gag p30 (homemade) demonstrating the absence of Gag in CHO-C2.

FIG. 13 shows the results of eVLP stability analysis. FL-S eVLPs were tracked and evaluated for 101 days. The left Y-axis (circles) shows particle concentration determination (VLPs/ml) by nanoparticle tracking analysis (NTA). The right Y-axis (inverted triangles) shows spike concentration (μg/μL) determined by the curve of soluble spike standards. Protein and particle concentrations in the graph represent the average of different samples.

FIG. 14 shows analysis of hACE2-Vero E6 cells overexpressing human ACE2, which were co-incubated with soluble spike protein and sera/plasma of mice immunized with FL-S eVLP vaccine formulations (surrogate neutralization assay) at dilutions of 1:75. The upper panel shows a reference (Wuhan S) while the middle and lower panels show variants of concern, with the middle panel showing Beta S and the lower panel showing Delta S. Data are presented as mean±SEM of 5 animals per group. A one-way ANOVA with Dunnett's multiple comparison test was performed to assess significance. P values <0.0001 are indicated with four asterisks and p=0.0366 is represented with a single asterisk. Differences with p values >0.05 are considered NS.

FIG. 15, left panel, shows SDS-PAGE with total protein (Coomassie Blue) staining of sedimented FL-S eVLPs showing (from left to right lanes) 1) FL-S eVLPs containing mutations on the spike protein at the S1/S2 furin cleavage site 682 (RRAR>GGAS) with stabilizing prolines (986 KV>PP). 2) Stabilizing prolines reverse mutant of the spike, 682 (RRAR>GGAS)+ (986 KV) and 3) furin cleavage site reverse mutant of the spike, 682 (RRAR)+986 (KV>PP). The right panel shows a FL-S eVLP immunoblot showing (from left to right lanes) 1) regular FL-S VLPs, 2) stabilizing prolines reverse mutant and 3) furin cleavage site reverse mutant. Spike protein was stained using anti-spike (S1) (ProSci #9083).

FIG. 16 shows TEM images of sedimented FL-S reverse mutant eVLPs. The left panel shows a representative TEM image of a stabilizing prolines reverse mutant, 682 (RRAR>GGAS)+ (986 KV). 400 nm scale bar is shown at the bottom. The right panel shows a representative TEM image of a furin cleavage site reverse mutant, 682 (RRAR)+986 (KV>PP). 200 nm scale bar is shown at the bottom. White arrows within images indicate eVLPs.

FIG. 17 shows a schematic of the structure of the SARS-CoV-2 spike protein, illustrating the location of the signal peptide (aa 1-13), the S1 subunit (aa 14-685) and the S2 subunit (aa 686-1273), which includes the transmembrane domain (TM) (aa 1213-1237), and the C-terminal tail (CT) (aa 1237-1273). Sequence numbering is relative to the reference SARS-CoV-2 spike protein provided in SEQ ID NO: 2.

FIG. 18 shows a dot graph of total anti-spike IgG produced by immunized mice on day 20 after a single shot with FL-S eVLP or the trimeric spike protein (ECDm-T4-Fib) vaccine formulations for non-adjuvanted (Ag only), adjuvanted (with Adju-Phos® or AS01b) and naïve animals. Black circles represent FL-S eVLP IgG levels whereas inverted triangles depict the trimeric spike protein IgG levels. Circles with black borders illustrate naïve animals. Anti-spike GMT at day 28 from naïve and treated groups were determined by ELISA. Data are presented as mean±SEM of 10 animals per group. A one-way ANOVA with Tukey's multiple comparison test was performed to assess significance. P values≤0.0001 are represented with four asterisks. P values≤0.001 are represented with three asterisks.

FIG. 19 shows the results of cell-based (hACE2-HEK293T) surrogate neutralization assays on immunized mice after a single shot with FL-S eVLPs or the trimeric spike protein on day 20 at dilutions of 1:25 (unadjuvanted (Ag only), adjuvanted with Adju-Phos®, or naïve) for SARS-CoV-2 reference strain (Wuhan). Black circles represent FL-S eVLPs whereas inverted triangles depict the trimeric spike. Circles with black borders illustrate naïve animals. Data are presented as mean±SEM of 10 animals per group. A one-way ANOVA with Tukey's multiple comparison test was performed to assess significance. P values≤0.0001 and p≤0.01 are represented with four and two asterisks respectively.

FIG. 20 shows the results of cell-based (hACE2-HEK293T) surrogate neutralization assays on immunized mice after a single shot with FL-S eVLP or the trimeric spike protein on day 20 at dilutions of 1:75 (AS01b) for SARS-CoV-2 reference strain (Wuhan). Black circles represent FL-S eVLP whereas inverted triangles depict the trimeric spike. Circles with black borders illustrate naïve animals. Data are presented as mean±SEM of 10 animals per group. A one-way ANOVA with Tukey's multiple comparison test was performed to assess significance. P values ≤0.0001 are represented with four asterisks.

FIGS. 21A-D show that a terminal deletion of 18 amino acids from the SARS-CoV-2 S C-tail leads to an increase in virus-like particles in cell supernatants. FIG. 21A shows total protein staining of cushion-sedimented S-VLPs produced by S-HA, WT-S, (no HA tag) and SΔ18 expression. FIG. 21B shows total protein staining on purified S-VLPs produced by S-HA, WT-S (no HA tag) and SΔ18 expression. Each condition shows the downstream purification fractions: feed, flow-through and purified. FIG. 21C shows negative-stain TEM of purified S-VLP samples of S-HA, WT-S (no HA tag) and SΔ18. A 200 nm scale bar is shown at the bottom of each image. FIG. 21D shows a representative immunoblot of the downstream purification fractions (feed, flow-through and purified) of S-VLPs-HA or SΔ18 stained with anti-HA or anti-S(S2A4).

FIGS. 22A-C show generation of bivariant SARS-CoV-2 VLPs by the co-expression of S proteins of different virus strains. FIG. 22A shows total protein staining of VLP purification fractions produced by the expression of S-o-B.1.1.529-HA, S-o-BA4/BA5-HA, SΔ18/S-o-B.1.1.529-HA, SΔ18/S-o-BA4/BA5-HA in CHO-C2 cells. FIG. 22B shows representative immunoblot of affinity-purified samples using AVIPure-COV2S VLP resin. From left to right, -o-B.1.1.529-HA, S-o-BA4/BA5-HA, SΔ18/S-o-B.1.1.529-HA, SΔ18/S-o-BA4/BA5-HA. The top blot shows anti-HA staining whereas the bottom blot is stained with anti-Spike S2A4. FIG. 22C shows negative-stain TEM of purified bivariant S-VLP samples of SΔ18/S-o-B.1.1.529-HA and SΔ18/S-o-BA4/BA5-HA. A 200 nm scale bar is shown at the bottom of each image.

FIGS. 23A-D show that S-VLPs displaying Influenza H1 antigens are formed and purified from CHO-C2 cells co-expressing S-HA and H1. FIG. 23A shows a representative immunoblot of cell extracts, supernatants and cushion-sedimented samples produced by expression of S-HA, H1 and S-HA/H1. The top blot shows anti-Spike (S1) staining whereas the bottom blot is stained with anti-H1. FIG. 23B shows total protein staining and immunoblot of affinity-purified (AVIPure-COV2S VLP resin) samples S-HA, H1 and S-HA/H1. On the right side, the top blot shows anti-S1 staining whereas the bottom one is stained with anti-H1. FIG. 23C shows negative-stain TEM of purified S-VLP, H1 and bivalent S-HA/H1 VLPs. A 100 or 400 nm scale bar is shown at the bottom of each image. FIG. 23D shows immunogold labeling on S-HA/H1-VLPs. S-HA and H1 antigens were immunolabeled with VHH-72-hFc anti-Spike and anti-H1 followed by gold-conjugated secondary antibodies of 18 nm (black arrows), or 10 nm (white arrows) respectively. A negative-stain TEM micrograph (without antibody labelling) is shown at the bottom right. A 50, 100, or 200 nm scale bar is shown at the bottom of each image.

FIG. 24 shows that S-VLPs can incorporate two Influenza antigens: hemagglutinin (H1) and neuraminidase (N1). A representative immunoblot of cell extracts, supernatants (concentrated) and purified samples produced by the expression of S-HA, H1, N1 and S-HA/H1/N1 in CHO-C2 cells is shown. From top to bottom: anti-Spike (S1), anti-H1 and anti-N1.

FIGS. 25A-C show bivalent S-VLPs displaying F protein from RSV. FIG. 25A shows a representative immunoblot of cell extracts, supernatants (concentrated) and cushion-sedimented samples produced by the expression of S-HA, F and S-HA/RSVF in CHO-C2 cells. From top to bottom: anti-Spike (S1), anti-F. FIG. 25B shows a representative immunoblot of cell extracts, supernatants (concentrated) and purified samples produced by the expression of F and SΔ18/RSVF in CHO-C2 cells. From top to bottom: anti-Spike (S1), anti-F. FIG. 25C shows immunogold labeling on SΔ18/RSVF-VLPs. S and F antigens were immunolabeled with VHH-72-hFc anti-Spike and anti-F followed by gold-conjugated secondary antibodies of 18 nm (black arrows), or 10 nm (white arrows) respectively. A 200 nm scale bar is shown at the bottom of the image.

FIGS. 26A-C show that S-VLPs can harbor human membrane proteins when overexpressed with SΔ18 in CHO-C2 cells. FIG. 26A shows a representative immunoblot of cell extracts and purified S-VLPs produced by the co-expression of SΔ18 with human muscarinic acetylcholine receptor M4 or GALR2. From top to bottom: anti-Spike (S1) and anti-HA (M4 and GALR2 are HA-tagged products at C-terminus). FIGS. 26B and C show immunogold labeling on S/M4 (FIG. 26B) and S/GALR2 (FIG. 26C) VLPs. S and Flag antigens (M4 and GALR2 are Flag-tagged products at N-terminus) were immunolabeled with VHH-72-hFc anti-Spike and anti-Flag followed by gold-conjugated secondary antibodies of 18 nm (black arrows), or 10 nm (white arrows) respectively. A 100 and 200 nm scale bar is shown at the bottom of the image.

FIGS. 27A to 27D show representative lanes of SDS-PAGE gels containing monovariant, bivariant, bivalent or trivalent eVLPs and densitometry plots performed thereon. On the left of each image is shown an SDS-PAGE lane with a specific eVLP sample. On the right, is shown a densitometric plot for the eVLP sample. The x-axis shows the relative front (distance from the top of the lane to the band) whereas the y-axis displays intensity units. FIG. 27A shows results for a monovariant eVLP (FL-S-HA, Wuhan), FIG. 27B shows results for a representative purified bivariant eVLP (SΔ18/S-omicron-B.1.1.529-HA), FIG. 27C shows results for a representative purified bivalent eVLP (FL-S-HA/H1), and FIG. 27D shows results for a representative purified trivalent eVLP (FL-S-HA/H1/N1).

### DETAILED DESCRIPTION

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures, published sequences, and other references mentioned herein are expressly incorporated by reference in their entirety.

### Definitions

As used herein, the following terms may have meanings ascribed to them below, unless specified otherwise. However, it should be understood that other meanings that are known or understood by those having ordinary skill in the art are also possible, and within the scope of the present disclosure. In the case of conflict, the present specification, including definitions, will control. In addition, the materials, methods, and examples are illustrative only and not intended to be limiting.

The term “about” as used herein may be used to take into account experimental error, measurement error, and variations that would be expected by a person having ordinary skill in the art. For example, “about” may mean plus or minus 10%, or plus or minus 5%, of the indicated value to which reference is being made.

As used herein the singular forms “a”, “an”, and “the” include plural references unless the context clearly dictates otherwise. For example, a composition defined as comprising “a” (or “an”, or “the”) given component must comprise at least one of the given component, but it may also comprise a plurality of the given component. Similarly, a method of producing “a” (or “an”, or “the”) product must produce at least one of the product, but it may also produce a plurality of the product.

The phrase “and/or”, as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

The term “sequence identity” as used herein refers to the percentage of sequence identity between two amino acid sequences or two nucleotide sequences. To determine the percent identity of two amino acid sequences or of two nucleotide sequences, the sequences are aligned for optimal comparison purposes (e.g., gaps can be introduced in the sequence of a first amino acid or nucleotide sequence for optimal alignment with a second amino acid or nucleotide sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity-number of identical overlapping positions/total number of positions.times.100%). In one embodiment, the two sequences are the same length. The determination of percent identity between two sequences can also be accomplished using a mathematical algorithm. One non-limiting example of a mathematical algorithm utilized for the comparison of two sequences is the algorithm of Karlin and Altschul, 1990, modified as in Karlin and Altschul, 1993. Such an algorithm is incorporated into the NBLAST and XBLAST programs of Altschul et al., 1990. BLAST nucleotide searches can be performed with the NBLAST nucleotide program parameters set, e.g., for score=100, wordlength=12 to obtain nucleotide sequences homologous to a nucleic acid molecule of the present disclosure. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997. Alternatively, PSI-BLAST can be used to perform an iterated search which detects distant relationships between molecules. When utilizing BLAST, Gapped BLAST, and PSI-Blast programs, the default parameters of the respective programs (e.g., of XBLAST and NBLAST) can be used (see, e.g., the NCBI website). Another non-limiting example of a mathematical algorithm utilized for the comparison of sequences is the algorithm of Myers and Miller, 1988. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, typically only exact matches are counted.

As used herein, the term “codon-optimized” is used to indicate that the sequence of a nucleic acid molecule has been altered, relative to that of a reference sequence (e.g., a naturally-occurring sequence or a sequence that is known to the public, for example by publication in the scientific literature or in an online database, such as GenBank), to introduce silent mutations (also referred to as synonymous mutations or synonymous substitutions) to change the nucleotide sequence of the nucleic acid molecule, without altering the amino acid sequence encoded by the nucleic acid molecule, to reflect the codon bias of the host cell or organism in which the nucleic acid molecule is to be expressed, for the purpose of improving gene expression and translational efficiency in the host cell or organism. Codon-optimization is well established in the art of molecular biology and codon-optimization tools (e.g., algorithms) are readily available. By comparing the nucleotide sequence of a nucleic acid molecule to a reference sequence, a person of skill in the art will recognize when a nucleic acid molecule is codon-optimized.

As used herein, the term “conservative mutation” or “conservative amino acid substitution” refers to the substitution of an amino acid with another amino acid having similar physical or chemical characteristics. For example, a conservative amino acid substitution may substitute a basic, neutral, hydrophobic, or acidic amino acid for another of the same group. By the term “basic amino acid” it is meant hydrophilic amino acids having a side chain pKa value of greater than 7, which are typically positively charged at physiological pH. Basic amino acids include arginine (Arg or R) and lysine (Lys or K). By the term “neutral amino acid” (also “polar amino acid”), it is meant hydrophilic amino acids having a side chain that is uncharged at physiological pH, but which has at least one bond in which the pair of electrons shared in common by two atoms is held more closely by one of the atoms. Polar amino acids include serine (Ser or S), threonine (Thr or T), cysteine (Cys or C), tyrosine (Tyr or Y), asparagine (Asn or N), and glutamine (Gln or Q). The term “hydrophobic amino acid” (also “non-polar amino acid”) is meant to include amino acids exhibiting a hydrophobicity of greater than zero according to the normalized consensus hydrophobicity scale of (Eisenberg et al, 1984). Hydrophobic amino acids include proline (Pro or P), isoleucine (Ile or I), phenylalanine (Phe or F), valine (Val or V), leucine (Leu or L), tryptophan (Trp or W), methionine (Met or M), alanine (Ala or A), and glycine (Gly or G). “Acidic amino acid” refers to hydrophilic amino acids having a side chain pKa value of less than 7, which are typically negatively charged at physiological pH. Acidic amino acids include glutamate (Glu or E), and aspartate (Asp or D). Histidine (His or H) is a polar amino acid with a special ionization potential due to its pKa around 7, and more precisely around 6.4 in case of histidine residues located at the protein surface (Tanokura, 1983). This results in histidine amino acid residues being “polar” and predominantly uncharged at physiological pH of 7.2-7.4, and predominantly positively charged in acidic environments (pH<7).

A conservative amino acid substitution may be selected from the substitutions set forth in Table 1.

TABLE 1
Conservative amino acid substitutions

| Amino Acid | Conservative Substitutions |
|---|---|
| Ala (A) | Pro, Gly, Thr |
| Arg (R) | His, Lys, Glu, Gln |
| Asn (N) | Asp, Ser, Gln |
| Asp (D) | Asn, Glu, Gln |
| Cys (C) | Ser, Thr, Met |
| Gln (Q) | Glu, His, Lys, Asn, Thr, Arg |
| Glu (E) | Gln, Asp, Lys, Asn, His, Arg |
| Gly (G) | Ala, Thr, Pro, Ser |
| His (H) | Gln, Arg, Lys, Glu, Thr |
| Ile (I) | Met, Leu, Phe, Val, Tyr |
| Leu (L) | Ile, Met, Phe, Val, Tyr |
| Lys (K) | Arg, Glu, Gln, His |
| Met (M) | Ile, Leu, Phe, Val |
| Phe (F) | Met, Tyr, Ile, Leu, Trp, Val |
| Pro (P) | Ala, Thr, Gly |
| Ser (S) | Thr, Gly, Asn |
| Thr (T) | Pro, Ser, Ala, Gly, His, Gln |
| Tyr (Y) | Phe, Trp, Met, Ile, Val, Leu |
| Val (V) | Met, Ile, Tyr, Phe, Leu |

As used herein the terms “peptide”, “polypeptide”, and “protein” refer to a linear chain of two or more amino acids joined by peptide bonds. The term “peptide” is generally used to refer to a short chain of amino acids comprising 2 to 49 amino acids, whereas the term “polypeptide” is generally used to refer to a longer chain of amino acids comprising 50 or more amino acids. The term “protein” is generally used to refer to one or more peptides or polypeptides that have been folded and/or assembled to form a three-dimensional structure. However, the terms “peptide”, “polypeptide”, and “protein” may be used interchangeably. Further, a protein may include post-translational modifications, as will be understood to one skilled in the art. Examples of post-translational modification include, but are not limited to, glycosylation, lipidation, phosphorylation, ubiquitination, acetylation, nitrosylation, and methylation.

As used herein, the term “recombinant protein” refers to a protein that is produced by recombinant techniques, wherein generally DNA or RNA encoding the expressed protein is inserted into a suitable expression vector that is in turn introduced into a host cell to allow expression of the recombinant protein. Recombinant proteins may include amino acid sequences from two or more sources, such as different proteins. Such recombinant polypeptides may be referred to as fusion polypeptides, fusion proteins, or fusion constructs. Recombinant proteins may also include one or more synthetic amino acid sequences.

As used herein, the term “pharmaceutically acceptable carrier” refers to a carrier that is non-toxic. Suitable pharmaceutically acceptable carriers include, for example, one or more of water, saline, phosphate buffered saline, dextrose, glycerol, ethanol, and combinations thereof. Pharmaceutically acceptable carriers may further contain minor amounts of auxiliary substances such as wetting or emulsifying agents, preservatives, or buffering agents that enhance shelf life or effectiveness.

As used herein, the terms “prevention”, “preventing”, “prevent”, and grammatical variations thereof are used herein to refer to actions, steps, or measures that are taken in an effort to protect a subject from infection or disease. Prevention may or may not provide complete protection. For example, the subject may still become infected and/or exhibit symptoms of the disease, though perhaps with milder (or no) symptoms. The degree of protection resulting from prevention may vary across a population, with some members of the population exhibiting a high degree of protection against the infection or disease, while other members of the population exhibit little to no protection against the infection or disease.

As used herein, the term “subject” is used to refer to an animal, including a human or other mammal, that is susceptible to infection by SARS-CoV-2.

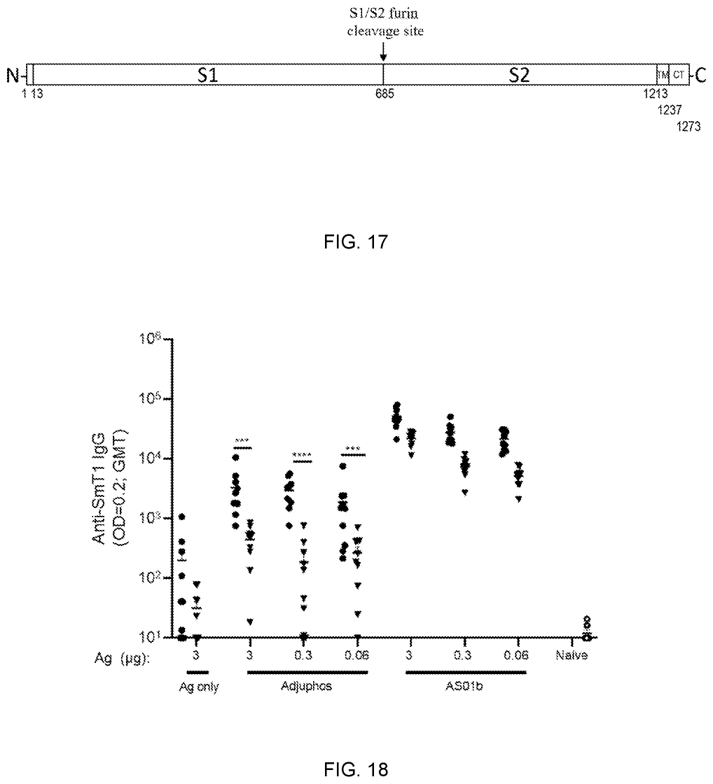

As used herein, the term “SARS-CoV-2 spike protein”, which may be abbreviated as “S”, refers to a spike protein of a SARS-CoV-2 virus. Full-length spike protein comprises an S1 domain comprising N-terminal domain (NTD) and a receptor binding domain (RBD), and an S2 domain comprising a fusion peptide (FP), an internal fusion peptide (IFP), heptad repeats (HR), a transmembrane domain(TM) and a C-terminal tail (CT). The spike protein also includes a signal peptide that is cleaved to form mature spike protein. SARS-CoV-2 entry into a host cell requires sequential cleavage of the spike protein at S1/S2 and S2′ cleavage sites to mediate membrane fusion. A furin recognition site (RRAR) is present at the S1/S2 cleavage site allowing cleavage by a host enzyme (e.g., a furin-like or other protease). The general structure of the SARS-CoV-2 spike protein is shown in FIG. 17 and described in greater detail in Wrapp et al (2020a), Liangwei et al (2020) and Xia (2021). A reference sequence for SARS-CoV-2 spike protein is GenBank accession no. YP_009724390.1 (SEQ ID NO: 2). The amino acid sequence set forth in SEQ ID NO: 2 includes the signal peptide (amino acid residues 1 to 13), which is not present in the mature protein. The term “SARS-CoV-2 spike protein” is not intended to be limited to a SARS-CoV-2 protein comprising the specific sequence set forth in SEQ ID NO: 2 (either with or without the signal peptide). Rather, this term is intended to include any and all variants of the SARS-CoV-2 spike protein that may be found in a SARS-CoV-2 viral strain. This term is further intended to include artificial SARS-CoV-2 spike proteins that have been engineered to include desired features, such as linkers, tags, fusions to other polypeptides, or desired mutations, including amino acid substitutions, deletions, and/or insertions. Desired mutations may, for example, alter the stability or conformation (e.g., pre-fusion or RBD-up/down conformations) of the spike protein, or introduce or remove an enzyme recognition site in the spike protein. Further, the nucleic acid molecule encoding the SARS-CoV-2 spike protein may be engineered to include desired features, such as endonuclease recognition sites to facilitate cloning or genetic engineering, that result in minor changes to the SARS-CoV-2 spike protein amino acid sequence. For example, SEQ ID NOs: 15, 17, 19, 21, 23, and 25 include an added KpnI recognition site, added for the purpose of facilitating cloning, that results in a two amino acid insertion (Cys Thr) in the SARS-CoV-2 spike protein sequence. This CT insertion is present in SEQ ID NOs: 16, 18, 20, 22, 24, and 26, but its presence is believed not to be required for FL-S eVLP formation. The term “SARS-CoV-2 spike protein” is also intended to include a SARS-CoV-2 spike protein having an N- and/or C-terminal truncation, in which a number of N-terminal and/or C-terminal amino acids are omitted from the protein. Typically, such truncations will include a relatively small number of amino acids (for example 1, 2, 3, 4, 5, 6, 7, 8, 9, or 10 amino acids omitted from the N- and/or C-terminus), although larger truncations are also envisioned and may be encompassed by the term “SARS-CoV-2 spike protein”. Preferably alterations in the SARS-CoV-2 spike protein do not abolish its folding or assembly. In a particular embodiment, the SARS-CoV-2 spike protein has at least 70%, at least 75%, at least 80%, at least 85%, at least 90%, at least 91%, at least 92%, at least 93%, at least 98%, at least 94%, at least 95%, at least 96%, at least 97%, at least 98%, at least 99%, or 100% sequence identity to the full length of the amino acid sequence set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, or SEQ ID NO: 24.

The SARS-CoV-2 spike protein may comprise one or more mutations to stabilize it in the prefusion conformation. During SARS-CoV-2 entry, for membrane fusion to take place, the SARS-CoV-2 spike protein must undergo a structural transition from the prefusion conformation to the postfusion conformation. The prefusion dynamics of the spike trimer alternate between three receptor binding domains (RBD) in close position (RBD-down) to one open conformation (RBS-up). An RBD-up monomer is sufficient to bind the angiotensin converting enzyme 2 (ACE2) receptor on the host cell surface. ACE2 then exposes the S2′ cleavage site of the spike protein which is catalyzed by the host serine protease TMPRSS2. This reaction sheds the S1 domains in the trimer exposing the fusion peptides (postfusion conformation), triggering downstream events to fuse the viral envelope and the cell membrane for virus entry (Jackson C et al, 2022). The prefusion conformation of the spike trimer is thought to be a suitable therapeutic target, however this conformation is naturally transient and unstable. To overcome this, various mutations have been introduced into the spike protein to stabilize the trimer in the prefusion conformation. Suitable mutations will be known to one skilled in the art, for example as described in Wrapp et al (2020b), Hsieh et al (2020), Schaub et al (2021), Riley et al (2021), and Juraszek et al (2021). One example of such a mutation is the 2P mutation described in Wrapp et al (2020b), in which two stabilizing proline mutations (K986P, V987P) are introduced in the C-terminal S2 fusion machinery. This mutation has proved effective for a variety of betacoronavirus S proteins. Another example of such a mutation is the HexaPro mutation (F817P, A892P, A899P, A942P, K986P, V987P) described in Hsieh et al (2020). Numerous other examples exist, as will be known to one skilled in the art.

Naturally-occurring SARS-CoV-2 spike protein comprises a furin cleavage site at the S1/S2 junction, having the sequence PRRAR at positions 681-685 relative to the reference sequence GenBank Accession No. YP_009724390.1 (SEQ ID NO: 2). Herein, a SARS-CoV-2 spike protein may be referred to as having an “inactivated” S1/S2 furin cleavage site if the spike protein comprises one or more mutations that prevent cleavage by furin at the S1/S2 junction. One example of an inactivating mutation is a modification of the S1/S2 furin cleavage site from PRRAR to PGGAS, however other inactivating mutations are possible and any SARS-CoV-2 spike protein comprising one or more mutations that substantially reduce or eliminate cleavage by furin at the S1/S2 junction may be considered to be a spike protein comprising an inactivated S1/S2 furin cleavage site.

The SARS-CoV-2 spike protein comprises an endoplasmic reticulum (ER) retention signal (KLHYT, SEQ ID NO: 48) at its C-terminus. The phrase “mutation that reduces or eliminates the function of the ER retention signal” refers to a mutation that alters this sequence and reduces or eliminates ER retention of the spike protein. The mutation may, for example, be a substitution, rearrangement, and/or deletion of one or more amino acids within the ER retention signal; a deletion that removes a portion of the ER retention signal; or a deletion that removes the entire ER retention signal. In a particular embodiment, the mutation is a terminal deletion of 5 to 21 contiguous amino acids from the C-terminus of the spike protein.

As used herein, “substantially full-length recombinant SARS-CoV-2 spike protein” (abbreviated as FL-S) refers to a spike protein of a SARS-CoV-2 virus, including both naturally-occurring variants of the spike protein that may be found in a SARS-CoV-2 virus and genetically modified or engineered artificial variants of the spike protein, produced by recombinant techniques, that includes at least the S1 subunit, the S2 subunit (including the transmembrane domain or a functional fragment thereof, and optionally including the C-terminal tail or a fragment thereof) of the SARS-CoV-2 spike protein (see FIG. 17 for a schematic representation of the SARS-CoV-2 spike polypeptide). “FL-S-HA” refers to an HA-tagged version of the FL-S protein that includes a hemagglutinin (HA) tag at its C-terminus. In the examples described herein, an HA tag was included to aid protein purification and detection, but the presence of the HA tag is optional and it is not required for FL-S eVLP formation. A “substantially full-length recombinant SARS-CoV-2 spike protein” may be full-length, i.e., it may include the complete amino acid sequence of a SARS-CoV-2 spike protein as may be found in a SARS-CoV-2 virus, either before or after cleavage of the signal peptide, and optionally including one or more amino acid insertions and/or substitutions but no amino acid deletions relative to the sequence of the spike protein of the SARS-CoV-2 virus. A “substantially full-length recombinant SARS-CoV-2 spike protein” may also include one or more small internal deletions (e.g., up to about 1, up to about 2, up to about 3, up to about 4, up to about 5, up to about 6, up to about 7, or up to about 8 amino acids in length per deletion) and/or small N-terminal and/or C-terminal truncations (e.g., up to about 5, up to about 10, up to about 15, up to about 20, up to about 25, up to about 30, up to about 35, or up to about 40 amino acids in length) relative to a naturally-occurring SARS-CoV-2 spike polypeptide sequence and still be considered to be substantially full-length, provided it retains the ability to able to fold and trimerize, and provided that the transmembrane domain is still functional to allow insertion into an animal cell membrane. A “substantially full-length recombinant SARS-CoV-2 spike protein” may also include one or more mutations (e.g., amino acid insertions and/or substitutions) relative to a naturally-occurring SARS-CoV-2 spike protein, provided it retains the ability to able to fold and trimerize, and provided that the transmembrane domain is still functional to allow insertion into an animal cell membrane. In a specific embodiment, the “substantially full-length recombinant SARS-CoV-2 spike protein” is about 1220 to about 1280, or about 1230 to about 1270, or about 1240 to about 1270, or about 1250 to about 1270, or about 1255 to about 1265, or about 1260 amino acids in length, after cleavage of the signal peptide and not including in the length calculation any heterologous amino acid sequences (such as affinity tags or other non-spike amino acid sequences) that may be fused to the SARS-CoV-2 spike protein, typically at the N- and/or C-terminus.

As used herein, the term “multivariant”, when used in reference to an eVLP, refers to an eVLP that comprises two or more variants of a substantially full-length recombinant SARS-CoV-2 spike protein. An eVLP comprising two spike variants of a substantially full-length recombinant SARS-CoV-2 spike protein may be referred to as a “bivariant” eVLP.

As used herein, the term “multivalent”, when used in reference to an eVLP, refers to an eVLP that comprises at least one substantially full-length recombinant SARS-CoV-2 spike protein variant and at least one additional recombinant protein other than a substantially full-length recombinant SARS-CoV-2 spike protein. An eVLP comprising only one substantially full-length recombinant SARS-CoV-2 spike protein variant and only one additional recombinant protein may also be referred to as a “bivalent” eVLP. In a particular embodiment, the at least one additional recombinant protein is a membrane-associated protein. The at least one additional recombinant protein may, for example, be a cell membrane protein or a viral antigen.

An eVLP as described herein may be both multivariant and multivalent (i.e., an eVLP could comprise two or more substantially full-length recombinant SARS-CoV-2 spike protein variants and at least one additional recombinant protein other than a substantially full-length recombinant SARS-CoV-2 spike protein).

As used herein, the term “SARS-CoV-2 envelope protein”, which may be abbreviated as “E”, refers to an envelope protein of a SARS-CoV-2 virus. A reference sequence for a SARS-CoV-2 envelope protein is GenBank accession no. YP_009724392.1, however this term is intended to include any and all variants of the SARS-CoV-2 envelope protein that may be found in a SARS-CoV-2 viral strain, as well as functional variants of the SARS-CoV-2 envelope protein that have been created through genetic engineering or recombinant DNA technology.

As used herein, the term “SARS-CoV-2 membrane protein”, which may be abbreviated as “M”, refers to a membrane protein of a SARS-CoV-2 virus. A reference sequence for a SARS-CoV-2 membrane protein is GenBank accession no. YP_009724393.1, however this term is intended to include any and all variants of the SARS-CoV-2 membrane protein that may be found in a SARS-CoV-2 viral strain, as well as functional variants of the SARS-CoV-2 membrane protein that have been created through genetic engineering or recombinant DNA technology.

As used herein, the term “SARS-CoV-2 nucleocapsid protein”, which may be abbreviated as “N”, refers to a nucleocapsid protein of a SARS-CoV-2 virus. A reference sequence for a SARS-CoV-2 membrane protein is GenBank accession no. YP_009724397.2, however this term is intended to include any and all variants of the SARS-CoV-2 nucleocapsid protein that may be found in a SARS-CoV-2 viral strain, as well as functional variants of the SARS-CoV-2 nucleocapsid protein that have been created through genetic engineering or recombinant DNA technology.

As used herein, the term “heterologous polypeptide” refers to a polypeptide derived from a different source than the reference polypeptide. For example, a SARS-CoV-2 spike protein may be fused to a heterologous polypeptide that is derived from a source other than SARS-CoV-2 S. The heterologous polypeptide may be any polypeptide that is compatible with incorporation of the SARS-CoV-2 spike protein into an eVLP. In a particular embodiment, the heterologous polypeptide may be an epitope tag, such as a FLAG tag, a poly-histidine tag, a c-Myc tag, an HA tag, glutathione S-transferase (GST), maltose binding protein (MBP), green fluorescent protein (GFP), red fluorescent protein (RFP), or mCherry.

As used herein, the term “animal host cell” refers to a cell of an animal, preferably an animal cell that is grown in culture. Commonly used animal cell lines include, for example, insect cells, such as Spodoptera frugiperda cells (e.g., Sf9 or Sf21), Trichoplusia ni cells (e.g., High Five cell lines), and Drosophila melanogaster cells (e.g., Schneider S2); and mammalian cells, such as human embryonic kidney cells (e.g., HEK293) and Chinese hamster ovary (CHO) cells. In a particular embodiment, the animal host cell is a mammalian host cell. In a specific embodiment, the mammalian host cell is a CHO cell.

As used herein, the term “expression vector” refers to a vector into which a nucleic acid molecule may be introduced to allow the nucleic acid molecule to be expressed (transcribed) by a host cell. The expression vector may comprise regulatory elements that allow for expression of the nucleic acid molecule within a particular host cell. Such regulatory elements may allow for constitutive, conditional, or cell-type-specific expression. As will be apparent to one skilled in the art, a suitable expression vector may be selected based on the host cell type and what type (e.g., constitutive vs. conditional) and/or level of expression is desired. Commonly employed vectors include plasmids and viral vectors.

As used herein, the term “enveloped virus-like particle” (eVLP) refers to a virus-like particle (VLP) comprising an envelope acquired from a host cell with one or more viral proteins present on a membrane surface of the eVLP (i.e., at least a portion of the viral protein is exposed on the inner and/or outer surface of the membrane envelope). More specifically, this term is used to refer to a self-assembled enveloped virus-like particle comprising a SARS-CoV-2 spike protein ectodomain displayed on its outer surface that is produced by expressing a substantially full-length recombinant SARS-CoV-2 spike protein (FL-S) in a mammalian host cell, such as a CHO cell, thereby inducing the cell to form eVLPs comprising the SARS-CoV-2 spike protein. These eVLPs are released by the cell into the surrounding medium (e.g., a cell culture medium or other medium suitable to allow expression of the FL-S by the animal cell) from which the eVLPs can be purified.

As used herein, a “recombinant viral protein substantially capable of independently inducing eVLP formation by the host cell” refers to a recombinant viral protein that, if expressed in the animal host cell on its own, is sufficient to induce the spontaneous release by the cell of significant numbers of eVLPs comprising the viral protein, without need for co-expression or provision of any other recombinant protein. Examples of proteins that are known to be substantially capable of independently inducing animal cells to form eVLPs include retroviral Gag proteins, influenza matrix protein, SARS-CoV-2 envelope and membrane proteins (expressed together), hepatitis B surface antigen, and Ebola virus VP40.

As used herein, the “total protein content” of an eVLP refers to the total amount of protein, by weight, present in the eVLP. The amount of a particular protein in an eVLP may be expressed as a percentage of the total protein content. Methods to determine the percentage of total protein content are known and include methods such as chromatography or densitometry analysis performed on a stained SDS-PAGE gel of total protein.

As used herein, the terms “isolated”, “isolation”, “purified”, “purification”, “purify” and like terms when used in relation to an eVLP derived from a cell, refer to enrichment of the eVLP relative to other components normally present in the cell and/or in the medium in which the cell is cultured that do not form part of the eVLP. The term “purified” does not necessarily indicate that complete purity has been achieved, merely that a significant portion of other components has been removed. For example, removal of 75% or more of the other components may be sufficient to consider a eVLP as having been purified, though the level of purity may be higher. For example, 80% or more, 85% or more 90% or more, or 95% or more of other components may be removed during the purification of an eVLP. Similarly, the term “isolated” does not necessarily mean that the eVLP has been completely isolated from all other components. Further, an “isolated” eVLP need not be in isolation. It may be included in a composition with one or more additional components, such as a buffer, solution, adjuvant, preservative, etc.

As used herein, the terms “co-express”, “co-expression”, and grammatical variations thereof refer to expression of two or more gene products (e.g., proteins or RNAs) by a host cell, such that the two or more gene products are present in the host cell at the same time. Expression of the two or more gene products may be simultaneous, sequential, or overlapping; as long as the two or more gene products resulting from the expression are present in the cell simultaneously for at least a period of time.

As used herein, the term “endogenous retrovirus sequence” refers to an endogenous-retrovirus like sequence that is naturally present within the genome of a CHO cell. A “functional endogenous retrovirus sequence” is one that is transcribed and translated by the CHO cell, contributing to the generation of retroviral-like particles by the CHO cell. Examples of functional endogenous retrovirus sequences are sequences with open reading frame(s) that encode functional Gag, Pol and/or Env protein(s).

It should also be understood that, in certain methods described herein that include more than one step or act, the order of the steps or acts of the method is not necessarily limited to the order in which the steps or acts of the method are recited unless the context indicates otherwise.

### Details

The present inventors have surprisingly found that enveloped virus-like particles (eVLPs) can be produced by expressing a substantially full-length recombinant SARS-CoV-2 spike protein (FL-S) in an animal host cell, such as but not limited to a mammalian host cell such as a Chinese hamster ovary (CHO) cell, without need for co-expression of SARS-CoV-2 envelope (E), membrane (M), and/or nucleocapsid (N) protein, or any other recombinant protein. When FL-S is expressed in the host cell, the host cell spontaneously produces and releases eVLPs comprising FL-S into a surrounding medium, from which the eVLPs can be isolated or purified. In addition to comprising FL-S, the eVLP is expected to comprise one or more membrane components from the host cell. The FL-S may comprise one or more FL-S variants and it may further comprise one or more additional recombinant proteins, other than FL-S.

CHO cells contain type-C endogenous retrovirus sequences in their genome and they are known to release retroviral-like particles. To aid in purification of eVLPs from CHO cells, a CHO cell may be used that comprises one or more mutations that inactivate one or more functional endogenous retrovirus sequences in its genome. Inactivation of one or more endogenous retrovirus sequence(s) is expected to reduce the number of extracellular retrovirus-like particles produced by the CHO cell. Disruption of retrovirus sequences in CHO cells has been described in the art (e.g., see Duroy et al, 2020) and a person of skill in the art would be familiar with methods that could be used to disrupt one or more endogenous retrovirus sequences in a CHO cell.

The FL-S should comprise at least the S1 subunit, the S2 subunit, and the transmembrane domain of a SARS-CoV-2 spike protein. FL-S may further comprise the cytoplasmic tail. The S1/S2 furin cleavage site of the spike protein may be inactivated to prevent cleavage between the S1 and S2 subunits, however low levels of cleavage may still occur. When a spike protein sequence is provided herein, it is intended to represent the sequence that is present in the mature spike protein. The amino acid sequence may or may not be present as a single contiguous polypeptide. For example, the S1 and S2 subunits may be separated by cleavage.

The FL-S preprotein may comprise a signal peptide. The signal peptide may be an endogenous SARS-CoV-2 spike protein signal peptide or it may be a heterologous or synthetic signal peptide. Due to cleavage, the signal peptide is not expected to be present in the mature FL-S protein found in the eVLPs. Because of this, the signal peptide sequence is not included in SEQ ID NOs: 2, 4, 5, 7, 8, 10, 11, 13, 14, 16, 18, 20, 22, 24, 44, 45, 46, or 47.

The FL-S may comprise an amino acid sequence having at least 70%, at least 75%, at least 80%, at least 85%, at least 90%, at least 95%, at least 96%, at least 97%, at least 98%, or at least 99% sequence identity to, or it may comprise or consist of, the full length of the amino acid sequence set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47. In an embodiment, the eVLP is a multivariant eVLP comprising two or more FL-S protein variants. In a further embodiment, each FL-S may comprise an amino acid sequence as defined in this paragraph.

The FL-S may comprise an amino acid sequence as set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47, except that the amino acid sequence comprises between one and 60 amino acid substitutions, insertions, and/or deletions relative to the provided sequence. For example, the FL-S may comprise 1, up to 5, up to 10, up to 15, up to 20, up to 25, up to 30, up to 35, up to 40, up to 45, up to 50, up to 55, or up to 60 amino acid substitutions, insertions, and/or deletions relative to the provided sequence. In an embodiment, the eVLP is a multivariant eVLP comprising two or more FL-S variants. In a further embodiment, each FL-S variant may comprise an amino acid sequence as defined in this paragraph.

The FL-S may comprise an amino acid sequence as set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47, except that the amino acid sequence comprises between one and 20 amino acid substitutions relative to the provided sequence. For example, the FL-S may comprise 1, 2, 3, 4, 5, 6, 7, 8, 9, 10, 11, 12, 13, 14, 15, 16, 17, 18, 19 or 20 amino acid substitutions relative to the provided sequence. The amino acid substitutions may be conservative, non-conservative, or a combination of both conservative and non-conservative substitutions. In a specific embodiment, all of the amino acid substitutions are conservative substitutions. In an embodiment, the eVLP is a multivariant eVLP comprising two or more FL-S variants. In a further embodiment, each FL-S variant may comprise an amino acid sequence as defined in this paragraph. In some embodiments, the FL-S, or in the case of a multivariant eVLP the FL-S variants together, may make up at least 50%, at least 55%, at least 60%, at least 65%, at least 70%, at least 75%, or at least 80% of the total protein content of the eVLP.

In an embodiment, the FL-S, or each FL-S variant, is stabilized in the prefusion conformation. Various mutations for stabilizing a SARS-CoV-2 spike protein in the prefusion conformation are known in the art, including the 2P mutation (Wrapp et al, (2020b) and the HexaPro mutation (Hsieh et al, 2020), among others.

The animal cell may produce a plurality of eVLPs of varying sizes. In a specific embodiment, the eVLPs have an average diameter of about 130 nm to about 150 nm when first produced (i.e., on day 0). In another embodiment, the eVLPs may have an average diameter of about 100 nm to about 160 nm. In another embodiment, the eVLPs have a median diameter of about 115 nm to about 135 nm. In a particular embodiment, the cells may produce at least 1.0×10¹² or at least 1.0×10¹³ eVLPs per liter of culture.

In a particular embodiment, the host cell does not co-express together with the substantially full-length recombinant SARS-CoV-2 spike protein one or more of: SARS-CoV-2 envelope protein, SARS-CoV-2 membrane protein, and SARS-CoV-2 nucleocapsid protein. In an embodiment, the host cell does not co-express together with the substantially full-length recombinant SARS-CoV-2 spike protein SARS-CoV-2 envelope protein and/or SARS-CoV-2 membrane protein. In another embodiment the host cell co-expresses none of SARS-CoV-2 envelope protein, SARS-CoV-2 membrane protein, and SARS-CoV-2 nucleocapsid protein. In another embodiment, the host cell co-expresses neither SARS-CoV-2 envelope protein nor SARS-CoV-2 membrane protein. In another embodiment, FL-S is the only viral protein expressed by the host cell. In other embodiments, proteins from viruses other than SARS-CoV-2 may be co-expressed with FL-S if there is a desire for such proteins to be present in a multivalent eVLP together with FL-S. However, the co-expression of other viral proteins is not required for FL-S eVLP formation; expression of FL-S is sufficient.

In another embodiment, the host cell further expresses at least one additional recombinant protein other than FL-S and produces a multivalent eVLP comprising FL-S (which may be in either monovariant or multivariant form) and the at least one additional recombinant protein. The at least one additional recombinant protein may comprise any protein for which incorporation into an eVLP is desired. In an embodiment, the at least one additional recombinant protein comprises a viral antigen from a virus other than SARS-CoV-2. In an embodiment, the at least one additional recombinant protein comprises an influenza A antigen. In a particular embodiment, the at least one additional recombinant protein comprises influenza hemagglutinin and/or neuraminidase. In another particular embodiment, the additional recombinant protein comprises respiratory syncytial virus (RSV) fusion (F) glycoprotein.

The additional recombinant protein may also comprise a non-viral recombinant protein. In an embodiment, the at least one additional recombinant protein comprises a cell surface protein. In embodiments, the additional recombinant protein is a membrane protein. In particular embodiments, the additional recombinant protein may comprise human muscarinic acetylcholine receptor M4 or GALR2. In some embodiments, the FL-S may make up to at least 20%, at least 25%, at least 30%, at least 35%, at least 40%, at least 45%, at least 50%, at least 55%, or at least 60% of the total protein content of the multivalent eVLP. The nucleic acid molecule(s) encoding the at least one additional recombinant protein may be the same as, or separate from, the nucleic acid molecule(s) encoding FL-S.

The nucleic acid molecule used to express FL-S, or each FL-S variant, may comprise a nucleotide sequence encoding FL-S operatively linked to one or more than one heterologous regulatory element that is operative in the host cell to allow FL-S to be expressed by the host cell. The regulatory element(s) may allow for constitutive or conditional expression of FL-S. Further, the nucleic acid molecule may be, but need not be, codon-optimized for expression in the host cell. Methods and tools for codon-optimization are well established in the art. In a particular embodiment, the nucleic acid molecule comprises the nucleotide sequence set forth in SEQ ID NO: 3, SEQ ID NO: 6, SEQ ID NO: 9, SEQ ID NO: 12, SEQ ID NO: 15, SEQ ID NO: 17, SEQ ID NO: 19, SEQ ID NO: 21, SEQ ID NO: 23, or SEQ ID NO: 49. In embodiments, the cell may comprise two or more of these sequences, which may be present in a single nucleic acid molecule or in two or more distinct nucleic acid molecules. The nucleic acid molecule(s) may be a plasmid or any other vector suitable to allow expression in an animal cell. Further, the nucleic acid molecule(s) may be extrachromosomal or integrated into the genome of the host cell.

The eVLP, or a plurality of eVLPs, may be present in a composition, such as a pharmaceutical composition, an immunogenic composition, or a vaccine composition, together with a pharmaceutically acceptable carrier or diluent. The composition may further comprise an adjuvant to promote an immune response when the composition is administered to a subject. Suitable adjuvants will be known to one skilled in the art and they include, but are not limited to, aluminum salts, emulsions, and toll-like receptor (TLR) agonists. The composition may be formulated for administration by any suitable route, including parenteral administration and mucosal administration. Vaccine formulations are typically administered orally, intranasally, subcutaneously, or intramuscularly.

The eVLPs and compositions described herein, or eVLPs produced by a method described herein, may be used to induce an immune response to SARS-CoV-2 spike protein, or to prevent COVID-19 or SARS-CoV-2 infection in a subject. Multivariant eVLPs as described herein may be used to induce an immune response to two or more SARS-CoV-2 variants. Multivalent eVLPs as described herein may be used to induce an immune response to SARS-CoV-2 spike protein and/or the at least one additional recombinant protein. Accordingly, the present disclosure includes a method of inducing an immune response or preventing COVID-19 or SARS-CoV-2 infection in a subject by administering to the subject an eVLP or composition as described herein or produced by a method described herein. Further, eVLPs and compositions described herein, or eVLPs produced by a method described herein may be used in the preparation of a medicament for the prevention of COVID-19 or SARS-CoV-2 infection. Prevention may or may not provide complete protection against COVID-19 or SARS-CoV-2 infection. A subject may still become infected and/or exhibit symptoms of the disease, though perhaps with milder (or no) symptoms. Further, the degree of protection may vary across a population, with some members of the population exhibiting a high degree of protection against infection or disease, and other members of the population exhibiting a lesser degree of, or no apparent, protection against infection or disease.

The above disclosure generally describes the present invention. A more complete understanding can be obtained by reference to the following specific examples. These examples are provided solely for the purpose of illustration and they are not intended to limit the scope of the disclosure. Changes in form and substitution of equivalents are contemplated as circumstances might suggest or render expedient. Although specific terms may be employed herein, such terms are intended in a descriptive sense and not for purposes of limitation.

The following non-limiting examples are illustrative of the present disclosure:

### EXAMPLES

### Example 1: Production of Enveloped SARS-CoV-2 Virus-Like Particles

### Materials and Methods

### Cells and Culture Conditions

NRC CHO clones CHO^{55E1} and CHO²³⁵³ have been described previously (Poulain et al, 2017; Isho et al, 2020; Joubert et al, 2023). The endogenous retrovirus-like particle (ERVLP)-deficient CHO-C2 clone was derived from the parental CHO²³⁵³. Cells were maintained in a growth medium in polycarbonate Erlenmeyer flasks with 0.2 μm vent cap (Corning) with constant orbital shaking at 120 rpm in a humidified incubator with 5% CO₂ at 37° C.

### Plasmid Constructs

The coding sequences for SARS-CoV-2 spike(S), matrix (M) and envelope (E) were obtained from the genome reference sequence NC_045512. Unless indicated otherwise, all spike protein sequences contained mutations at the S1/S2 furin cleavage site (R682G, R683G, R685S) and stabilizing proline mutations (K986P, V987P). Expression constructs for variant of concern (VOC) spike proteins were prepared by re-synthesizing and replacing restriction fragments encompassing mutations present in Beta B.1.351 (L18F D80A, D215G, L241del, L242del, A243del, R246I, K417N, E484K, N501Y, D614G, A701V), Delta B.1.617.2 (T19R, G142D, E156del, F157del, R158G, L452R, T478K, D614G, P681R, D950N), and Omicron (A67V, H69del, V70del, T95I, G142D, V143del, Y144del, Y145del, N211del, L212I, E215del, E216del, E217del, F339D, S371L, S373P, S375F, K417N, N440K, G446S, S477N, T478K, E484A, Q493K, G496S, Q498R, N501Y, Y505H, T547K, D614G, H655Y, N679K, P681H, N764K, D796Y, N856K, Q954H, N969K, L981F) variants. Schematics of the general structures of expression constructs described herein are provided in FIG. 6 (not to scale).

Subsequently, coding sequences for reverse mutants of the spike protein were prepared, comprising: (1) mutations at the S1/S2 furin cleavage site (R682G, R683G, R685S) with stabilizing prolines removed (986 KV) (“stabilizing prolines reverse mutant”) or (2) a wild-type S1/S2 furin cleavage site (682RRAR) and stabilizing prolines (K986P, V987P) (“furin cleavage site reverse mutant”).

Sequences for specific SARS-CoV-2 spike proteins described herein are provided in the accompanying electronic sequence listing and summarized in Table 2.

TABLE 2
Summary of sequences for SARS-CoV-2 spike protein constructs
and other sequences referenced in the present disclosure

| Name | SEQ ID NO: | Description/Sequence |
|---|---|---|
| S reference coding sequence | 1 | GenBank Accession NC_045512, nucleotides |
| | | 21563 to 25384 |
| S reference sequence | 2 | GenBank Accession No. YP_009724390.1 |
| | | (excluding the signal peptide (aa 1-13)) |
| FL-S (Wuhan) coding | 3 | Coding sequence for FL-S, codon-optimized |
| sequence | | for expression in mammalian cells |
| FL-S (Wuhan) | 4 | Amino acid sequence for FL-S |
| FL-S (Wuhan), without | 5 | Amino acid sequence for FL-S (Wuhan), |
| stabilizing prolines | | without stabilizing prolines |
| FL-S (Beta) coding sequence | 6 | Coding sequence for FL-S (Beta variant), |
| | | codon-optimized for expression in mammalian |
| | | cells |
| FL-S (Beta) | 7 | Amino acid sequence for FL-S (Beta variant) |
| FL-S (Beta), without | 8 | Amino acid sequence for FL-S (Beta variant), |
| stabilizing prolines | | without stabilizing prolines |
| FL-S (Delta) coding sequence | 9 | Coding sequence for FL-S (Delta variant |
| | | sequence), codon-optimized for expression in |
| | | mammalian cells |
| FL-S (Delta) | 10 | Amino acid sequence for FL-S (Delta variant) |
| FL-S (Delta), without | 11 | Amino acid sequence for FL-S (Delta variant), |
| stabilizing prolines | | without stabilizing prolines |
| FL-S (Omicron) coding | 12 | Coding sequence for FL-S (Omicron variant |
| sequence | | sequence), codon-optimized for expression in |
| | | mammalian cells |
| FL-S (Omicron) | 13 | Amino acid sequence for FL-S (Omicron |
| | | variant) |
| FL-S (Omicron), without | 14 | Amino acid sequence for FL-S (Omicron |
| stabilizing prolines | | variant), without stabilizing prolines |
| FL-S-HA (Wuhan) coding | 15 | HA tagged FL-S (Wuhan), codon-optimized |
| sequence | | for expression in mammalian cells and |
| | | including the added KpnI site |
| FL-S-HA (Wuhan) | 16 | HA tagged FL-S (Wuhan), including the CT |
| | | insertion resulting from the added KpnI site |
| FL-S-HA (Beta) coding | 17 | HA tagged FL-S (Beta variant), codon- |
| sequence | | optimized for expression in mammalian cells |
| | | and including the added KpnI site |
| FL-S-HA (Beta) | 18 | HA tagged FL-S (Beta variant), including the |
| | | CT insertion resulting from the added KpnI |
| | | site |
| FL-S-HA (Delta) coding | 19 | HA tagged FL-S (Delta variant), codon- |
| sequence | | optimized for expression in mammalian cells |
| | | and including an added KpnI site |
| FL-S-HA (Delta) | 20 | HA tagged FL-S (Delta variant), including the |
| | | CT insertion resulting from the added KpnI |
| | | site |
| FL-S-HA (Omicron) coding | 21 | HA tagged FL-S (Omicron variant), codon- |
| sequence | | optimized for expression in mammalian cells |
| | | and including an added KpnI site |
| FL-S-HA (Omicron) | 22 | HA tagged FL-S (Omicron variant), including |
| | | the CT insertion resulting from the added KpnI |
| | | site |
| FL-S-HA (stabilizing prolines | 23 | Coding sequence for HA tagged FL-S |
| reverse mutant) coding | | (stabilizing prolines reverse mutant), codon- |
| sequence | | optimized for expression in mammalian cells |
| | | and including an added KpnI site |
| FL-S-HA (stabilizing prolines | 24 | Amino acid sequence for HA tagged FL-S |
| reverse mutant) | | (stabilizing prolines reverse mutant) |
| FL-S-HA (furin cleavage site | 25 | Coding sequence for HA tagged FL-S (furin |
| reverse mutant) coding | | cleavage site reverse mutant), codon- |
| sequence | | optimized for expression in mammalian cells |
| | | and including an added KpnI site |
| FL-S-HA (furin cleavage site | 26 | Amino acid sequence for HA tagged FL-S |
| reverse mutant) | | (furin cleavage site reverse mutant) |
| Reference coding sequence | 27 | Included in reference sequence, GenBank |
| for S signal peptide | | Accession NC_045512 |
| Codon-optimized coding | 28 | Included in the coding sequences for: FL-S- |
| sequence for S signal peptide | | HA (Wuhan), FL-S-HA (Beta), FL-S-HA |
| #1 | | (Delta), FL-S-HA (stabilizing prolines reverse |
| | | mutant), and FL-S-HA (furin cleavage site |
| | | reverse mutant) |
| Codon-optimized coding | 29 | Included in coding sequence for FL-S-HA |
| sequence for S signal peptide | | (Omicron) |
| #2 | | |
| S signal peptide | 30 | Present in S preprotein prior to signal peptide |
| | | cleavage. May be included in FL-S or FL-S- |
| | | HA preprotein. |
| HA tag coding sequence | 31 | Hemagglutinin (HA) tag coding-sequence |
| | | included in the coding sequences for: FL-S- |
| | | HA (Wuhan), FL-S-HA (Beta), FL-S-HA |
| | | (Delta), FL-S-HA (stabilizing prolines reverse |
| | | mutant), and FL-S-HA (furin cleavage site |
| | | reverse mutant) |
| HA tag | 32 | HA tag amino acid sequence |
| E reference sequence | 33 | GenBank accession no. YP_009724392.1 |
| N reference sequence | 34 | GenBank accession no. YP_009724397.2 |
| M reference sequence | 35 | GenBank accession no. YP_009724393.1 |
| C-terminal 18 amino acids of | 36 | FDEDDSEPVLKGVKLHYT |
| SARS-CoV-2 S | | |
| Influenza A hemagglutinin | 37 | GenBank accession no. EF467821.1 |
| Influenza A neuraminidase | 38 | GenBank accession no. NC_002018.1 |
| Respiratory syncytial virus F | 39 | GenBank accession no. EF566942.1 |
| protein | | |
| Muscarinic acetylcholine | 40 | GenBank accession no. NP_000732.2 |
| receptor M4 | | |
| Galanin receptor type 2 | 41 | GenBank accession no. NP_003848.1 |
| (GALR2) | | |
| FLAG tag | 42 | DYKDDDDK |
| HA tag | 43 | YPYDVPDYA |
| SΔ18 | 44 | 18 aa C-terminal deletion of FL-S (Wuhan) |
| SΔ18 (Beta) | 45 | 18 aa C-terminal deletion of FL-S (Beta) |
| SΔ18 (Delta) | 46 | 18 aa C-terminal deletion of FL-S (Delta) |
| SΔ18 (Omicron) | 47 | 18 aa C-terminal deletion of FL-S (Omicron) |
| S ER retention signal | 48 | KLHYT |
| SΔ18 coding sequence | 49 | Coding sequence for 18 aa C-terminal deletion |
| | | of FL-S (Wuhan), codon-optimized for |
| | | expression in mammalian cells |

All spike gene sequences, codon-optimized for expression in mammalian cells, were synthesized by GenScript, Piscataway, NJ, USA and sub-cloned in digested pTT5® plasmid using EcoRI and BamHI (New England Biolabs). Plasmids were transformed in E. coli DH5a bacteria (Invitrogen) and amplified in CircleGrow® medium (MP Biomedicals) supplemented with 100 μg/mL ampicillin (Gibco). Plasmids were purified using an anion exchanger before ethanol precipitation and sterilization. DNA quantification was carried out using a DeNovix® DS-11+ spectrophotometer. Sequences were verified by Sanger sequencing using an ABI 3500xl genetic analyzer.

### Cell Transfections

CHO cell transfection methods were as described in Stuible et al, 2021. Briefly, CHO^{55E1}, CHO²³⁵³ and CHO-C2 were seeded at 1.8×10⁶ cells/mL 2 days before transfection in the same medium used for cell maintenance. At the time of transfection, cells at ˜6.5×10⁶/mL were diluted with 25% fresh media and dimethylacetamide at 0.083% (v/v). Transfections were carried out with polyethylenimine (PEI)-DNA equivalent to 10% of final volume at 1.4 μg plasmid/mL. PEI-Max (Polyscience) and plasmids are diluted independently in 5% of CHO complete media. Individual structural SARS-CoV-2 gene transfections were performed in CHO complete media using a mixture of 85% pTT5-HA-Spike plasmid, 10% Bcl-XL plasmid (anti-apoptotic effector) and 5% GFP plasmid (by weight). Co-transfection of structural SARS-CoV-2 genes was performed using a mixture of plasmids comprising 42.5% pTT5 Membrane-Envelope (M-E), 42.5% pTT5-HA-Spike, 10% Bcl-XL and 5% GFP diluted in CHO complete media. The diluted PEI-Max was added to the plasmid mixtures and incubated for 7 min at room temperature before adding to cells. Twenty-four hours post-transfection, cells were supplemented with anti-Clumping Supplement (Irvine Scientific) (1:500 dilution), Feed 4 (2.5% v/v), (Irvine Scientific) and shifted to a humidified incubator at 32° C. with 5% CO₂ in constant orbital shaking at 120 rpm. 48 h post transfection, the glucose concentration was measured and adjusted to 40 mM. Supernatants were harvested 5 days post-transfection.

SARS-CoV-2 Virus-Like Particle (eVLP) Harvesting and Sedimentation

Five days post-transfection, cell suspensions (viability ≥95%) were centrifuged for 15 min at 3200 rpm on a Sorvall™ Legend™ RT centrifuge (Thermo). Cell supernatants were kept and pellets discarded. Supernatants were then treated with 10 U/mL of Denarase® (C-LEcta), 5 mM of MgCl₂ and incubated for 1 h at 37° C. For eVLP purification (sedimentation) using iodixanol, treated supernatants were centrifuged over a 15% Optiprep™ (w/v) (Sigma-Aldrich) (de Wit et al, 2013) cushion (10% of total volume) at 5,300 g for 16 h at 4° C. After centrifugation, supernatants were discarded and pellets were resuspended in dPBS (Cytiva).

SARS-CoV-2 eVLP Purification by Affinity Chromatography

Denarase-treated CHO-C2 supernatants were directly purified on AVIPure®-COV2S VLP (AVIpure) resin (Avitide) packed in a Bio-Rad® Poly-Prep® chromatography column. AVIpure column was first equilibrated with 5 column volumes (CV) of dPBS. Next, treated supernatant was loaded at a constant flow rate of 1 mL/min. The column was then washed with 5 CV of dPBS. eVLPs were eluted with 50 mM Bis-Tris, 1 M MgCl₂. pH 6.0 followed by desalting into dPBS using NAP25 columns. Desalted eVLPs were filter-sterilized and tested for endotoxins (Endosafe®-PTS cartridges, Charles River) prior to in vivo experiments.

### Protein Quantification

Precipitated or purified eVLPs were subjected to protein quantification using the Pierce bicinchoninic acid (BCA) protein assay (Thermo Fisher Scientific). In brief, 200 μL of BCA working reagent prepared according to the manufacturer's protocol was added to 96-well plate and mixed with 25 μL of bovine serum albumin (BSA) standards (Thermoscientific), sedimented eVLPs, or purified eVLPs and incubated for 30 min at 37° C. Absorbance at 540-590 nm was measured using a SpectraMax® 340PC spectrophotometer.

### SDS-PAGE and Immunoblotting

Following protein quantification, 0.7 μg of total protein from eVLPs or 7.5 μl of denarase-treated supernatants were heat-denatured at 70° C. for 10 min under reducing conditions followed by separation of proteins by SDS-PAGE using NuPAGE™ 4-12% Bis-Tris gels (Invitrogen). Total protein staining (Coomassie Blue) was performed using standard methods. Spike concentration was determined by densitometry, in comparison to a reference recombinant soluble spike trimer (NRC reference material SmT1 (Stocks et al, 2022)) standard curve using a ChemiDoc™ MP imaging system (Bio-Rad) and Image Lab 6.1.

For immunoblotting, proteins separated by SDS-PAGE 4-12% were transferred to nitrocellulose membranes using a Trans-Blot Turbo (Bio-Rad) apparatus with default settings. Membranes were blocked for 30 min with 5% milk in dPBS with 0.1% Tween® 20 (dPBS-T) followed by three 5-minute washes with dPBS-T. Membranes were then incubated for 1 h at room temperature with anti-HA (Sigma H6908), anti-Matrix (ProSci 3529) or a mouse anti-RVLP-Gag p30 (produced in-house) at a 1:1000 dilution. Primary antibody incubation was followed by three 5-minute washes with dPBS-T and incubation for 1 h with horseradish peroxidase-conjugated secondary antibodies at a 1/10,000 dilution for goat anti-rabbit (Jackson 111-035-003) and 1/5000 dilution for goat anti-mouse (Sigma A2304). After three additional 5-minute washes with dPBS-T, the bands were visualized with Clarity™ Western ECL Substrate (Bio-Rad) and ChemiDoc MP imaging system.

Negative-Stain Transmission Electron Microscopy of eVLPs

Observation of eVLPs by a transmission electron microscope (TEM, HITACHI H-7500) equipped with bottom-mounted AMT NanoSprint 12MP camera and operating at 80 k V in high-contrast mode, was performed using a negative-staining. TEM grids (Cu 200 mesh, 15-25 nm carbon supported) were freshly glow-discharged using EMS GloQube®-D, Dual chamber glow discharge system (Electron Microscopy Sciences) in negative mode with plasma current of 25 mA during 45 s. Such grids were floated on 10 μl sample aliquots on Parafilm™ for 5 min. The excess droplets were subsequently wicked away from the edge of the grid with the filter paper strips (Whatman™ 541). The grid was then rinsed three times with three droplets of double distilled water each time removing the excess. Immediately after last rinse, the grid was exposed to the staining solution (optimized to be 2% formic acid) for 60 s and the stain was carefully removed using a fresh piece of filter paper. Finally, the grid was dried at the ambient conditions for 2 h and used for TEM analysis.

Cryo-Electron Microscopy of eVLPs

FL-S eVLP samples were prepared by cryo-plunge (Dobro et al, 2010) with a Leica-EM GP2 plunger. TEM grids (Cu 400 mesh, with holey carbon film) were treated with a Harrick PDC-32G plasma cleaner with a plasma power of 18 W for 60 s. Such grids were picked up with adapter-attached forceps and loaded in an environmental chamber (humidity ˜80%) of the Leica-EM GP2 unit. 5 μL of aqueous sample was applied on the carbon-film side of TEM gird and blotted with a filter paper (Whatman™ #1) for 1.5 s. The grid was then plunged into liquid ethane with a temperature of −180° C., and transferred to a cryo-sample box in liquid nitrogen (LN2). The sample box was further transferred in LN2 and loaded to a pre-cooled (−180° C.) Gatan 914 cryo-specimen holder in a workstation filled with LN2. The holder with the cryo-grid protected under an anti-frost cover was inserted in the TEM column. Observation of the cryo-plunged eVLPs sample was carried out on a JEOL 2200 FS TEM, operating at 200 kV, equipped with a field emission filament. Image collections were under low dose mode (Sun & Li, 2010) to migrate beam damage. A 10 eV wide energy filter (with only zero energy loss electrons contributing for image formation) was used to enhance image contrast. Each image was collected with exposure time between 0.5˜1 s based on the local vitreous ice thickness and the microscope settings. Images were processed with the build-in functions (smooth, contrast reversal) of Gatan Digitalmicrograph™ software.

### Nanoparticle Tracking Analysis (NTA)

FL-S eVLP purified material and mock samples contained in dPBS were analyzed by NTA with a NanoSight® NS 5000 (Malvern Panalytical) equipped with a laser wavelength of 532 nm and a long-pass filter long 565 nm. Two sets of data were acquired. Parameters for NTA measurements of samples were performed as follows: eVLPs and polystyrene beads were captured 3 times (1 independent preparation) at a temperature of 20° C. with the camera Level at 16 (Slider Shutter 1300; Slider Gain 512). The following analysis settings were used to process the acquired data; Detection Threshold 3; Blur Size and Max Jump Distance were Auto. eVLP samples were diluted in dPBS at a minimal final volume of 1 mL to allow replicate injection. Aspiration of the sample into the system was done using the integrated peristaltic-pump connected to a tubing. A minimal volume of 1.8 mL was prepared for each sample to allow technical replicate analysis. Nanosight NS 5000 calibration was carried out by loading 100 nm polystyrene beads, 1/100,000 (ThermoFisher) with a predetermined acceptance criteria of 15 to 90 particles per frame and a ≤15% of CV between injections from same preparation.

Mock samples were diluted in dPBS at 1:475. FL-S eVLPs were diluted at 1:500, 1:1000, 1:1250, 1:1500 and 1:2000. Image processing, particle size distribution and concentration calculations were performed with Nanosight NTA 3.2 Analytical Software (Malvern Panalytical) with a FTLA fitting model.

eVLP Formation and Release

Five days post-transfection using a FL-S-HA construct (R682G, R683G, R685S, K986P, V987P) or reversed mutants (682 RRAR>GGAS) or (986 KV>PP) in CHO-C2, cell supernatants were harvested, treated and sedimented as described above. The pellet was resuspended in 1.5 mL of dPBS prior to further analysis. 12 μl of the resuspended sedimented supernatants were heat-denatured at 70° C. for 10 min under reducing conditions followed by separation of proteins by SDS-PAGE using NuPAGE 4-12% Bis-Tris gels (Invitrogen). Total protein staining (Coomassie Blue) was carried out using standard methods. Protein staining was visualized using a ChemiDoc MP imaging system (Bio-Rad) and Image Lab 6.1.

For immunoblotting, proteins contained in 12 μl of the resuspended sedimented supernatants were equally separated by SDS-PAGE 4-12% and transferred to nitrocellulose membranes using a Trans-Blot Turbo (Bio-Rad) apparatus with default settings. Membranes were blocked for 30 min with 5% milk in dPBS with 0.1% Tween 20 (dPBS-T) followed by three 5-minute washes with dPBS-T. Membranes were then incubated for 1 h at room temperature with anti-S1 (ProSci #9083) at a 1:1000 dilution. Primary antibody incubation was followed by three 5-minute washes with dPBS-T and incubation for 1 h with a secondary anti-rabbit (Jackson 111-035-003) at 1/10,000 dilution. After three additional 5-minute washes with dPBS-T, the bands were visualized with Clarity Western ECL Substrate (Bio-Rad) and a ChemiDoc MP imaging system.

In addition, the eVLPs produced by expressing the FL-S-HA construct (R682G, R683G, R685S, K986P, V987P) or the reversed mutant constructs (682 RRAR>GGAS) or (986 KV>PP) in CHO-C2 were imaged by negative-stain transmission electron microscopy, as described above.

### Results

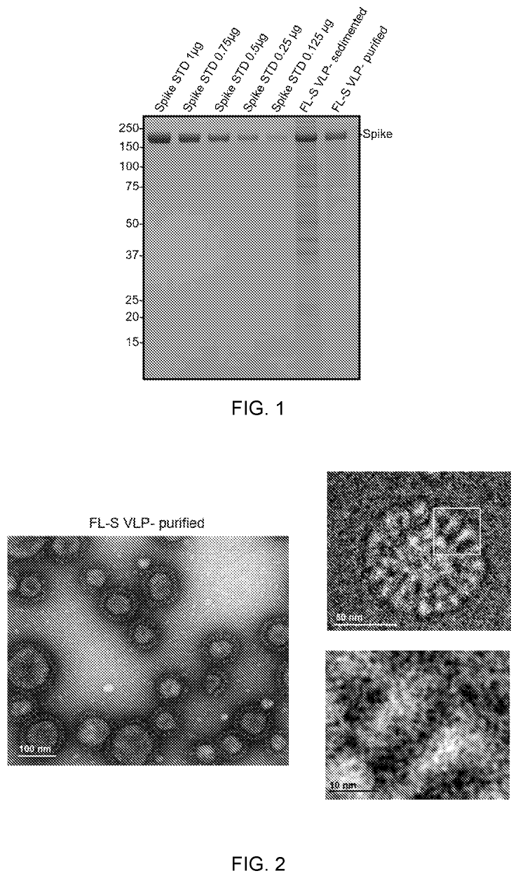

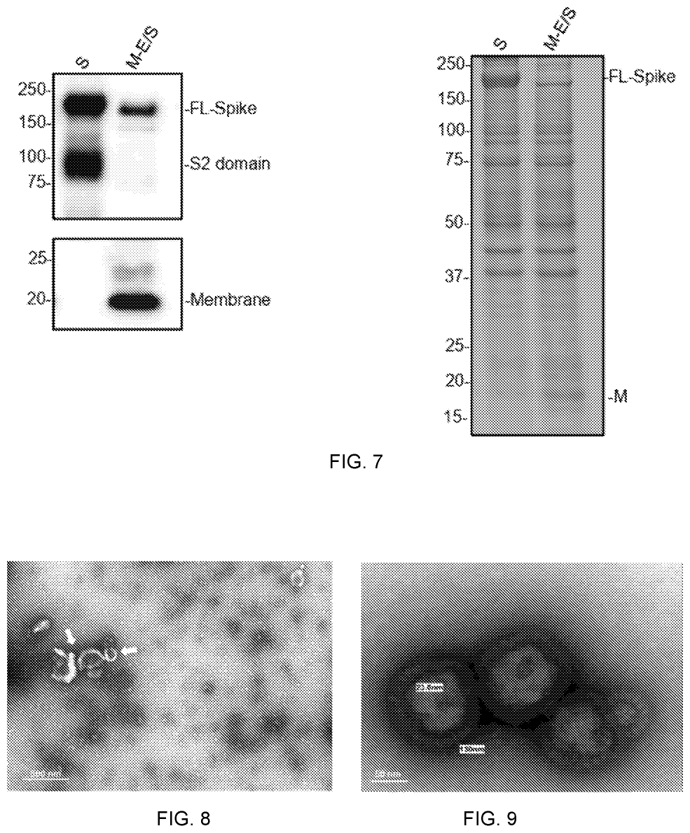

Chinese Hamster Ovary (CHO) cells are the predominant mammalian host cell for industrial therapeutic recombinant protein manufacturing and CHO cells are capable of producing eVLPs, but they have not been used for this purpose to any significant extent. Given their potential advantages for eVLP manufacturing, we first explored the structural protein co-expression approach (Xu et al, 2020; Swann et al, 2020) in CHO cells (FIGS. 1-5). As shown in FIG. 7 (left), when M, E and S are co-expressed, we observe the presence of M and S in cell culture supernatants by Western blotting, consistent with release of these proteins from cells in eVLPs. However, when supernatants were subjected to ultracentrifugation on an iodixanol cushion to enrich for sedimentable particles (FIG. 7, right), negative-stain transmission electron microscopy (TEM) imaging failed to detect significant numbers of enveloped particles and very few exhibited the presence of S protein characteristic of SARS-CoV-2 (FIG. 8).

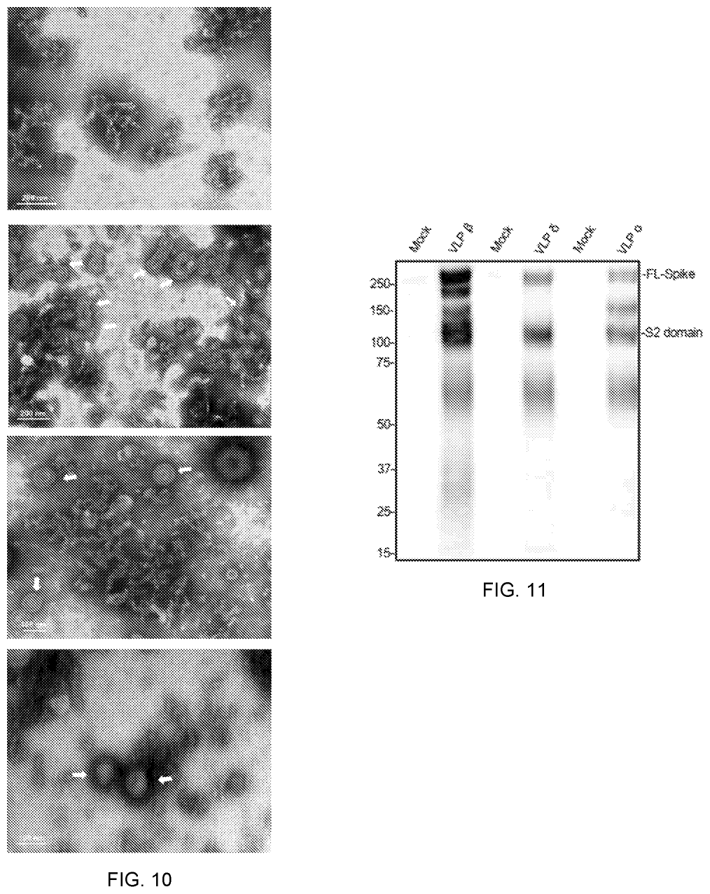

Remarkably, when an FL-S-HA construct was expressed alone, substantially higher levels of spike were released from CHO cells, and this protein could be sedimented by ultracentrifugation on an iodixanol cushion (FIG. 7), suggesting formation of eVLPs. Strikingly, in the iodixanol-pelleted material, we observed by TEM the presence of a very high density of well-ordered, generally spherical structures, averaging 130 nm in diameter, surrounded by a dense coat of spike protein reminiscent of SARS-CoV-2 virions (FIG. 9). Particles with similar characteristics were generated by expression of spike constructs corresponding to the reference (Wuhan) strain or different variants of concern (VOCs) (FIGS. 10 and 11).

Formation of such particles, which we refer to as “FL-S eVLPs”, has not been described previously with expression of full-length spike protein alone in any cell type, and their apparent ease of production in CHO cells led us to proceed with additional characterization, purification method development, and assessment as a SARS-CoV-2 vaccine antigen.

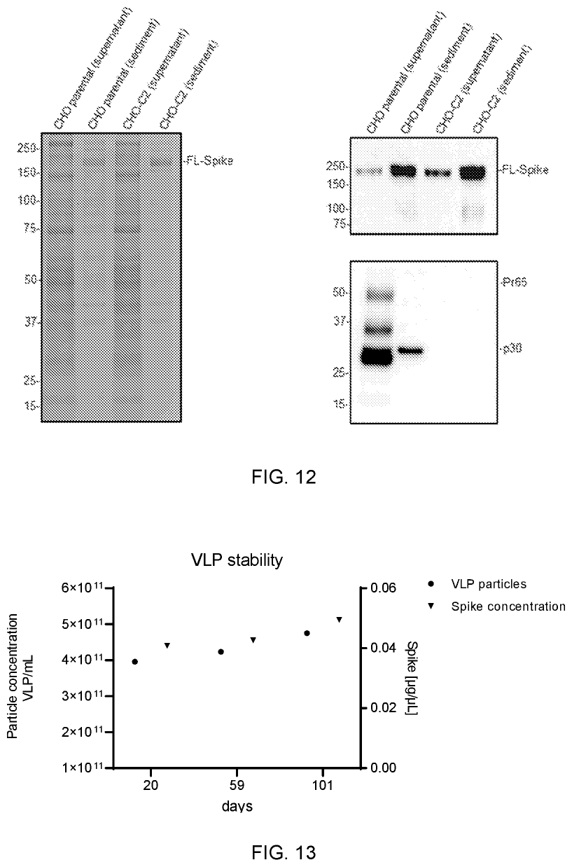

The production of endogenous retrovirus-like particles (RVLPs) by CHO cells, an issue known for several decades (Lieber et al, 1973), would complicate downstream processing of recombinant VLPs. Viral clearance and inactivation steps during recombinant protein purification mitigate the safety risk of RVLPs and other viruses, but these processes would likely disrupt eVLPs as well. CRISPR-Cas9 has been used to target RVLP proviral elements in the CHO genome (Duroy et al, 2020), and we used a similar approach to engineer an RVLP-free CHO cell line (CHO-C2). CHO-C2 cells give FL-S eVLP yields similar to the parental cell line, but without RVLP co-purification, as indicated by anti-RVLP Gag western blotting (FIG. 12).

It is generally challenging to develop purification strategies to separate recombinant eVLPs from other enveloped particles (e.g., exosomes and extracellular vesicles) spontaneously released from production host cell lines. For FL-S eVLP purification, we tested conventional column chromatography resins (ion exchange, lectins, heparin, and hydroxyapatite) with unsatisfactory results before proceeding to evaluate affinity chromatography options, including commercial spike-affinity resins (e.g., Repligen or Avitide) and in-house preparations of beads conjugated to spike antibodies or recombinant ACE2. These resins effectively bound eVLPs, but elution under non-denaturing conditions was inefficient. To address this issue, a novel spike affinity resin with lower ligand density was generated by Avitide, AVIPure® COV2S, that is selective for the SARS-CoV-2 spike protein receptor binding domain. This resin allowed effective capture and gentle elution of intact eVLPs, which could be buffer-exchanged and sterilized by filtration for in vivo testing. Compared to iodixanol-sedimented samples, the affinity-purified FL-S eVLPs contain lower levels of host cells proteins (HCP) by SDS-PAGE and visibly less non-eVLP particulate contaminants by TEM (FIG. 1).

Our current transient CHO production method followed by one-step affinity purification yields ˜1.8 mg/L of FL-S eVLPs, based on spike protein content or 1.43×10¹³ eVLPs/L based on NTA particle counts (Table 3). Of note, our purified eVLP material can be stored at 4° C. for 101 days with negligible loss of spike protein or change in eVLP morphology by TEM (FIG. 13, Table 3).

TABLE 3
Nanoparticle tracking analysis (NTA) to assess FL-S eVLP
stability at different time points after batch production

| | | | Average eVLP | Median eVLP |
|---|---|---|---|---|
| Day | Sample | eVLPs/mL | Size (nm) | Size (nm) |
| 20 | FL-S eVLP | 3.96E+11 | 143 | 128 |
| 59 | FL-S eVLP | 4.24E+11 | 128 | 119 |
| 101 | FL-S eVLP | 4.75E+11 | 110 | 125 |

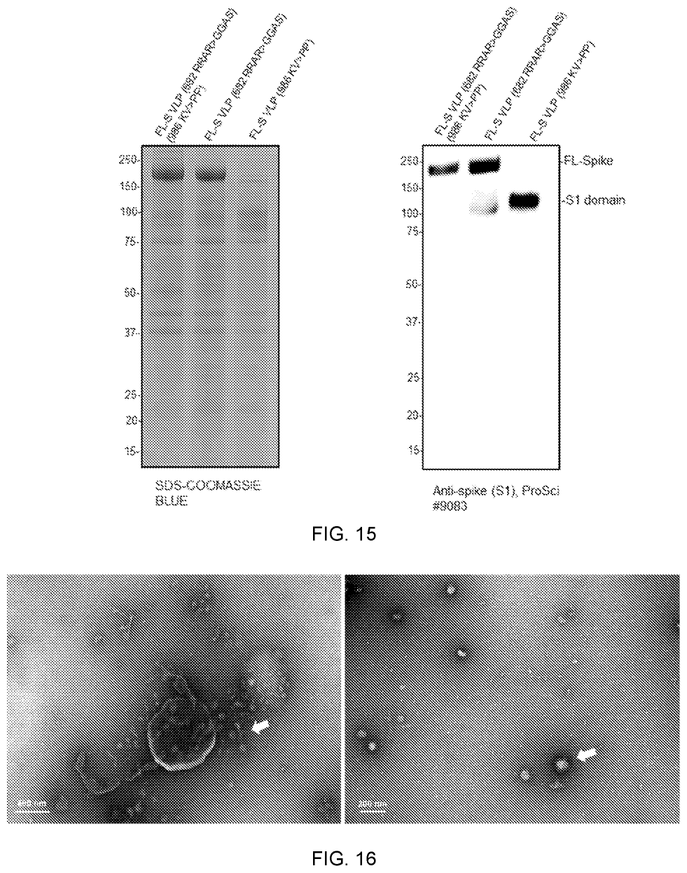

Expressing the FL-S-HA “stabilizing prolines reverse mutant” (SEQ ID NO: 23) and the FL-S-HA “furin cleavage site reverse mutant” (SEQ ID NO: 25) in CHO cells demonstrated that a high concentration of the FL-spike protein can be achieved regardless of the presence or absence of stabilizing prolines (986 KV>PP) (see the left two lanes in the SDS PAGE gel and Western blot shown in FIG. 15), but that high levels of FL-S expression are dependent on inactivation of the S1/S2 furin cleavage site (see the rightmost lane in the SDS PAGE gel and Western blot shown in FIG. 15). TEM images confirmed the formation of eVLPs in high quantities in “FL-S stabilizing prolines reverse mutant” samples (FIG. 16). Conversely, the “FL-S furin cleavage site reverse mutant” samples did not have detectable levels of the full-length spike protein, with concomitant scarce production of FL-S eVLPs (FIGS. 15 and 16). This suggests that an inactivated S1/S2 furin cleavage site is necessary for high levels of FL-S production and, correspondingly, for high levels of FL-S eVLP production.

### Example 2: Immunization of Mice with SARS-CoV-2 eVLPs

### Materials and Methods

### Mice

Female C57BL/6 mice (6-8 weeks old) were purchased from Charles River Laboratories (Saint-Constant, Canada). Animals were maintained at the animal facility of the National Research Council Canada (NRC) in accordance with the guidelines of the Canadian Council on Animal Care. All procedures performed on animals in this study were approved by the NRC's Institutional Review Board (NRC Human Health Therapeutics Animal Care Committee) and covered under animal use protocol 2020.10. All experiments were carried out in accordance with the ARRIVE guidelines.

### Mice Immunization and Sample Collection

Eight groups (n=10 per group) of mice were immunized with different formulations to evaluate the immunogenicity of FL-S eVLPs. Likewise, seven other groups of mice were immunized with a soluble trimeric spike (ECDm-T4-Fib) previously described by Stuible et al., (2021) formulated in the same way. In brief, affinity purified FL-S eVLPs produced as described in Example 1, or purified standard ECDm-T4-Fib, along with adjuvant vaccine components were admixed and diluted in PBS (Thermo Fisher Scientific) prior to administration in a final volume of 50 μL per dose. Adju-Phos® (Invivogen) dose levels were based on data from previous studies with 50 μg A13+ included per dose (Akache et al, 2021). Adju-Phos® is an aluminum phosphate wet gel suspension. AS01b (GlaxoSmithKline, Brentford, UK) based on the saponin QS-21 and TLR4 agonist monophosphoryl lipid A (MPL) was used at 1/20 of human dose.

Animals were immunized by intramuscular (i.m.) injection (50 μL) into the left tibialis anterior (T.A.) muscle on days 0 and 21 with vaccine formulations of 3 μg of FL-S eVLPs or ECDm-T4-Fib alone, and 3 μg, 0.3 μg and 0.06 μg of FL-S eVLPs or ECDm-T4-Fib with adjuvant (eVLP quantities are based on the amount of spike protein present in the purified particles). Adjuvant amounts were kept constant regardless of antigen dose. Mice were bled via the submandibular vein on days 20 and 28 and recovered serum was used for quantification of antigen specific IgG antibody levels and neutralization assays. On day 28, mice were anesthetized with isoflurane and then euthanized by cervical dislocation prior to collection of spleens for measurement of cellular immune responses by IFN-γ ELISpot. For comparative purposes with a single dose, animals injected with ECDm-T4-Fib were serum analyzed (IgG antibody levels and surrogate neutralization assays) at day 20.

### Mouse Serum Anti-Spike ELISA

The method to determine total anti-spike IgGs has been described previously (Akache et al, 2021). Briefly, 96-well high-binding ELISA plates (Thermo Fisher Scientific) were incubated overnight at room temperature (RT) with 100 μL of 0.3 μg/mL SmT1 (Stocks et al, 2022) spike protein diluted in PBS. Plates were washed five times with PBS-T 0.05% and blocked for 1 hour at 37° C. with 200 μL of PBS-fetal bovine serum (FBS) 10% (Thermo Fisher Scientific). The plates were washed five times with PBS-T 0.05%. Next, 3.162-fold serially diluted samples in PBS-T 0.05% with 10% FBS were added in 100 μL volumes and incubated for 1 hour at 37° C. After five washes with PBS-T 0.05%, 100 μL of goat anti-mouse IgG-HRP (1:4,000, Southern Biotech) or goat anti-hamster IgG-HRP (1:32,000, Southern Biotech) was added for 1 hour at 37° C. After five further washes with PBS-T 0.05%, 100 μL/well of the substrate o-phenylenediamine dihydrochloride (OPD, Sigma-Aldrich) diluted in 0.05 M citrate buffer (pH 5.0) was added. Plates were developed for 30 minutes at RT in the dark. The reaction was stopped with 50 μL/well of 4N H₂SO₄. Bound IgG Abs were detected spectrophotometrically at 450 nm. Titers for IgG in serum were defined as the dilution that resulted in an absorbance value (OD 450) of 0.2 and were calculated using XLfit software (ID Business Solutions). Samples that did not reach the target OD were assigned the value of the lowest tested dilution (i.e., 10) for analysis purposes.

### Cell-Based Surrogate SARS-CoV-2 Neutralization Assay

The surrogate neutralization assay has been described elsewhere (Wrapp et al, 2020a). In brief, the degree of binding of soluble spike protein to the surface of HEK-293T or Vero E6 cells overexpressing human ACE2 is measured following co-incubation of the protein with sera/plasma. HEK-293T cells were maintained in D-MEM high glucose (Gibco) with 10% FBS and 1% penicillin/streptomycin (from Thermo Fisher Scientific). Vero E6 cells were maintained in RPMI 1640 supplemented with 10% FBS, 1% penicillin/streptomycin, 20 mM HEPES, 1× non-essential amino acids, 1× Glutamax, 50 μM 2-mercaptoethanol (all from Thermo Fisher Scientific) at 37° C. with 5% CO₂. Soluble spike protein (SmT1) was biotinylated using EZ-Link™ NHS-LC-LC-Biotin (Thermo Fisher Scientific) according to manufacturer's instructions. Indicated dilutions of mouse sera were mixed with 250 ng of biotinylated spike and 1×10⁵ Vero E6 cells in the presence of 0.05% azide in a 96-well V-bottom plate (Nunc, Thermo Fisher Scientific) and incubated for 1 hour at 4° C., in darkness. Regardless of serum concentration, the final volume of all samples was normalized to 150 μL. Cells were washed with PBS-1% BSA+0.05% azide and incubated with Streptavidin-phycoerythrin conjugate for 1 hour at 4° C. (Thermo Fisher Scientific). After an extra wash, cells were fixed using CytoFix™ (Becton Dickinson) and resuspended in wash buffer +5 mM EDTA for acquisition on an LSR Fortessa™ (Becton Dickinson). Spike bound to cells was determined by calculating the geometric Mean Fluorescence Intensity (gMFI) of PE (on singlet cell population) and subtracting the same parameter measured from control cells (incubated in absence of plasma/serum), both above background noise as determined by the negative control, using FlowJo™ analysis software. Percent neutralization was calculated with the next equation according to (Akache et al, 2021):

$$\% neutralization = \frac{100 - [ 100 \times ( gMFI of PEsample - gMFI of PEnegative control ) ]}{gMFI of PEpositive control - gMFI of PEnegative control}$$

Samples with values≤0 were considered 0.

### ELISpot

The levels of spike glycoprotein-specific T cells were quantified similarly as in (Akache et al, 2021) by ELISpot in a mouse IFN-γ kit (Mabtech Inc). Spleens were mechanically minced and splenocytes were isolated in RPMI containing 10% FBS, 1% penicillin/streptomycin (Thermo Fisher Scientific), 1% glutamine (Thermo Fisher Scientific) and 55 μM 2-Mercaptoethanol (Thermo Fisher Scientific). Cells were strained using 70-μm filters and cell concentrations were determined on a Cellometer® (Nexcelom). A spike peptide library (JPT Peptide Technologies GmbH) consisting of 315 peptides (15mers overlapping by 11 amino acids with last peptide consisting of a 17mer) was used to stimulate the cells. The library was split into 3 sub-pools, each covering a third of the spike protein, which were used to separately stimulate 4×10⁵ cells in duplicate at a final concentration of 2 μg/mL per peptide. Cells were also incubated without any stimulants to measure background responses. After an incubation of ˜20 hours at 37° C. with 5% CO₂, plates were washed and developed according to the manufacturer's protocol. AEC substrate (Becton Dickenson) was used to visualize the spots. Spots were counted using an automated ELISpot plate reader (Cellular Technology LTD). For each animal, values obtained with media alone were subtracted from those obtained with each of the spike peptide pools, and then combined to yield an overall number of antigen-specific IFN-γ+SFC/10⁶ splenocytes per animal.

### Results

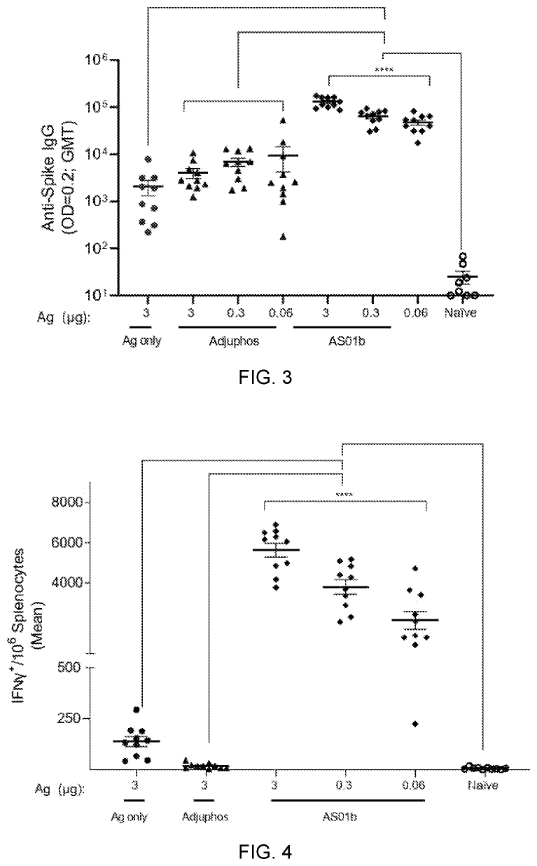

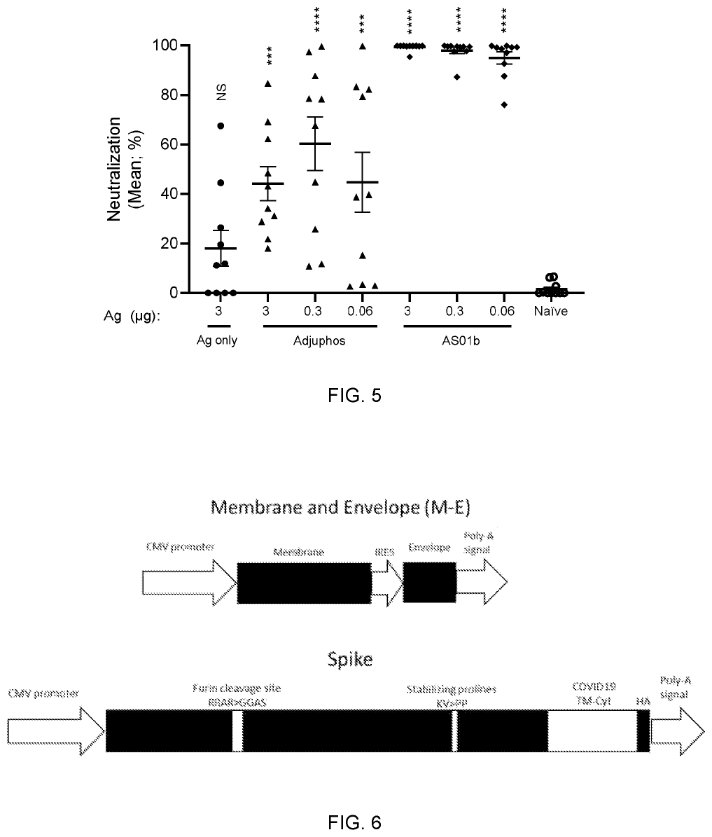

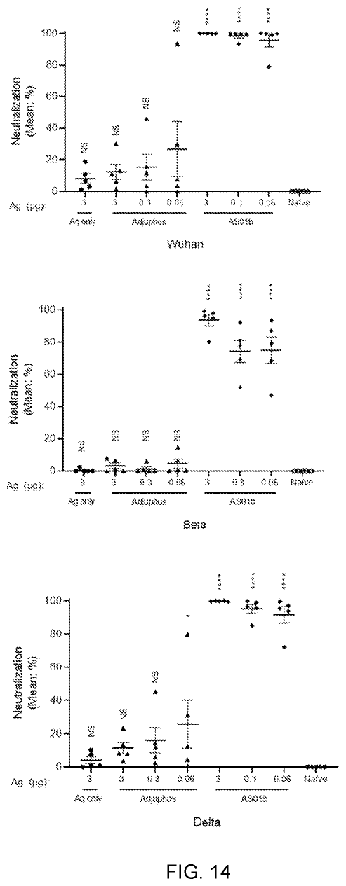

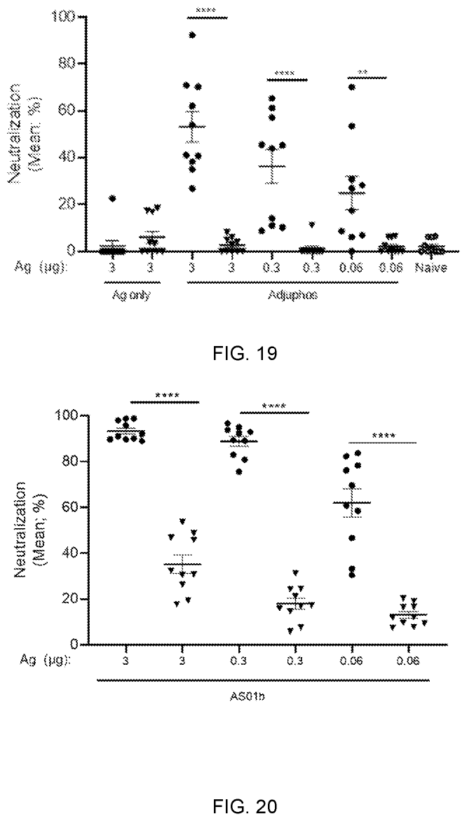

To assess the potential of FL-S eVLPs as a vaccine antigen in vivo, mice (n=10/per group) were immunized at day 0 and boosted on day 20 with eVLPs alone (containing 3 μg spike protein), or with different doses (3 μg, 0.3 μg or 0.06 μg) of spike protein with Adju-Phos® or AS01b adjuvants. Likewise, and for comparative purposes with a single dose, we immunized other groups of mice (n=10/per group) at day 0 with the soluble trimeric spike ECDm-T4-Fib (Stuible et al., 2021) using the same dose levels and adjuvants. Analysis of day 28 serum samples demonstrated that eVLPs induce anti-spike antibodies even without adjuvant, but as low as 60 ng of eVLPs induce titers 11× or 23× higher than the antigen alone when adjuvanted with Adju-Phos® or AS01b (p<0.0001) (FIG. 3). IFN-γ ELISpot showed that specific T cell responses are strongly elicited by eVLPs adjuvanted with AS01b at all doses tested (p<0.0001), moderately by antigen alone, but inhibited when combined with Adju-Phos® (FIG. 4). The surrogate neutralization assay for Wuhan demonstrated overall that serum from eVLPs-alone and eVLPs+Adju-Phos groups had substantial variability in neutralization activity (FIG. 5). In contrast, serum from the eVLPs+AS01b group, as shown also in FIG. 5, topped out neutralizing titers (p<0.0001). Additional studies for VOCs demonstrated a similar neutralization activity in eVLPs+AS01b (p<0.0001) mice even with the lowest (60 ng) dose tested (FIG. 14). We further compared the humoral response between FL-S eVLPs and the ECDm-T4-Fib to elucidate if there were potential advantages for eVLPs over the trimeric spike when used at a single dose. As shown in FIG. 18, FL-S eVLPs induce a higher concentration of anti-spike antibodies in the presence or absence of adjuvants. Additional surrogate neutralization assays for Wuhan demonstrated a higher induction of neutralizing antibodies by eVLPs than the soluble spike with Adju-Phos® (FIG. 19) and maximal neutralizing titers (p<0.0001) when adjuvanted with AS01b (FIG. 20).

In conclusion, we discovered that FL-S expressed in CHO cells is sufficient to induce assembly and release of abundant, high-density spike-coated eVLPs. Compared to other VLP technologies, these eVLPs are easier to produce using a well-established host cell line compatible with large-scale manufacturing, are primarily composed of spike protein (unlike Gag VLPs) and can be efficiently purified using a simple one-step process. We show that nanogram doses, significantly lower than reported for any other purified SARS-CoV-2-antigen vaccines, of FL-S eVLPs, when adjuvanted with AS01b, elicit a potent cellular and humoral response in mice. This new technology could greatly facilitate manufacturing of a highly immunogenic SARS-CoV-2 vaccine candidate to help drive accessibility of booster shots worldwide.

### Example 3: Effect of the Spike Protein C-Terminal Tail on VLP Production

### Materials and Methods

### Cells and Culture Conditions

As described in Example 1.

### Plasmid Constructs

The coding sequence for SARS-CoV-2 S, was obtained from the genome reference sequence NC_045512. All full-length S protein sequences contained mutations at the furin cleavage site (R682G, R683G, R685S), stabilizing prolines (K986P, V987P) and C-terminal HA epitope-tag (YPYDVPDYA) unless otherwise specified. The Sm-delta18 S (SΔ18) protein (SEQ ID NO: 44) contains an 18 amino acid C-terminal deletion (deletion of FDEDDSEPVLKGVKLHYT, SEQ ID NO: 36), starting at 1257 to 1275 aa position related to the full-length S Wuhan strain described above.

Plasmids were transformed in E. coli DH5α bacteria (Invitrogen) and amplified in CircleGrow medium (MP Biomedicals) supplemented with 100 μg/mL ampicillin (Gibco). Plasmids were purified using a proprietary anion exchange chromatography method followed by isopropanol precipitation, ethanol wash and 0.22 μm filtration. DNA quantification was carried out using a DeNovix DS-11+ spectrophotometer. Sequences were verified by Sanger sequencing using an ABI 3500xl genetic analyzer.

### Cell Transfections

CHO cell transfection methods were performed as described in Example 1. Individual SARS-CoV-2 gene transfections were performed in CHO media at 85% of pTT5-Spike-HA or pTT5-Spike (Δ18), 10% Bcl-XL plasmid (anti-apoptotic effector) and 5% GFP plasmid. Co-transfection of structural SARS-CoV-2 genes for bivariant VLPs was performed using a mixture of plasmids comprising 59.5% pTT5-Spike (Δ18), 25.5% pTT5-Spike (B.1.1.529)-HA or 25.5% pTT5-Spike (BA4/BA5))-HA, 10% Bcl-XL and 5% GFP diluted in CHO complete media.

### SARS-CoV-2 VLP Harvesting and Sedimentation

Five days post-transfection, cell suspensions (viability ≥95%) were centrifuged for 15 min at 3200 rpm on a Sorvall legend RT centrifuge (Thermo). Cell supernatants are kept and pellets discarded. Supernatants were then treated with 10 U/mL of Denarase (C-LEcta), 5 mM of MgCl2 and incubated for 1 h at 37° C. For VLP sedimentation, treated supernatants were centrifuged over a 15% Optiprep (w/v) (Sigma-Aldrich) (de Wit et al., 2013) cushion (10% of total volume) at 5,300 g for 16 h at 4° C. After centrifugation, supernatants were discarded and pellets resuspended in dPBS (Cytiva).

### SARS-CoV-2 VLP and S Purification by Affinity Chromatography

Denarase-treated CHO-C2 supernatants were directly purified on AVIPure-COV2S VLP (AVIpure) resin (Avitide) packed in a BioRad PolyPrep chromatography column. AVIpure column was first equilibrated with 5 column volumes (CV) of dPBS. Next, the treated supernatant was loaded at a constant flow rate of 1 mL/min. The column was then washed with 5 CV of dPBS. VLP were eluted with 50 mM Bis-Tris, 1M MgCl2. pH 6.0 followed by desalting into dPBS using NAP25 columns. Desalted VLPs were filter-sterilized and tested for endotoxins (Endosafe-PTS cartridges, Charles River) prior to in vivo experiments. The SmT2v3 soluble S ectodomain was produced from stable CHO cells as described previously (Colwill et al., 2022) and purified using the NGL COVID-19 S Affinity resin (Repligen) following the manufacturer's instructions.

### Total Protein Quantification

Sedimented or purified VLPs were lysed with RIPA buffer (150 mM NaCl, 1% NP-40, 0.5% sodium deoxycholate, 0.1% SDS, 50 mM Tris-HCL pH 7.4) supplemented with protease inhibitor cocktail (complete, EDTA-free, Roche). VLP samples in RIPA buffer were incubated at 4° C. for 20 min followed by 15 min centrifugation at top speed in a benchtop centrifuge (Eppendorf 5427R). Pellets were discarded and supernatants recovered. Protein quantification was performed with Pierce bicinchoninic acid (BCA) protein assay (Thermo Fisher Scientific). In brief, 200 μL of BCA working reagent prepared according to the manufacturer's protocol was added to 96-well plate and mixed with 25 μL of bovine serum albumin (BSA) standards (Thermo Fischer Scientific), sedimented or purified VLP lysates and incubated for 30 min at 37° C. Absorbance at 562 nm was measured using a Spectra max 340PC spectrophotometer.

### SDS-PAGE and Immunoblotting

Following protein quantification, 0.7-1 μg of total protein from VLPs or 7.5 μL of Denarase-treated supernatants (unless otherwise indicated) were mixed (3:1 [v:v]) with XT sample buffer 4× (BioRad) supplemented with 200 mM dithiothreitol (DTT), heat-denatured at 70° C. for 10 min under reducing conditions followed by separation of proteins by SDS-PAGE using NuPAGE 4-12% Bis-Tris gels. Total protein staining (Coomassie Blue) was performed using standard methods. VLP S protein concentration was determined by the comparison to an NRC Metrology soluble S trimer standard (STD) reference material SMT1-1 (Stocks et al., 2022) using a ChemiDoc MP imaging system (Bio-Rad) and Image Lab Ink 6.1. H1 protein concentration was determined by the comparison to an in-house H1 tagged purified standard.

For immunoblotting, proteins separated by SDS-PAGE were transferred to nitrocellulose membranes using a Trans-Blot Turbo (Bio-Rad) apparatus with default settings. Membranes were blocked for 30 min with 5% milk in dPBS with 0.1% Tween 20 (dPBS-T) followed by three 5-minute washes with dPBS-T. Membranes were then incubated for 1 h at room temperature with anti-S(S1) (ProSci #9083), Anti-S—NRC-S2A4, anti-HA (#H6908 sigma), anti-H1 (Santa Cruz IV.C102, #sc-80550), in-house anti-RSV—F at a 1:1000 dilution and anti-N1 (R&D AF4858) at 1:2000 dilution. Primary antibody incubation was followed by three 5-minute washes with dPBS-T and incubation for 1 h with horseradish peroxidase-conjugated secondary antibodies at a 1/10000 dilution for goat anti-rabbit (Jackson 111-035-003) and 1/5000 dilution for goat anti-mouse (Sigma A2304), donkey anti-sheep (Sigma A3415) and goat anti-human FC (Sigma A0170). After three additional 5-minute washes with dPBS-T, the bands were visualized with Clarity Western ECL Substrate (Bio-Rad) and ChemiDoc MP imaging system.

### Negative-Stain Transmission Electron Microscopy (TEM)

Observation of VLPs by transmission electron microscope (TEM, HITACHI H-7500) equipped with bottom-mounted AMT NanoSprint 12MP camera and operating at 80 kV in high-contrast mode, was performed using negative staining. TEM grids (Cu 200 mesh, 15-25 nm carbon supported) were freshly glow-discharged using EMS GloQube-D, Dual chamber glow discharge system (Electron Microscopy Sciences) in negative mode with plasma current of 25 mA during 45 s. Such grids were floated on 10 μL sample aliquots on Parafilm for 5 min. The excess droplets were subsequently wicked away from the edge of the grid with filter paper strips (Whatman 541). The grid was then rinsed three times with three droplets of double distilled water each time removing the excess. Immediately after last rinse, the grid was exposed to the staining solution (1% uranyl formate) for 60 s and the stain was carefully removed using a fresh piece of filter paper. Finally, the grid was dried at room temperature for 2 h and used for TEM analysis.

### Results and Discussion

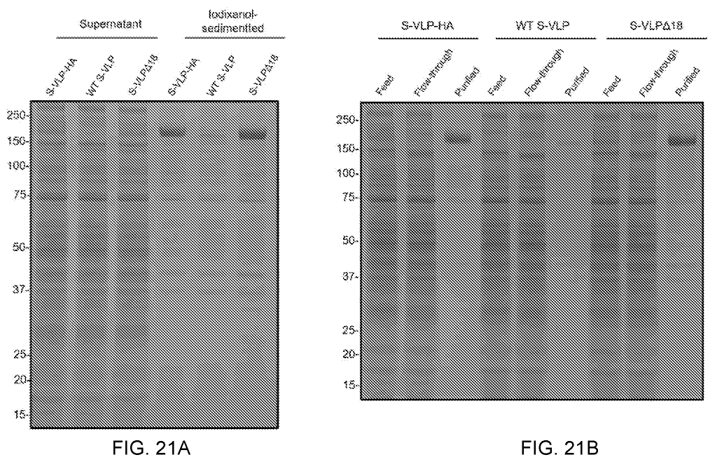

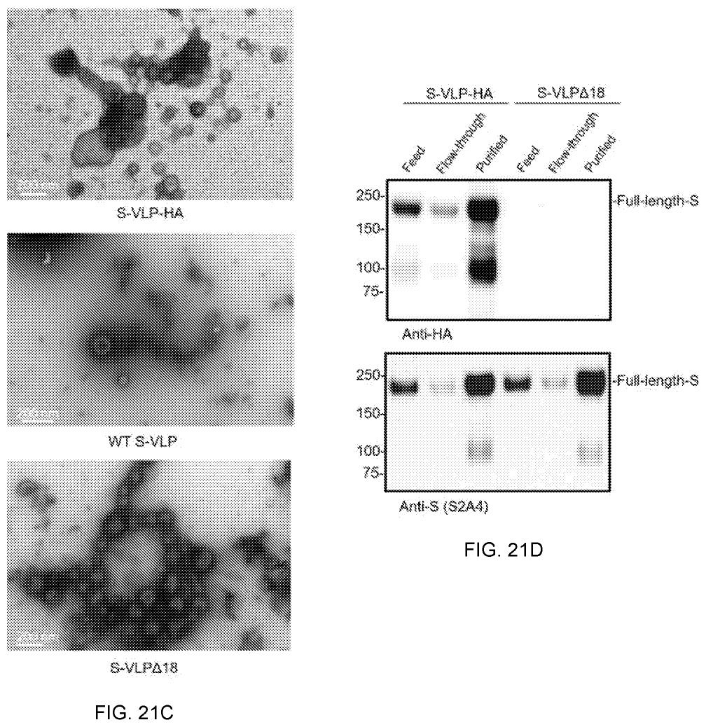

SARS-CoV-2 S is known to contain an endoplasmic reticulum retention signal at its C terminus. C-terminal truncation of S by 18-21 amino acids, which removes the ER retention signal, has been shown to enhance S incorporation in pseudotyped lentiviral and vesicular stomatitis virus (VSV) particles by several fold [Johnson et al, 2020; Fu et al, 2021; Schmidt et al, 2020]. To investigate whether production of high titers of S-VLPs depend on the presence or absence of the S C-tail, we tested the S expression vectors S-HA, wild type (WT) S (no HA) and S with an 18 amino acid deletion at the C-terminus (A18). Total protein staining of cushion-sedimented S-VLPs showed a high level of S for S-HA and SΔ18, but failed to detect S for the WT S condition (FIG. 21A). We next purified S-VLPs using affinity chromatography and observed low quantities of S-VLPs for the WT S condition. In contrast, similar high concentrations of S-VLP in S-HA and SΔ18 conditions were detected by total protein staining, western blot and TEM (FIGS. 21B-D).

Taken together, these results demonstrate that overexpression of the full-length S protein is sufficient to form S-VLPs, but modification of the S C-terminal tail leads to increased production. Without wishing to be bound by theory, the present inventors hypothesize that the presence of the HA tag at the C-terminus masks or interferes with the ER retention signal, allowing for efficient S-VLP production of the HA-tagged full-length S protein, similar to the truncated mutant.

### Example 4: Production of Bivariant S-VLPs

### Materials and Methods

### Cells and Culture Conditions

These methods were carried out as described in Example 1.

### Plasmid Constructs

The coding sequences for SARS-CoV-2 S and S-delta18 (SΔ18) were as described in Example 3. Expression constructs for variant spike proteins were prepared by re-synthesizing and replacing restriction fragments encompassing mutations present in omicron B.1.1.529 (A67V, H69del, V70del, T95I, G142D, V143del, Y144del, Y145del, N211del, L212I, E215del, E216del, E217del, F339D, S371L, S373P, S375F, K417N, N440K, G446S, S477N, T478K, E484A, Q493K, G496S, Q498R, N501Y, Y505H, T547K, D614G, H₆₅₅Y, N679K, P681H, N764K, D796Y, N856K, Q954H, N969K, L981F) and omicron BA4/BA5 (T19I, L24del, P25del, P26del, A27S, H₆₉del, V70del, G142D, V213G, G339D, S371F, S373P, S375F, T376A, D405N, R408S, K417N, N440K, L452R, S477N, T478K, E484A, F486V, Q498R, N501Y, Y505H, D614G, H₆₅₅Y, N679K, P681H, N764K, D796Y, Q954H, N969K) variants. Plasmids were sequenced, produced and purified as described in Example 3.

### Cell Transfections

CHO cell transfection methods were performed as described in Stuible et al, 2021. Co-transfection of structural SARS-CoV-2 genes for bivariant VLPs was performed using a mixture of plasmids comprising 59.5% pTT5-Spike (Δ18), 25.5% pTT5-Spike (B.1.1.529)-HA or 25.5% pTT5-Spike (BA4/BA5))-HA, 10% Bcl-XL and 5% GFP diluted in CHO complete media.

### SARS-CoV-2 VLP Harvesting and Sedimentation, SARS-CoV-2 VLP and S Purification by Affinity Chromatography, Total Protein Quantification, SDS-PAGE and Immunoblotting, and Negative-Stain Transmission Electron Microscopy (TEM)

These methods were carried out as described in Example 3.

### Results and Discussion

To evaluate the incorporation of different S variants into the same VLP, CHO-C2 cells were co-transfected with SΔ18 ancestral strain plasmid along with a 30% (from total S DNA) of tagged S-HA plasmid corresponding to Omicron B.1.1.529 or Omicron BA4/BA5 sequences. These plasmids were used to generate S-VLPs, as described in Example 1. The resulting VLPs were then purified using A VIPure-COV2S VLP affinity resin, which contains ligands that interact specifically with the ancestral strain (Wuhan) but not with Omicron-variant S proteins. Therefore, monovariant Omicron S-VLPs should be washed away during the purification process.

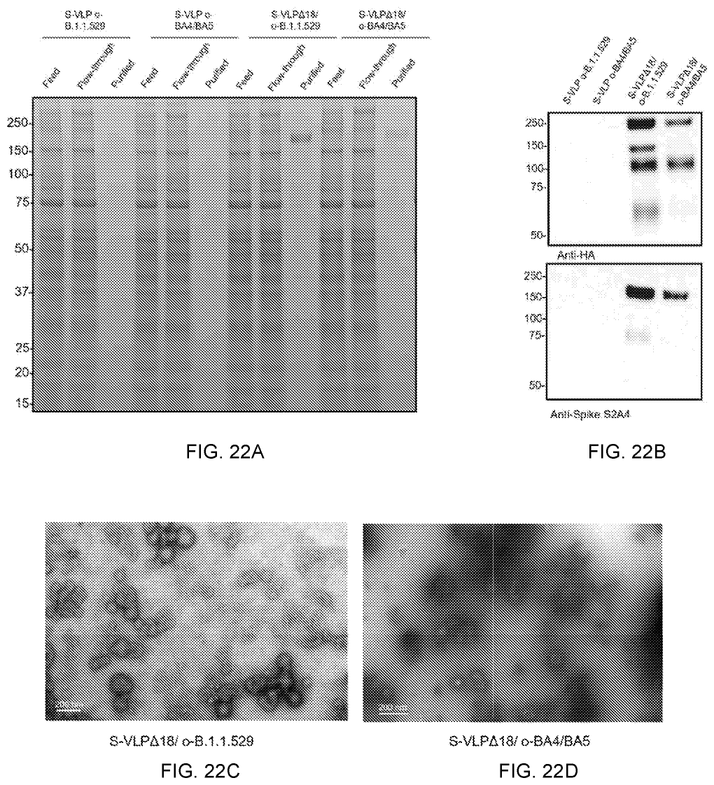

As shown in FIG. 22A, S-VLPs containing S ancestral antigens were effectively purified whereas VLPs harboring only Omicron S antigens were not, as expected. Next, we performed immunoblots on purified products with anti-HA to determine the presence of Omicron antigens. As shown in FIG. 22B, purified S-VLPs contain both ancestral and Omicron SARS-CoV-2 S antigens. Finally, TEM analysis showed effective formation and purification of bivariant S-VLPs (FIG. 22C). These results demonstrate that S-VLPs produced in CHO-C2 cells can incorporate S antigens of multiple SARS-CoV-2 variants.

### Example 5: Production of Bivalent S-VLPs

### Cells and Culture Conditions

These methods were carried out as described in Example 1.

### Plasmid Constructs

The coding sequences for SARS-CoV-2 S and S-delta18 S (SΔ18) were as described in Example 3. The coding sequences for the hemagglutinin (H1) and neuraminidase (N1) proteins from Influenza A virus (strain A/Puerto Rico/8/1934 H1N1) were obtained from the reference sequences EF467821.1 and NC_002018.1, respectively. The amino acid sequence for H1 is provided in SEQ ID NO: 37 and the amino acid sequence for N1 is provided in SEQ ID NO: 38. The coding sequence for the Respiratory syncytial virus (RSV) fusion (F) protein was obtained from the reference sequence EF566942.1. The amino acid sequence of RSV F is provided in SEQ ID NO: 39. Muscarinic acetylcholine receptor M4 (NP_000732.2) and Galanin receptor type 2 (GALR2 (NP_003848.1)) human sequences were codon optimized for CHO cells. The amino acid sequence of M4 is provided in SEQ ID NO: 40 and the amino acid sequence of GALR2 is provided in SEQ ID NO: 41. Both sequences were flanked at the N-terminus with a Flag tag (DYKDDDDK, SEQ ID NO: 42) and at the C-terminal domain with an HA tag (YPYDVPDYA, SEQ ID NO: 43). All gene sequences were synthesized at GenScript, Piscataway, NJ, USA and sub-cloned in pTT5® plasmid using EcoRI, BamHI, or KpnI (New England Biolabs). Plasmids were produced and purified as described in Example 3.

### Cell Transfections

CHO cell transfection methods were performed as described in Stuible et al, 2021. Co-transfection for bivalent S—H1, S—H1/N1, S—RSVF VLPs was carried out in CHO media at 37.5% pTT5-Spike-HA, 37.5% pTT5-RSVF or pTT5-H1, 20% Bcl-XL and 5% GFP. Generation of multivalent S-H1-N1 was performed by co-transfection of pTT5-Spike-HA, pTT5-H1 and pTT5-N1 with 20% Bcl-XL and 5% GFP.

### SARS-CoV-2 VLP Harvesting and Sedimentation, SARS-CoV-2 VLP and S Purification by Affinity Chromatography, Total Protein Quantification, SDS-PAGE and Immunoblotting, and Negative-Stain Transmission Electron Microscopy (TEM)

These methods were carried out as described in Example 3.

### Immunogold Labeling and TEM for Bivalent S/H₁-VLPs

TEM grids were placed carbon-side down onto 15 μL of purified S/H1-VLP sample and incubated for 1 min (all incubations are performed at room temperature (RT) in a humidified chamber), followed by 1 min further incubation with distilled water. Grids were then transferred to 15 μL blocking buffer (99 mL PBS+1 mL Probumin-BSA 30%) and incubated for 20 min. Subsequently, the excess droplets in grids were wicked away and grids were transferred carbon-side down to 15 μL primary antibody cocktail or negative control antibody cocktail for 2 h. Primary antibodies consist of NRC: VHH-72-hFc anti-Spike, mouse anti-HA (H1) (Santa Cruz IV.C102, sc-80550). Anti-RSVF (F148-4B7-1), and mouse anti-FLAG (Sigma #F3165) at final concentrations of 1 μg/mL. The negative control antibody cocktail consists of mouse anti-GST (Santa Cruz sc-138), in-house human Anti-F(RSV) (only for bivalent Influenza conditions) and Human anti-IgG3K (Sigma 15654) at a final concentration of 1 μg/mL. Next, grids were washed with 15 μl washing buffer solution (100 mL PBS+100 μL Probumin-BSA 30%) followed by 2 min incubation three times. Grids were then placed carbon-side down onto 15 μL gold-conjugated secondary antibody cocktail and incubated for 1 h. Gold-conjugated secondary antibodies comprised goat anti-human-gold 18 nm (Jackson ImmunoResearch, 109-215-088), and goat anti-mouse-gold 10 nm (Sigma Aldrich, G7652) at 1:20 dilution. Then, three washes with washing buffer solution and 2 min incubations were developed. Grids were incubated onto 15 μL of negative Nano Van staining solution (Nanoprobe) for 1 min. Finally, grids were left drying overnight at room temperature before TEM analysis as previously described.

### Results and Discussion

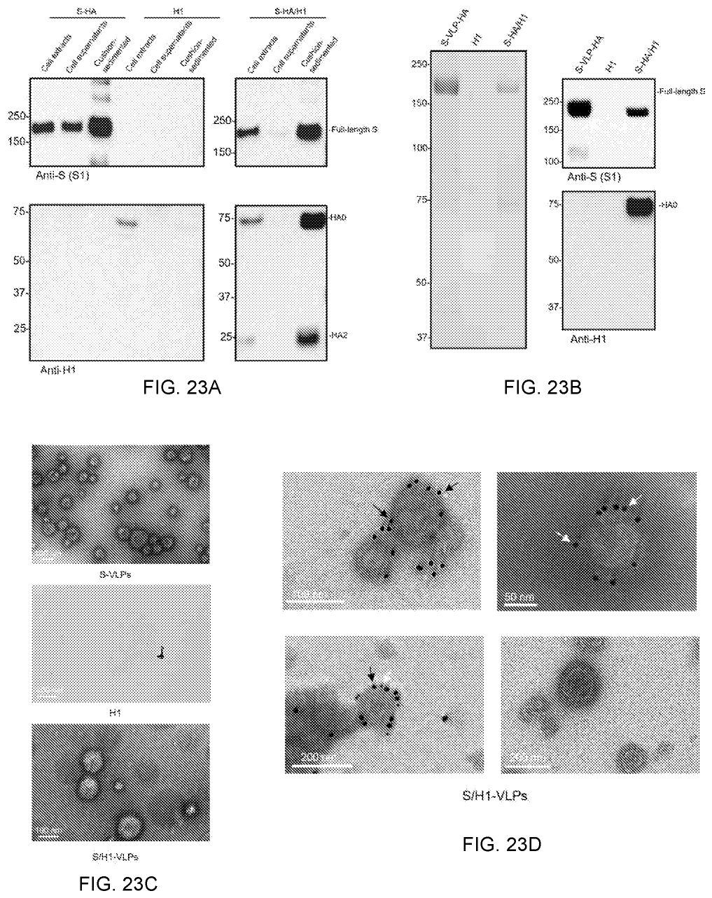

Based on the results described in Example 4, the present inventors hypothesized that S-VLPs could potentially integrate and display other antigens from different virus families. To evaluate this idea, CHO-C2 cells were transfected with vectors encoding S-HA, influenza A virus H1 (strain A/Puerto Rico/8/1934 H1N1), or both S-HA and influenza A virus H1. Cell extracts, supernatants and sedimented-cushion fractions were then analyzed. As shown in FIG. 23A, overexpression of H1 protein in CHO-C2 cells does not drive formation of VLPs, contrary to S-HA, as detected by western blotting. Strikingly, cells co-transfected with S-HA/H1 plasmids release large amounts of uncleaved and cleaved H1 (HA0 and HA2) protein observable in cushion-sedimented fractions, suggesting the presence of S-VLPs harboring influenza H1 antigens (FIG. 23A). Affinity chromatography was then used for purification to specifically capture S and examine whether S-VLPs in co-expressing conditions contain H1 protein. Total protein staining, immunoblotting and TEM experiments demonstrated VLP formation as well as the presence of S and H1 antigens (FIGS. 23B and C). To investigate the presence of S and H1 proteins in the same VLPs (bivalent VLPs), both antigens were visualized by immunolabeling with gold particles for S (gold 18 nm) and H1 (gold 10 nm). FIG. 23D depicts individual or double gold labeling of S and H1 proteins corroborating the presence of both antigens at the surface of the same VLP particles.

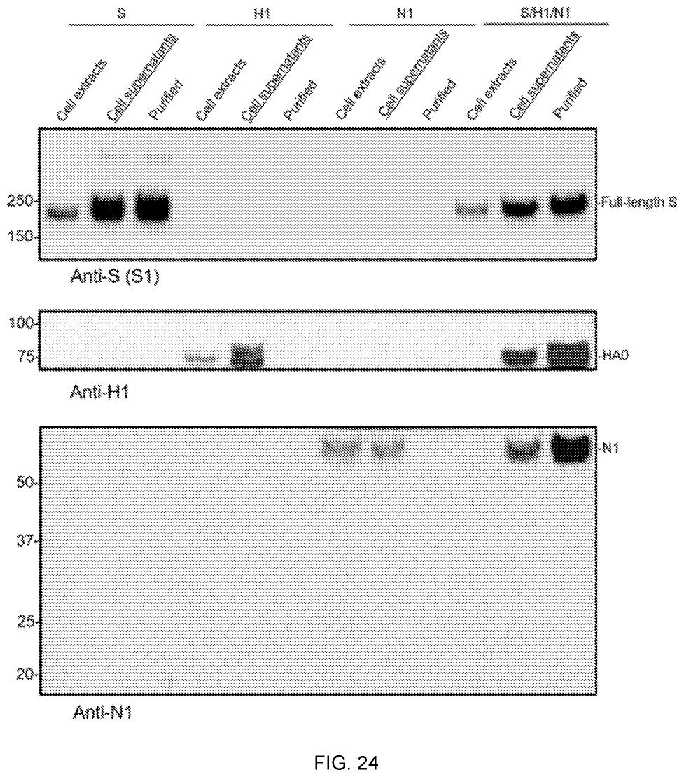

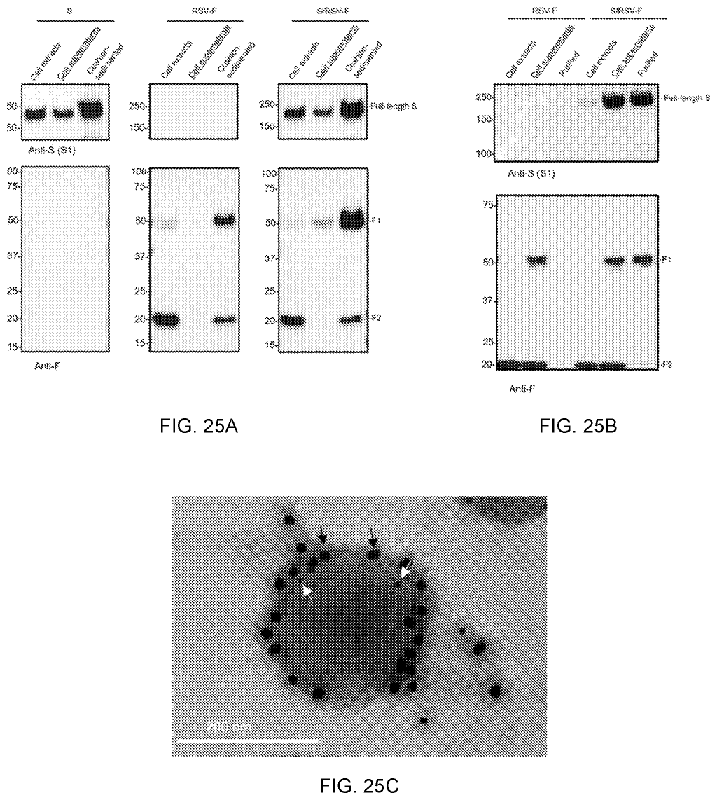

The potential of S-VLPs to incorporate multiple virus antigens was explored by expressing three different genes simultaneously: influenza A H1 and N1 genes were co-expressed with S-HA in CHO-C2 cells. Western blotting analysis of the purified samples indicates that all three viral proteins can be found in the VLPs induced by S-HA expression (FIG. 24). The F protein of RSV, another respiratory virus, can also be found in sedimented and purified S-VLPs upon co-expression with S constructs (FIGS. 25A-C). Together, these results indicate that CHO-derived S-VLPs provide a flexible platform to display a variety of viral surface antigens in addition to different SARS-CoV-2 variant S proteins.

### Example 6: Production of S-VLPs Comprising Non-Viral Cell-Surface Proteins

### Materials and Methods

### Cells and Culture Conditions

These methods were carried out as described in Example 1.

### Plasmid Constructs

The coding sequences for SARS-CoV-2 S and Sm-delta18 S (SΔ18) were as described in Example 3. The coding sequences for M4 and GALR2 were as described in Example 5. All gene sequences were synthesized at GenScript, Piscataway, NJ, USA and sub-cloned in pTT5® plasmid using EcoRI BamHI or KpnI (New England Biolabs). Plasmids were produced and purified as described in Example 3.

### Cell Transfections

CHO cell transfection methods were performed as described in Alpuche-Lazcano et al., 2023 and in Stuible et al, 2021. Co-transfection for bivalent Sdelta18/M4 and Sdelta18/GALR2 VLPs was carried out in CHO media at 37.5% pTT5-Spike-delta18, 37.5% pTT5-M4 or pTT5-GALR2, 20% Bcl-X_{L} and 5% GFP.

### SARS-CoV-2 VLP Harvesting and Sedimentation, SARS-CoV-2 VLP and S Purification by Affinity Chromatography, and SDS-PAGE and Immunoblotting

These methods were carried out as described in Example 3.

### Results and Discussion

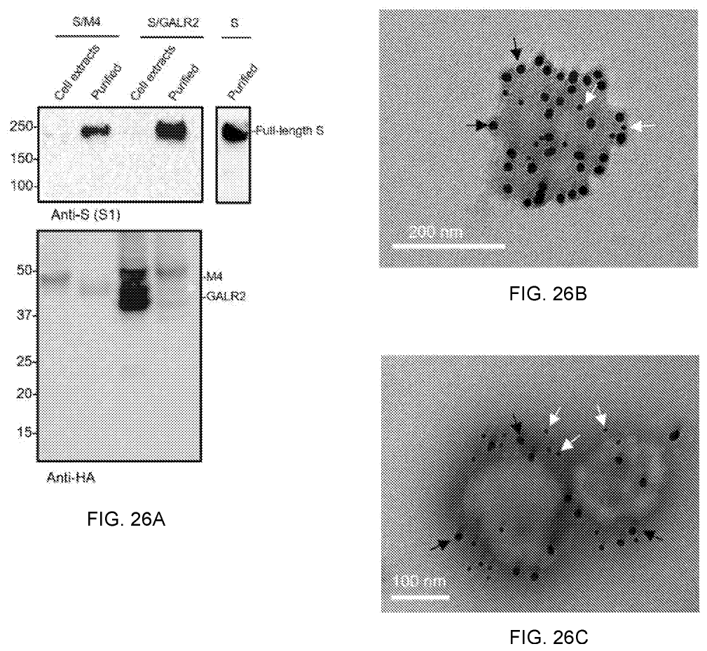

The present inventors evaluated whether two human cell-surface receptors, the M4 and GALR2 GPCRs, can be displayed on S-VLPs. Remarkably, 5 days post-co-transfection of these GPCRs with SΔ18, immunoblot experiments on affinity-purified samples indicated an effective incorporation of M4 and GALR2 into S-VLPs (FIGS. 26A-C). This indicates that the function of S-VLPs is not limited to virus vaccine antigen carriers. A variety of applications for this technology could be envisioned, but for M4 and GALR2, these VLPs could be particularly useful for therapeutic antibody development applications, as these receptors are particularly difficult to produce as soluble recombinant proteins.

### Example 7: Protein Content Analysis of eVLPs

### Materials and Methods

Total protein from purified monovariant (FL-S-HA), bivariant (SΔ18/S-omicron-B.1.1.529-HA), bivalent (FL-S-HA/H1), and trivalent (FL-S-HA/H1/N1) eVLPs was run on SDS-PAGE gels as described in Example 3, and stained with Coomassie blue or SYPRO® Ruby. The gels were then subject to densitometric analysis. Image Lab software (2020 Bio-Rad Laboratories. Inc) was used to calculate the band percentage of protein of interest relative to the percent of total protein detected by densitometry in SDS-PAGE gels. The background subtraction cut-off was relative to the intensity of each gel staining.

### Results and Discussion

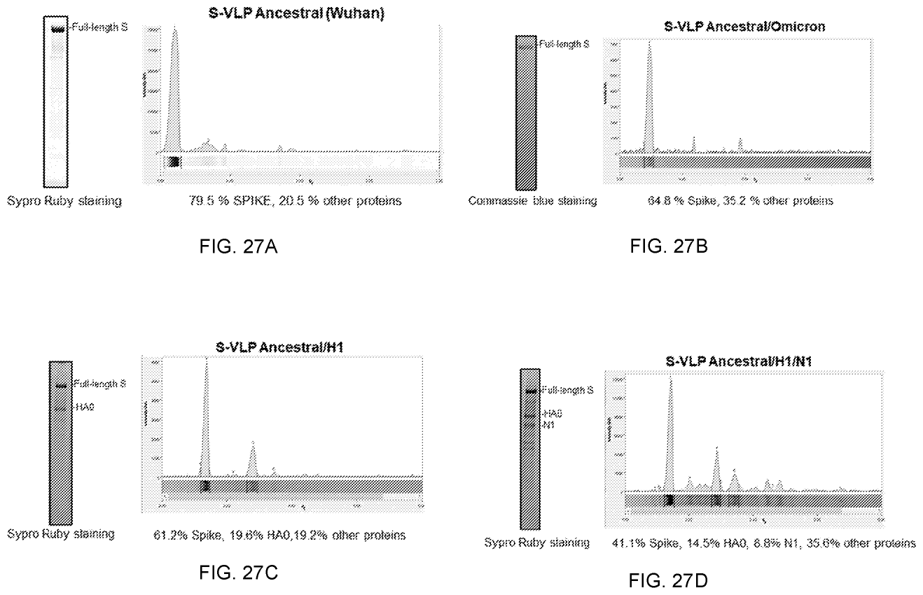

Representative SDS-PAGE gels and densitometric plots are shown in FIGS. 27A to 27D. As shown in FIG. 27A, the monovariant eVLP (FL-S-HA, Wuhan) was found to contain 79.5% of FL-S-HA protein relative to total protein. As shown in FIG. 27B, the bivariant eVLP (SΔ18/FL-S-omicron-B.1.1.529-HA) was found to contain 64.8% of S protein (SΔ18 and FL-S-omicron-B.1.1.529-HA combined) relative to total protein. As shown in FIG. 27C, the bivalent (FL-S-HA/H1) eVLP was found to contain 61.2% of FL-S-HA protein and 19.6% of H1 protein, relative to total protein. As shown in FIG. 27D, the trivalent (FL-S-HA/H1/N1) eVLP was found to contain 41.1% of FL-S-HA protein, 14.5% of H1 protein and 8.8% of N1 protein, relative to total protein.

The preceding examples have been provided to illustrate various aspects of the disclosure and are non-limiting. The scope of the claims is not limited to the specific details provided in the examples; rather the claims are to be given the broadest interpretation consistent with the teachings of the disclosure as a whole, with consideration to the common general knowledge of a person skilled in the art to which the present disclosure pertains.

### REFERENCES

The contents of each of the following references is herein incorporated by reference in its entirety.
Akache, B. et al. Immunogenic and efficacious SARS-CoV-2 vaccine based on resistin-trimerized spike antigen SmT1 and SLA archaeosome adjuvant. Scientific reports 11, 21849 (2021).
D'Aoust, M. A. et al. Influenza virus-like particles produced by transient expression in Nicotiana benthamiana induce a protective immune response against a lethal viral challenge in mice. Plant biotechnology journal 6, 930-940 (2008).
de Wit, E. et al. The Middle East respiratory syndrome coronavirus (MERS-COV) does not replicate in Syrian hamsters. PloS one 8, e69127 (2013).
Dobro, M. J., Melanson, L. A., Jensen, G. J. & McDowall, A. W. Plunge freezing for electron cryomicroscopy. Methods in enzymology 481, 63-82 (2010).
Duan Liangwei, Zheng Qianqian, Zhang Hongxia, Niu Yuna, Lou Yunwei, Wang Hui, The SARS-CoV-2 Spike Glycoprotein Biosynthesis, Structure, Function, and Antigenicity: Implications for the Design of Spike-Based Vaccine Immunogens, Frontiers in Immunology. 11:576622 (2020).
Duroy, P. O. et al. Characterization and mutagenesis of Chinese hamster ovary cells endogenous retroviruses to inactivate viral particle release. Biotechnology and bioengineering 117, 466-485 (2020).
Fluckiger, A. C. et al. An enveloped virus-like particle vaccine expressing a stabilized prefusion form of the SARS-CoV-2 spike protein elicits highly potent immunity. Vaccine 39, 4988-5001 (2021).
Fu, X., L. Tao, and X. Zhang, Comprehensive and systemic optimization for improving the yield of SARS-CoV-2 spike pseudotyped virus. Molecular Therapy-Methods & Clinical Development, 2021. 20: p. 350-356.
Fuenmayor, J., Gòdia, F. & Cervera, L. Production of virus-like particles for vaccines. New biotechnology 39, 174-180 (2017).
Hsieh, C. L. et al. Structure-based design of prefusion-stabilized SARS-CoV-2 spikes. Science. 369(6510), 1501-1505 (2020).
Isho, B. et al. Persistence of serum and saliva antibody responses to SARS-CoV-2 spike antigens in COVID-19 patients. Science immunology 5 (2020).
Johnson, M. C., et al., Optimized Pseudotyping Conditions for the SARS-CoV-2 Spike Glycoprotein. Journal of Virology, 2020. 94 (21). Joubert S., et al., A CHO stable pool production platform for rapid clinical development of trimeric SARS-CoV-2 spike subunit vaccine antigens. Biotechnology and Bioengineering. 2023 Mar. 29.
Juraszek, J., Rutten, L., Blokland, S. et al. Stabilizing the closed SARS-CoV-2 spike trimer. Nat Commun 12, 244 (2021).
Lieber, M. M., Benveniste, R. E., Livingston, D. M. & Todaro, G. J. Mammalian cells in culture frequently release type C viruses. Science 182, 56-59 (1973).
Martínez-Flores, D. et al. SARS-CoV-2 Vaccines Based on the Spike Glycoprotein and Implications of New Viral Variants. Frontiers in immunology 12, 701501 (2021).
Pecetta, S., Kratochvil, S., Kato, Y., Vadivelu, K. & Rappuoli, R. Immunology and Technology of Severe Acute Respiratory Syndrome Coronavirus 2 (SARS-CoV-2) Vaccines. Pharmacological reviews 74, 313-339 (2022).
Plotkin, S. History of vaccination. Proceedings of the National Academy of Sciences of the United States of America 111, 12283-12287 (2014).
Poulain, A. et al. Rapid protein production from stable CHO cell pools using plasmid vector and the cumate gene-switch. Journal of biotechnology 255, 16-27 (2017).
Riley T P, Chou H T, Hu R, et al. Enhancing the Prefusion Conformational Stability of SARS-CoV-2 Spike Protein Through Structure-Guided Design. Front Immunol. 12:660198 (2021).
Schaub, J. M., Chou, C W., Kuo, H C. et al. Expression and characterization of SARS-CoV-2 spike proteins. Nat Protoc 16, 5339-5356 (2021).
Schmidt, F., et al., Measuring SARS-CoV-2 neutralizing antibody activity using pseudotyped and chimeric viruses. Journal of Experimental Medicine, 2020. 217(11).
Stocks, B. B., Thibeault, M. P., Schrag, J. D. & Melanson, J. E. Characterization of a SARS-CoV-2 spike protein reference material. Analytical and bioanalytical chemistry, 1-9 (2022).
Stuible, M. et al. Rapid, high-yield production of full-length SARS-CoV-2 spike ectodomain by transient gene expression in CHO cells. Journal of biotechnology 326, 21-27 (2021).
Sun, J. & Li, H. How to operate a cryo-electron microscope. Methods in enzymology 481, 231-249 (2010).
Swann, H. et al. Minimal system for assembly of SARS-CoV-2 virus like particles. Scientific reports 10, 21877 (2020).
Ward, B. J. et al. Phase 1 randomized trial of a plant-derived virus-like particle vaccine for COVID-19. Nature medicine 27, 1071-1078 (2021).
Wrapp, D. et al. Cryo-EM structure of the 2019-nCOV spike in the prefusion conformation. Science 367, 1260-1263 (2020a).
Wrapp, D. et al. Structural Basis for Potent Neutralization of Betacoronaviruses by Single-Domain Camelid Antibodies. Cell 181, 1004-1015.e1015 (2020b).
Xia X. Domains and Functions of Spike Protein in Sars-Cov-2 in the Context of Vaccine Design. Viruses. 13 (1), 109 (2021).
Xu, R., Shi, M., Li, J., Song, P. & Li, N. Construction of SARS-CoV-2 Virus-Like Particles by Mammalian Expression System. Frontiers in bioengineering and biotechnology 8, 862 (2020).
Yilmaz, I. C. et al. Development and preclinical evaluation of virus-like particle vaccine against COVID-19 infection. Allergy 77, 258-270 (2022).

What is claimed is:

1. An enveloped virus-like particle (eVLP) derived from an animal host cell, wherein the eVLP comprises a substantially full-length recombinant SARS-CoV-2 spike protein.

2. The eVLP of claim 1, wherein the substantially full-length recombinant SARS-CoV-2 spike protein comprises a mutation that reduces or eliminates the function of the endoplasmic reticulum (ER) retention signal of the spike protein.

3. The eVLP of claim 2, wherein the mutation that reduces or eliminates the function of the ER retention signal is a C-terminal truncation of 5 to 21 contiguous amino acids relative to the full-length SARS-CoV-2 spike protein sequence.

4. The eVLP of any one of claims 1 to 3, wherein the substantially full-length recombinant SARS-CoV-2 spike protein is a chimeric protein further comprising a heterologous polypeptide fused to the C-terminus of the SARS-CoV-2 spike protein sequence.

5. The eVLP of any one of claims 1 to 4, wherein the substantially full-length recombinant SARS-CoV-2 spike protein is stabilized in the prefusion conformation.

6. The eVLP of any one of claims 1 to 5, wherein the substantially full-length recombinant SARS-CoV-2 spike protein comprises an inactivated S1/S2 furin cleavage site.

7. The eVLP of any one of claims 1 to 4, wherein the substantially full-length recombinant SARS-CoV-2 spike protein comprises an amino acid sequence having at least 70% sequence identity to the full length of the amino acid sequence set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47.

8. The eVLP of any one of claims 1 to 4, wherein the substantially full-length recombinant SARS-CoV-2 spike protein comprises an amino acid sequence as set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47, except that the amino acid sequence comprises between one and 60 amino acid substitutions, insertions, and/or deletions relative to the amino acid sequence set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47.

9. The eVLP of any one of claims 1 to 4, wherein the substantially full-length recombinant SARS-CoV-2 spike protein comprises an amino acid sequence as set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47, except that the amino acid sequence comprises between one and 20 conservative amino acid substitutions relative to the amino acid sequence set forth in SEQ ID NO: 2, SEQ ID NO: 4, SEQ ID NO: 5, SEQ ID NO: 7, SEQ ID NO: 8, SEQ ID NO: 10, SEQ ID NO: 11, SEQ ID NO: 13, SEQ ID NO: 14, SEQ ID NO: 16, SEQ ID NO: 18, SEQ ID NO: 20, SEQ ID NO: 22, SEQ ID NO: 24, SEQ ID NO: 44, SEQ ID NO: 45, SEQ ID NO: 46, or SEQ ID NO: 47.

10. The eVLP of any one of claims 1 to 9, wherein the eVLP comprises one or more membrane components from the host cell.

11. The eVLP of any one of claims 1 to 10, wherein the host cell is a mammalian cell.

12. The eVLP of claim 11, wherein the mammalian cell is a CHO cell.

13. The eVLP of any one of claims 1 to 12, wherein the eVLP does not comprise one or more of: SARS-CoV-2 envelope protein, SARS-CoV-2 membrane protein, and SARS-CoV-2 nucleocapsid protein.

14. The eVLP of any one of claims 1 to 13, wherein the substantially full-length recombinant SARS-CoV-2 spike protein makes up at least 50% of the total protein content of the eVLP.

15. The eVLP of any one of claims 1 to 13, wherein the eVLP is a multivalent eVLP further comprising an additional recombinant protein.

16. The eVLP of claim 15, wherein the additional recombinant protein comprises a non-viral recombinant protein.

17. The eVLP of claim 16, wherein the additional recombinant protein comprises a cell surface protein.

18. The eVLP of claim 15, wherein the additional recombinant protein comprises a viral antigen from a virus other than SARS-CoV-2.

19. The eVLP of any one of claims 15 to 19, wherein the additional recombinant protein comprises an influenza A antigen.

20. The eVLP of any one of claims 15 to 19, wherein the substantially full-length recombinant SARS-CoV-2 spike protein makes up at least 25% of the total protein content of the eVLP.

21. The eVLP of claim 19 or 20, wherein the additional recombinant protein comprises influenza hemagglutinin or neuraminidase.

22. The eVLP of any one of claims 1 to 20 wherein the eVLP does not comprise any recombinant protein, other than the substantially full-length recombinant SARS-CoV-2 spike protein, that is substantially capable of independently inducing eVLP formation by the host cell.

23. The eVLP of any one of claims 1 to 13, wherein the eVLP is a multivariant eVLP comprising at least one additional substantially full-length recombinant SARS-CoV-2 spike protein having a different amino acid sequence from the substantially full-length recombinant SARS-CoV-2 spike protein.

24. The eVLP of claim 23, wherein the eVLP does not comprise any recombinant protein, other than the substantially full-length recombinant SARS-CoV-2 spike protein and the at least one additional substantially full-length recombinant SARS-CoV-2 spike protein, that is substantially capable of independently inducing eVLP formation by the host cell.

25. The eVLP of claim 23 or 24, wherein the substantially full-length recombinant SARS-CoV-2 spike protein and the at least one additional substantially full-length recombinant SARS-CoV-2 spike protein together make up at least 50% of the total protein content of the eVLP.

26. The eVLP of claim 23 or 24, wherein the eVLP further comprises an additional recombinant protein, wherein the additional recombinant protein is as defined in any one of claims 15 to 21.

27. The eVLP of any one of claims 1 to 26, wherein the eVLP is isolated or purified.

28. The eVLP of any one of claims 1 to 27, wherein the eVLP has a diameter of about 50 nm to about 150 nm.

29. A composition comprising a plurality of eVLPs as defined in any one of claims 1 to 27, wherein the eVLPs in the composition have a median diameter of about 115 to about 135 nm.

30. A method for preparing an enveloped virus-like particle (eVLP), the method comprising:
(a) providing an animal host cell comprising within its nucleus a nucleic acid molecule encoding a substantially full-length recombinant SARS-CoV-2 spike protein;
(b) incubating the host cell in a medium under conditions that allow the substantially full-length recombinant SARS-CoV-2 spike protein to be expressed by the host cell; and
(c) allowing the host cell to produce the eVLP and release the eVLP into the medium,
wherein the substantially full-length recombinant SARS-CoV-2 spike protein is as defined in any one of claims 1 to 9.

31. The method of claim 30, wherein the host cell is a mammalian host cell.

32. The method of any claim 31, wherein the mammalian cell is a CHO cell.

33. The method of claim 32, wherein the CHO cell comprises a mutation that inactivates a functional endogenous retrovirus sequence in its genome.

34. The method of any one of claims 30 to 33, further comprising isolating the eVLP from the medium.

35. The method of any one of claims 30 to 34, wherein the method results in the production of at least 1.0×10¹² eVLPs per liter of culture.

36. The method of any one of claims 30 to 35, wherein the method results in the production of a plurality of eVLPs having a median diameter of about 115 to about 135 nm.

37. The method of any one of claims 30 to 36, wherein step (a) comprises transfecting the host cell with the nucleic acid molecule.

38. The method of any one of claims 30 to 37, wherein the nucleic acid molecule is a plasmid.

39. The method of any one of claims 30 to 38, wherein the nucleic acid molecule comprises a nucleotide sequence encoding the substantially full-length recombinant SARS-CoV-2 spike protein operatively linked to a heterologous regulatory element that is operative in the host cell to allow the substantially full-length recombinant SARS-CoV-2 spike protein to be expressed by the host cell.

40. The method of claim 39, wherein the nucleic acid molecule is codon-optimized for expression in the host cell.

41. The method of claim 40, wherein the nucleic acid molecule comprises the nucleotide sequence set forth in SEQ ID NO: 3, SEQ ID NO: 6, SEQ ID NO: 9, SEQ ID NO: 12, SEQ ID NO: 15, SEQ ID NO: 17, SEQ ID NO: 19, SEQ ID NO: 21, SEQ ID NO: 23, or SEQ ID NO: 49.

42. The method of any one of claims 30 to 41, wherein the host cell comprises within its nucleus a nucleic acid molecule encoding at least one additional substantially full-length recombinant SARS-CoV-2 spike protein having a different amino acid sequence than the substantially full-length recombinant SARS-CoV-2 spike protein.

43. The method of any one of claims 30 to 41, wherein the substantially full-length SARS CoV-2 spike protein makes up at least 50% of the total protein content of the eVLP produced by the method.

44. The method of claim 42, wherein the substantially full-length recombinant SARS-CoV-2 spike protein and the at least one additional substantially full-length recombinant SARS-CoV-2 spike protein together make up at least 50% of the total protein content of the eVLP produced by the method.

45. The method of any one of claims 30 to 42, wherein the host cell provided in step (a) further comprises within its nucleus a nucleic acid molecule encoding an additional recombinant protein operatively linked to a regulatory element that is operative in the cell to allow the additional recombinant protein to be expressed by the cell.

46. The method of claim 45, wherein the additional recombinant protein comprises a non-viral protein.

47. The method of claim 46, wherein the additional recombinant protein comprises a cell surface protein.

48. The method of claim 47, wherein the additional recombinant protein comprises a viral antigen other than SARS-CoV-2 S.

49. The method of claim 48, wherein the viral antigen is an influenza A antigen.

50. The method of any one of claims 45 to 49, wherein the substantially full-length recombinant SARS-CoV-2 spike protein makes up at least 25% of the total protein content of the eVLP produced by the method.

51. The method of any one of claims 48 to 50, wherein the additional recombinant protein comprises influenza hemagglutinin or neuraminidase.

52. The method of any one of claims 30 to 50, wherein the host cell does not co-express together with the substantially full-length recombinant SARS-CoV-2 spike protein any viral protein, other than the substantially full-length recombinant SARS-CoV-2 spike protein, that is substantially capable of independently inducing eVLP formation by the host cell.

53. The method of any one of claims 30 to 52, wherein the host cell does not co-express together with the substantially full-length recombinant SARS-CoV-2 spike protein one or more of: SARS-CoV-2 envelope protein, SARS-CoV-2 membrane protein, and SARS-CoV-2 nucleocapsid protein.

54. An animal cell for the production of an enveloped virus-like particle (eVLP) comprising a substantially full-length recombinant SARS-CoV-2 spike protein, wherein the cell comprises within its nucleus a nucleic acid molecule encoding the substantially full-length recombinant SARS-CoV-2 spike protein operatively linked to a heterologous regulatory element that is operative in the cell to allow the substantially full-length recombinant SARS-CoV-2 spike protein to be expressed by the cell, wherein the substantially full-length recombinant SARS-CoV-2 spike protein is as defined in any one of claims 1 to 9.

55. The cell of claim 54, wherein the cell is a mammalian cell.

56. The cell of claim 55, wherein the mammalian cell is a CHO cell.

57. The cell of claim 56, wherein the CHO cell comprises a mutation that inactivates a functional endogenous retrovirus sequence in its genome.

58. The cell of any one of claims 54 to 57, wherein the cell further comprises within its nucleus a nucleic acid molecule encoding an additional recombinant protein operatively linked to a regulatory element that is operative in the cell to allow the additional recombinant protein to be expressed by the cell.

59. The cell of claim 58, wherein the additional recombinant protein comprises a non-viral protein.

60. The cell of claim 58, wherein the additional recombinant protein comprises a cell surface protein.

61. The cell of claim 58, wherein the additional recombinant protein comprises a viral antigen other than SARS-CoV-2 S.

62. The cell of claim 61, wherein the additional recombinant protein comprises an influenza A antigen.

63. The cell of claim 61, wherein the additional recombinant protein comprises influenza hemagglutinin or neuraminidase.

64. The cell of any one of claims 54 to 62, wherein the cell does not comprise any viral protein, other than the substantially full-length recombinant SARS-CoV-2 spike protein, that is capable of independently inducing eVLP formation by the host cell.

65. The cell of any one of claims 54 to 64, wherein the cell does not comprise one or more of: SARS-CoV-2 envelope protein, SARS-CoV-2 membrane protein, and SARS-CoV-2 nucleocapsid protein.

66. The cell of any one of claims 54 to 65, wherein the nucleic acid molecule is codon-optimized for expression in the cell.

67. The cell of claim 66, wherein the nucleic acid molecule comprises the nucleotide sequence set forth in SEQ ID NO: 3, SEQ ID NO: 6, SEQ ID NO: 9, SEQ ID NO: 12, SEQ ID NO: 15, SEQ ID NO: 17, SEQ ID NO: 19, SEQ ID NO: 21, SEQ ID NO: 23, or SEQ ID NO: 49.

68. The cell of any one of claims 54 to 67, wherein the cell comprises within its nucleus a nucleic acid molecule encoding a second substantially full-length recombinant SARS-CoV-2 spike protein having a different amino acid sequence from the substantially full-length recombinant SARS-CoV-2 spike protein, operatively linked to a heterologous regulatory element that is operative in the cell to allow the second substantially full-length recombinant SARS-CoV-2 spike protein to be expressed by the cell.

69. Use of the cell of any one of claims 54 to 68 to produce the eVLP comprising the substantially full-length recombinant SARS-CoV-2 spike protein.

70. A pharmaceutical composition comprising the eVLP of any one of claims 1 to 28, the composition of claim 29, an eVLP produced by the method of any one of claims 30 to 53, or an eVLP produced by the cell of any one of claims 54 to 68, and a pharmaceutically acceptable carrier or diluent.

71. The pharmaceutical composition of claim 70, further comprising an adjuvant.

72. The pharmaceutical composition of claim 71, wherein the adjuvant comprises 3-O-desacyl-4′-monophosphoryl lipid A (MPL) and/or a saponin.

73. The pharmaceutical composition of claim 72, wherein the adjuvant comprises MPL and QS-21.

74. The pharmaceutical composition of any one of claims 70 to 73, wherein the composition is an immunogenic composition or a vaccine composition.

75. A method of inducing an immune response in a subject, the method comprising administering to the subject the eVLP of any one of claims 1 to 28, the composition of claim 29, an eVLP produced by the method of any one of claims 30 to 53, an eVLP produced by the cell of any one of claims 54 to 68, or the composition of any one of claims 70 to 74.

76. A method for preventing COVID-19 or SARS-CoV-2 infection in a subject, the method comprising administering to the subject the eVLP of any one of claims 1 to 28, the composition of claim 29, an eVLP produced by the method of any one of claims 30 to 53, or an eVLP produced by the cell of any one of claims 54 to 68, or the composition of any one of claims 70 to 74.

77. The eVLP of any one of claims 1 to 28, the composition of claim 29, an eVLP produced by the method of any one of claims 30 to 53, an eVLP produced by the cell of any one of claims 54 to 68, or the composition of any one of claims 70 to 74, for use to induce an immune response in a subject.

78. The eVLP of any one of claims 1 to 28, the composition of claim 29, an eVLP produced by the method of any one of claims 30 to 53, an eVLP produced by the cell of any one of claims 54 to 68, or the composition of any one of claims 70 to 74, for use to prevent COVID-19 or SARS-CoV-2 infection.

79. Use of the eVLP of any one of claims 1 to 28, the composition of claim 29, an eVLP produced by the method of any one of claims 30 to 53, an eVLP produced by the cell of any one of claims 54 to 68, or the composition of any one of claims 70 to 74 to induce an immune response in a subject.

80. Use of the eVLP of any one of claims 1 to 28, the composition of claim 29, an eVLP produced by the method of any one of claims 30 to 53, an eVLP produced by the cell of any one of claims 54 to 68, or the composition of any one of claims 70 to 74 to prevent COVID-19 or SARS-CoV-2 infection.

81. Use of the eVLP of any one of claims 1 to 28, the composition of claim 29, an eVLP produced by the method of any one of claims 30 to 53, an eVLP produced by the cell of any one of claims 54 to 68, or the composition of any one of claims 70 to 74 in the preparation of a medicament for the prevention of COVID-19 or SARS-CoV-2 infection.
